(19) United States
(12) Patent Application Publication
(10) Pub. No.: US 2013/0266557 A1
(43) Pub. Date: Oct. 10, 2013

(54) METHODS FOR TREATMENT OF A SARCOMA USING AN EPIMETABOLIC SHIFTER (COENZYME Q10)

(71) Applicants: Rangaprasad Sarangarajan, Boylston, MA (US); Niven Rajin Narain, Cambridge, MA (US); John Patrick McCook, Frisco, TX (US)

(72) Inventors: Rangaprasad Sarangarajan, Boylston, MA (US); Niven Rajin Narain, Cambridge, MA (US); John Patrick McCook, Frisco, TX (US)

(21) Appl. No.: 13/749,337

(22) Filed: Jan. 24, 2013

Related U.S. Application Data
(63) Continuation of application No. 12/868,678, filed on Aug. 25, 2010, now abandoned.
(60) Provisional application No. 61/236,845, filed on Aug. 25, 2009.

(51) Int. Cl. A61K 31/122; A61K 35/12; A61K 39/395

(52) U.S. Cl. CPC A61K 31/122; A61K 39/395; A61K 35/12
USPC 424/130.1; 424/94.1; 514/690; 424/93.7; 506/7; 506/9; 435/7.1

(57) ABSTRACT

Methods and formulations for treating a sarcoma in humans using an epimetabolic shifter, such as Coenzyme Q10, a building block of CoQ10, a derivative of CoQ10, an analog of CoQ10, a metabolite of CoQ10, or an intermediate of the coenzyme biosynthesis pathway, are described. Methods for assessing the efficacy of treatment of, diagnosing, and prognosing sarcoma are also provided.

### RELATED APPLICATIONS

This application claims priority to U.S. Provisional Application Ser. No. 61/236,845, filed Aug. 25, 2009, entitled “Methods for Treatment of a Sarcoma Using an Epimetabolic Shifter (Coenzyme Q10)” (Attorney Docket No.: 117732-02601). The entire contents of the foregoing application is hereby incorporated herein by reference.

### BACKGROUND OF THE INVENTION

Cancer is presently one of the leading causes of death in developed nations and is a serious threat to modern society. Sarcomas, in particular, represent a heterogeneous group of malignancies of mesenchymal cell origin that develop at primary sites all over the body including the skeletal muscles, smooth muscle, bone and cartilage. Ewing's family of tumors (EFT) represents a family of morphologically small round cell malignant neoplasms including the classic Ewing Sarcoma (ES) of the bone, Extraosseus Ewing's (EOE), and the Primitive Neuroectodermal Tumors (PNET). They represent almost 3% of pediatric cancers and the second most common malignancy in children and adolescents. The frequency of Ewing Sarcoma is around 1-3 cases/million in the Western Hemisphere. Although considerable advances in the treatment of Ewing Sarcoma has increased the 5-year survival rates, the outcomes for Ewing patients with metastatic disease remains dire with less than 25% surviving beyond 5 years.

Ewing Sarcoma is a highly aggressive cancer incidence of which does not appear to be associated with Mendelian inheritance, environmental or drug exposure. The most consistent feature of Ewing Sarcoma is the presence of a fusion gene as a result of chromosomal translocation between the EWSR1 locus and the ETS transcription factor gene. The EWS-ETS fusion genes encode transcription factors such as EWS-FLI1, the aberrant functioning of which is associated with Ewing Sarcoma pathogenesis.

Although recent research has vastly increased our understanding of many of the molecular mechanisms of tumorigenesis and has provided numerous new avenues for the treatment of cancer, standard treatments for most malignancies, including Ewing's family of tumors, include gross ressection, chemotherapy, and radiotherapy. Each of these treatments may cause numerous undesired side effects. For example, surgery may result in pain, traumatic injury to healthy tissue, and scarring. Radiation therapy has the advantage of killing cancer cells but it also damages non-cancerous tissue at the same time. Chemotherapy involves the administration of various anti-cancer drugs to a patient. These standard treatments often are accompanied by adverse side effects, e.g., nausea, immune suppression, gastric ulceration and secondary tumorigenesis.

Over the years, many individuals and companies have conducted extensive research searching for improvements in the treatments for the wide array of cancers, including Ewing's family of tumors. Companies are developing bioactive agents including chemical entities, e.g., small molecules, and biologics, e.g., antibodies, with the desire of providing more beneficial therapies for cancer. For example, insulin-like growth factor receptor-1 (IGF-1R) antibodies are being investigated as potential therapy, alone and in combination with other standard chemotherapies, for the treatment of recurrent Ewing's family of tumors. To date, however, the Ewing's family of tumors remain very difficult to treat. Accordingly, there is a significant need for the development of novel therapies for the successful treatment of Ewing Sarcoma.

Coenzyme Q10, also referred to herein as CoQ10, Q10, ubiquinone, or ubidecarenone, is a popular nutritional supplement and can be found in capsule form in nutritional stores, health food stores, pharmacies, and the like, as a vitamin-like supplement to help protect the immune system through the antioxidant properties of ubiquinol, the reduced form of CoQ10. CoQ10 is art-recognized and further described in International Publication No. WO 2005/069916, the entire disclosure of which is incorporated by reference herein. Metabolism and function of CoQ10, including metabolites of CoQ10, are described in Turunen et al., Biochimica et Biophysica Acta 1660: 171-199 (2004), the entire contents of which are hereby incorporated herein by reference.

CoQ10 is found throughout most tissues of the human body and the tissues of other mammals. The tissue distribution and redox state of CoQ10 in humans has been reviewed in a review article by Bhagavan H N, et al., Coenzyme Q10: Absorption, tissue uptake, metabolism and pharmacokinetic, Free Radical Research 40(5), 445-453 (2006) (hereinafter, Bhagavan, et al.). The authors report that “as a general rule, tissues with high-energy requirements or metabolic activity such as the heart, kidney, liver and muscle contain relatively high concentrations of CoQ10.” The authors further report that “[a] major portion of CoQ10 in tissues is in the reduced form as the hydroquinone or uniquinol, with the exception of brain and lungs,” which “appears to be a reflection of increased oxidative stress in these two tissues.” In particular, Bhagavan et al. reports that in heart, kidney, liver, muscle, intenstine and blood (plasma), about 61%, 75%, 95%, 65%, 95% and 96%, respectively, of CoQ10 is in the reduced form. Similarly, Ruiz-Jiminez, et al., Determination of the ubiquinol-10 and ubiquinone-10 (coenzyme Q10) in human serum by liquid chromatography tandem mass spectrometry to evaluate the oxidative stress, J. Chroma A 1175(2), 242-248 (2007) (hereinafter Ruiz-Jiminez, et al.) reports that when human plasma was evaluated for Q10 and the reduced form of Q10 (Q10H2), the majority (90%) of the molecule was found in the reduced form.

CoQ10 is very lipophilic and, for the most part, insoluble in water. Due to its insolubility in water, limited solubility in lipids, and relatively large molecular weight, the efficiency of absorption of orally administered CoQ10 is poor. Bhagavan, et al. reports that “in one study with rats it was reported that only about 2-3% of orally-administered CoQ10 was absorbed.” Bhagavan, et al. further reports that “[d]ata from rat studies indicate that CoQ10 is reduced to ubiquinol either during or following absorption in the intestine.”

CoQ10 has been associated with cancer in the literature for many years. Described below are some representative but not all inclusive examples of the reported associations in the literature. Karl Folkers, et al., Survival of Cancer Patients on Therapy with Coenzyme Q10, Biochemical and Biophysical Research Communication 192, 241-245 (1993) (herein after “Folkers, et al.”) describes eight case histories of cancer patients “on therapy with CoQ10” and their stories of survival . . . “for periods of 5-15 years.” CoQ10 was orally administered to eight patients having different types of cancer, including pancreatic carcinoma, adenocarcinoma, laryngeal carcinoma, breast, colon, lung and prostate cancer. Folkers, et al. sets forth that “these results now justify systemic protocols.” Lockwood, et al., Progress on Therapy of Breast Cancer with Vitamin Q10 and the Regression of Metastases, Biochemical and Biophysical Research Communication 212, 172-177 (1995) (hereinafter “Lockwood, et al.”) is another review article that reports on the “[p]rogress on therapy of breast cancer with Vitamin Q10”. Lockwood, et al. refers to Folkers, et al., which “covers 35 years of international research on animals and humans which revealed variable levels of vitamin Q10 in non-tumor and tumor tissues and includes data on vitamin Q10 which are intrinsic to the host defense system as based on increased survivors of treated mice with tumors”. Lockwood, et al. further sets forth that “Mlle potential of vitamin Q10 therapy of human cancer became evident in 1961” relying on a study that determined the blood levels of CoQ10 in 199 Swedish and American cancer patients that revealed variable levels of deficiencies in cases of breast cancer. U.S. Pat. No. 6,417,233, issued Jul. 9, 2002 (hereinafter Sears, et al.) describes compositions containing lipid-soluble benzoquinones, e.g., coenzyme Q10, for the prevention and/or treatment of mitochondriopathies. Sears, et al. sets forth that “CoQ10 treatment has been reported to provide some benefits in cancer patients (see column 2, lines 30-31).”

As of the date of filing of this application, the National Cancer Institute reports that no well-designed clinical trials involving large numbers of patients of CoQ10 in cancer treatment have been conducted since “the way the studies were done and the amount of information reported made it unclear if the benefits were caused by the coenzyme Q10 or by something else.” See The National Cancer Institute (NCI), available at www.cancer.gov/cancertopics/pdq/cam/coenzymeQ10/patient/allpages (Sep. 29, 2008). In particular, the NCI cites three small studies on the use of CoQ10 as an adjuvant therapy after standard treatment in breast cancer patients, in which some patients appeared to be helped by the treatment, and reiterates that “weaknesses in study design and reporting, however, made it unclear if benefits were caused by the coenzyme Q10 or by something else.” The NCI specifies that “these studies had the following weaknesses: the studies were not randomized or controlled; the patients used other supplements in addition to coenzyme Q10; the patients received standard treatments before or during the coenzyme Q10 therapy; and details were not reported for all patients in the studies.” The NCI further reports on “anecdotal reports that coenzyme Q10 has helped some cancer patients live longer, including patients with cancers of the pancreas, lung, colon, rectum and prostate,” but states that ‘the patients described in these reports, however, also received treatments other than coenzyme Q10 including chemotherapy, radiation therapy and surgery.”

US Patent Application Publication 2006/0035981, published Feb. 16, 2006 (hereinafter “Mazzio 2006”) describes methods and formulations for treating or preventing human and animal cancers using compositions that exploit the vulnerability of cancers with regards to its anaerobic requirement for non-oxidative phosphorylation of glucose to derive energy, which is opposite to the host. The formulations of Mazzio 2006 contain one or more compounds that synergistically promote oxidative metabolism and/or impede lactic acid dehydrogenase or anaerobic glucose metabolism and more particularly are described as containing “2,3-dimethoxy-5-methyl-1,4-benzoquinone (herein also termed “DMBQ”) (quinoid base) and options for the entire ubiquinone series including corresponding hydroquinones, ubichromenols, ubichromanols or synthesized/natural derivatives and analogues. See Mazzio 2006 at page 3, paragraph 0010. Mazzio 2006 establishes “the short chain ubiquinones (CoQ<3) as anti-cancer agents and even further establishes that “2,3-dimethoxy-5-methyl-1,4-benzoquinone (DMBQ) is in excess of 1000 times more potent than CoQ10 as an anti-cancer agent.” See Mazzio 2006 at page 3, paragraph 0011. Mazzio 2006 further set forth that the study “did not find CoQ10 to be as lethal as expected” and like “previous studies that have employed CoQ10 against cancer have been somewhat contradictory”. See Mazzio 2006 at pages 3-4 for an extensive list of citations supporting this statement.

US Patent Application Publication 2007/0248693, published Oct. 25, 2007 (herein after “Mazzio 2007”) also describes nutraceutical compositions and their use for treating or preventing cancer. Again, this published patent application focuses on the short chain ubiquinones and specifically sets forth that CoQ10 is not a critical component of this invention. According to Mazzio 2007 “while CoQ10 can increase the Vmax of mitochondrial complex II activity in cancer cells (Mazzio and Soliman, Biochem Pharmacol. 67:1167-84, 2004), this did not control the rate of mitochondrial respiration or O2 utilization through complex IV. And, CoQ10 was not as lethal as expected. Likewise, results of CoQ10 against cancer have been contradictory.” See Mazzio 2007 at page 5, paragraph 0019.

Applicants have previously described topical formulations of CoQ10 and methods for reducing the rate of tumor growth in animal subjects (Hsia et al., WO 2005/069916 published Aug. 4, 2005). In the experiments described in Hsia et al., CoQ10 was shown to increase the rate of apoptosis in a culture of skin cancer cells but not normal cells. Moreover, treatment of tumor-bearing animals with a topical formulation of CoQ10 was shown to dramatically reduce the rate of tumor growth in the animals. The present invention is based, at least in part, upon a more complete understanding of the role of CoQ10 within a human and/or cell. In particular, the methods and formulations of the present invention are based, at least in part, upon the knowledge gained about the therapeutic mechanism of CoQ10 from extensive studies of CoQ10 treatment of sarcoma cells in vitro.

Specifically, in at least one embodiment, the methods and formulations of the present invention are based, at least in part, on the surprising discovery that the expression of a significant number of genes are modulated in primary sarcoma cells treated with CoQ10. These modulated proteins were found to be clustered into several cellular pathways, including regulation of cellular processes, metabolic processes, transcription regulation, programmed cell death (apoptosis), cell development, cytoskeleton, nucleus, proteosome and organ development. Taken together, the results described herein have provided insight into the therapeutic mechanism of Q10. While not wishing to be bound by theory, the results described herein suggest that Coenzyme Q10 induces global expression of cytoskeletal proteins, thereby destabilizing the cell's structural architecture and initiating a cellular program culminating in an unusually and unexpectedly rapid and robust apoptotic response.

Accordingly, the present invention provides, in one aspect, methods for treating or preventing a sarcoma in humans by topically administering a Coenzyme Q10 molecule (e.g., CoQ10, a building block of CoQ10, a derivative of CoQ10, an analog of CoQ10, a metabolite of CoQ10, or an intermediate of the coenzyme biosynthesis pathway) to the human such that treatment or prevention occurs. In an embodiment, the topical administration is via a dose selected for providing efficacy in humans for the particular sarcoma being treated. In certain embodiments, treatment or prevention of the sarcoma occurs by the administration of the oxidized form of Coenzyme Q10.

In certain embodiments, the sarcoma being treated or prevented is not a sarcoma that is typically treated or prevented by topical administration with the expectation of systemic delivery of an active agent in therapeutically effective levels.

In some embodiments, the concentration of the Coenzyme Q10 molecule in the tissues of the humans being treated is different than that of a control standard of human tissue representative of a healthy or normal state.

In certain other embodiments of the invention, the form of the Coenzyme Q10 molecule that is administered to the human is different than the predominant form found in systemic circulation within the human.

In another embodiment of the invention, the treatment involves or occurs via an interaction of a Coenzyme Q10 molecule (e.g., CoQ10, a building block of CoQ10, a derivative of CoQ10, an analog of CoQ10, a metabolite of CoQ10, or an intermediate of the coenzyme biosynthesis pathway) with a gene (or protein) selected from the group consisting of ANGPTL3, CCL2, CDH5, CXCL1, CXCL3, PRMT3, HDAC2, Nitric Oxide Synthase bNOS, Acetyl phospho Histone H3 AL9 S10, MTA 2, Glutamic Acid Decarboxylase GAD65 67, KSR, HDAC4, BOB1 OBF1, a1Syntrophin, BAP1, Importina 57, α E-Catenin, Grb2, Bax, Proteasome 26S subunit 13 (Endophilin B 1), Actin-like 6A (Eukaryotic Initiation Factor 4A11), Nuclear Chloride Channel protein, Proteasome 26S subunit, Dismutase Cu/Zn Superoxide, Translin-associated factor X, Arsenite translocating ATPase (Spermine synthetase), ribosomal protein SA, dCTP pyrophosphatase 1, proteasome beta 3, proteasome beta 4, acid phosphatase 1, diazepam binding inhibitor, alpha 2-HS glycoprotein (Bos Taurus, cow), ribosomal protein P2 (RPLP2); histone H2A, microtubule associated protein, proteasome alpha 3, eukaryotic translation elongation factor 1 delta, lamin B1, SMT 3 suppressor of mif two 3 homolog 2, heat shock protein 27 kD, hnRNP C1/C2, eukaryotc translation elongation factor 1 beta 2, Similar to HSPC-300, DNA directed DNA polymerase epislon 3; (canopy 2 homolog), LAMA5, PXLDC1, p300 CBP, P53R2, Phosphatidylserine Receptor, Cytokeratin Peptide 17, Cytokeratin peptide 13, Neurofilament 160 200, Rab5, Filensin, P53R2, MDM2, MSH6, Heat Shock Factor 2, AFX, FLIPg d, JAB 1, Myosine, MEKK4, cRaf pSer621, FKHR FOXO1a, MDM2, Fas Ligand, P53R2, Myosin Regulatory Light Chain, hnRNP C1/C2, Ubiquilin 1 (Phosphatase 2A), hnRNP C1/C2, alpha 2-HS glycoprotein (Bos Taurus, cow), beta actin, hnRNP C1/C2, heat shock protein 70 kD, beta tubulin, ATP dependent helicase II, eukaryotc translation elongation factor 1 beta 2, ER lipid raft associated 2 isoform 1 (beta actin), signal sequence receptor 1 delta, Eukaryotic translation initiation factor 3, subunit 3 gamma, Bilverdin reductase A (Transaldolase 1), Keratin 1,10 (Parathymosin), GST omega 1, chain B Dopamine Quinone Conjugation to Dj-1, Proteasome Activator Reg (alpha), T-complex protein 1 isoform A, Chain A Tapasin ERP57 (Chaperonin containing TCP1), Ubiquitin activating enzyme E1; Alanyl-tRNA synthetase, Dynactin 1, Heat shock protein 60 kd, Beta Actin, Spermidine synthase (Beta Actin), Heat Shock protein 70 kd, retinoblastoma binding protein 4 isoform A, TAR DNA binding protein, eukaryotic translation elongation factor 1 beta 2, chaperonin containing TCP1, subunit 3, cytoplasmic dynein IC-2, Angiotensin-converting enzyme (ACE), Caspase 3, GARS, Matrix Metalloproteinase 6 (MMP-6), Neurolysin (NLN)-Catalytic Domain, and Neurolysin (NLN), ADRB, CEACAM1, DUSP4, FOXC2, FOXP3, GCGR, GPD1, HMOX1, IL4R, INPPL1, IRS2, VEGFA, putative c-myc-responsive isoform 1, PDK 1, Caspase 12, Phospholipase D1, P34 cdc2, P53 BP1, BTK, ASC2, BUBR1, ARTS, PCAF, Raf1, MSK1, SNAP25, APRIL, DAPK, RAIDD, HAT1, PSF, HDAC1, Rad17, Surviving, SLIPR, MAG13, Caspase 10, Crk2, Cdc 6, P21 WAF 1 Cip 1, ASPP 1, HDAC 4, Cyclin B1, CD 40, GAD 65, TAP, Par4 (prostate apoptosis response 4), MRP1, MDC1, Laminin2 a2, bCatenin, FXR2, AnnexinV, SMAC Diablo, MBNL1, DImethyl Histone h3, Growth factor independence 1, U2AF65, mTOR, E2F2, Kaiso, Glycogen Synthase Kinase 3, ATF2, HDRP MITR, Neurabin I, AP1, and Apaf1.

In one embodiment, a Coenzyme Q10 molecule is administered at a dose that induces apoptosis in the cells of the sarcoma by at least 1 hour following the administration of said Coenzyme Q10 molecule to the human. In other embodiments, a Coenzyme Q10 molecule is administered at a dose that induces apoptosis in the sarcoma cells by at least about 2 hours, 3 hours, 4 hours, 5 hours, 6 hours, 7 hours, 8 hours, 9 hours, 10 hours, 12 hours, 15 hours, 18 hours, 24 hours, 36 hours, 48 hours following administration of Coenzyme Q10 to the human.

In certain embodiments of the invention, methods are provided for treating or preventing a sarcoma in a human by topically administering Coenzyme Q10 to the human such that treatment or prevention occurs, wherein the human is administered a topical dose of Coenzyme Q10 in a topical vehicle where Coenzyme Q10 is applied to the target tissue at a dose in the range of about 0.01 to about 0.5 milligrams of coenzyme Q10 per square centimeter of skin. In one embodiment, Coenzyme Q10 is applied to the target tissue at a dose in the range of about 0.09 to about 0.15 mg CoQ10 per square centimeter of skin. In another embodiment, Coenzyme Q10 is applied to the target tissue at a dose of about 0.12 milligrams of coenzyme Q10 per square centimeter of skin.

In certain embodiments of the invention, the sarcoma being treated or prevented is a type of sarcoma in Ewings' family of tumors. In certain embodiments, the type of sarcoma in Ewings' family of tumors that is being treated or prevented is Ewing's sarcoma.

Certain aspects of the invention provide methods for treating or preventing a sarcoma in a human by topically administering a Coenzyme Q10 molecule to the human such that treatment or prevention occurs, wherein the Coenzyme Q10 molecule is topically applied one or more times per 24 hours for six weeks or more.

In another aspect, the invention provides a method for treating or preventing asarcoma in a human, comprising administering Coenzyme Q10 to the human such that it is maintained in its oxidized form during treatment of the sarcoma. In one embodiment, the sarcoma being treated is not a sarcoma typically treated via topical administration, e.g., Ewing's sarcoma, with the expectation of systemic delivery of an active agent at therapeutically effective levels.

The present invention provides, in yet another aspect, methods for inhibiting the activity of the fusion protein generated by translocation between chromosome 11 and 22 found in Ewing's sarcoma, i.e., the EWS-FLI1 fusion protein. These methods include selecting or treating a human subject suffering from a sarcoma and administering to said human a therapeutically effective amount of a Coenzyme Q10 molecule, thereby inhibiting the activity of the EWS-FLI1 fusion protein.

In certain embodiments, the Coenzyme Q10 molecule is an intermediate in the CoQ10 biosynthesis pathway comprising: (a) benzoquinone or at least one molecule that facilitates the biosynthesis of the benzoquinone ring, and (b) at least one molecule that facilitates the synthesis of and/or attachment of isoprenoid units to the benzoquinone ring. In other embodiments, said at least one molecule which facilitates the biosynthesis of the benzoquinone ring comprises: L-Phenylalanine, DL-Phenylalanine, D-Phenylalanine, L-Tyrosine, DL-Tyrosine, D-Tyrosine, 4-hydroxy-phenylpyruvate, 3-methoxy-4-hydroxymandelate (vanillylmandelate or VMA), vanillic acid, pyridoxine, or panthenol. In other embodiments, said at least one molecule which facilitates the synthesis of and/or attachment of isoprenoid units to the benzoquinone ring comprises: phenylacetate, 4-hydroxy-benzoate, mevalonic acid, acetylglycine, acetyl-CoA, or farnesyl. In other embodiments, the intermediate comprises: (a) one or more of L-Phenylalanine, L-Tyrosine, and 4-hydroxyphenylpyruvate; and, (b) one or more of 4-hydroxy benzoate, phenylacetate, and benzoquinone. In other embodiments, the intermediate: (a) inhibits Bcl-2 expression and/or promotes Caspase-3 expression; and/or, (b) inhibits cell proliferation.

In another aspect, the invention provides a method for treating or preventing a sarcoma in a human. This method includes administering a Coenzyme Q10 molecule to a human in need thereof in a dosing regimen such that the permeability of the cell membranes of the human is modulated and treatment or prevention occurs.

In some embodiments, the methods for treating or preventing a sarcoma in a human or for inhibiting the activity of the EWS-FLI1 fusion protein in a human, further include upregulating the level of expression of one or more genes selected from the group consisting of LAMA5, PXLDC1, p300 CBP, P53R2, Phosphatidylserine Receptor, Cytokeratin Peptide 17, Cytokeratin peptide 13, Neurofilament 160 200, Rab5, Filensin, P53R2, MDM2, MSH6, Heat Shock Factor 2, AFX, FLIPg d, JAB 1, Myosine, MEKK4, cRaf pSer621, FKHR FOXO1a, MDM2, Fas Ligand, P53R2, Proteasome 26S subunit 13 (Endophilin B1), Myosin Regulatory Light Chain, hnRNP C1/C2, Ubiquilin 1 (Phosphatase 2A), hnRNP C1/C2, alpha 2-HS glycoprotein (Bos Taurus, cow), beta actin, hnRNP C1/C2, heat shock protein 70 kD, microtubule associated protein, beta tubulin, proteasome alpha 3, ATP dependent helicase II, eukaryotic translation elongation factor 1 delta, heat shock protein 27 kD, eukaryotc translation elongation factor 1 beta 2, Similar to HSPC-300, ER lipid raft associated 2 isoform 1 (beta actin), Dismutase Cu/Zn Superoxide, and signal sequence receptor 1 delta, ADRB, CEACAM1, DUSP4, FOXC2, FOXP3, GCGR, GPD1, HMOX1, IL4R, INPPL1, IRS2 and VEGFA, putative c-myc-responsive isoform 1, PDK 1, Caspase 12, Phospholipase D1, P34 cdc2, P53 BP1, BTK, ASC2, BUBR1, ARTS, PCAF, Raf1, MSK1, SNAP25, APRIL, DAPK, RAIDD, HAT1, PSF, HDAC1, Rad17, Surviving, SLIPR, MAG13, Caspase 10, Crk2, Cdc 6, P21 WAF 1 Cip 1, ASPP 1, HDAC 4, Cyclin B1, CD 40, GAD 65, TAP, Par4 (prostate apoptosis response 4), and MRP1, and/or downregulating the level of expression of one or more genes selected from the group consisting of ANGPTL3, CCL2, CDH5, CXCL1, CXCL3, PRMT3, HDAC2, Nitric Oxide Synthase bNOS, Acetyl phospho Histone H3 AL9 S10, MTA 2, Glutamic Acid Decarboxylase GAD65 67, KSR, HDAC4, BOB1 OBF1, a1Syntrophin, BAP1, Importina 57, α E-Catenin, Grb2, Bax, Proteasome 26S subunit 13 (Endophilin B 1), Actin-like 6A (Eukaryotic Initiation Factor 4A11), Nuclear Chloride Channel protein, Proteasome 26S subunit, Dismutase Cu/Zn Superoxide, Translin-associated factor X, Arsenite translocating ATPase (Spermine synthetase), ribosomal protein SA, dCTP pyrophosphatase 1, proteasome beta 3, proteasome beta 4, acid phosphatase 1, diazepam binding inhibitor, ribosomal protein P2 (RPLP2); histone H2A, microtubule associated protein, proteasome alpha 3, eukaryotic translation elongation factor 1 delta, lamin B 1, SMT 3 suppressor of mif two 3 homolog 2, heat shock protein 27 kD, hnRNP C1/C2, eukaryotc translation elongation factor 1 beta 2, Similar to HSPC-300, DNA directed DNA polymerase epislon 3 (canopy 2 homolog), Angiotensin-converting enzyme (ACE), Caspase 3, GARS, Matrix Metalloproteinase 6 (MMP-6), Neurolysin (NLN)-Catalytic Domain, Neurolysin (NLN), MDC1, Laminin2 a2, bCatenin, FXR2, AnnexinV, SMAC Diablo, MBNL1, DImethyl Histone h3, Growth factor independence 1, U2AF65, mTOR, E2F2, Kaiso, Glycogen Synthase Kinase 3, ATF2, HDRP MITR, Neurabin I, AP1, and Apaf1.

In some embodiments of the invention, the method for treating or preventing a sarcoma in a human or for inhibiting the activity of the EWS-FLI1 fusion protein in a human, involves or occurs via an interaction of a CoQ10 molecule with a gene (or protein) selected from the group consisting of ANGPTL3, CCL2, CDH5, CXCL1, CXCL3, PRMT3, HDAC2, Nitric Oxide Synthase bNOS, Acetyl phospho Histone H3 AL9 S10, MTA 2, Glutamic Acid Decarboxylase GAD65 67, KSR, HDAC4, BOB1 OBF1, a1Syntrophin, BAP1, Importina 57, α E-Catenin, Grb2, Bax, Proteasome 26S subunit 13 (Endophilin B 1), Actin-like 6A (Eukaryotic Initiation Factor 4A11), Nuclear Chloride Channel protein, Proteasome 26S subunit, Dismutase Cu/Zn Superoxide, Translin-associated factor X, Arsenite translocating ATPase (Spermine synthetase), ribosomal protein SA, dCTP pyrophosphatase 1, proteasome beta 3, proteasome beta 4, acid phosphatase 1, diazepam binding inhibitor, alpha 2-HS glycoprotein (Bos Taurus, cow), ribosomal protein P2 (RPLP2); histone H2A, microtubule associated protein, proteasome alpha 3, eukaryotic translation elongation factor 1 delta, lamin B1, SMT 3 suppressor of mif two 3 homolog 2, heat shock protein 27 kD, hnRNP C1/C2, eukaryotc translation elongation factor 1 beta 2, Similar to HSPC-300, DNA directed DNA polymerase epislon 3; (canopy 2 homolog), LAMA5, PXLDC1, p300 CBP, P53R2, Phosphatidylserine Receptor, Cytokeratin Peptide 17, Cytokeratin peptide 13, Neurofilament 160 200, Rab5, Filensin, P53R2, MDM2, MSH6, Heat Shock Factor 2, AFX, FLIPg d, JAB 1, Myosine, MEKK4, cRaf pSer621, FKHR FOXO1a, MDM2, Fas Ligand, P53R2, Myosin Regulatory Light Chain, hnRNP C1/C2, Ubiquilin 1 (Phosphatase 2A), hnRNP C1/C2, alpha 2-HS glycoprotein (Bos Taurus, cow), beta actin, hnRNP C1/C2, heat shock protein 70 kD, beta tubulin, ATP dependent helicase II, eukaryotc translation elongation factor 1 beta 2, ER lipid raft associated 2 isoform 1 (beta actin), signal sequence receptor 1 delta, Eukaryotic translation initiation factor 3, subunit 3 gamma, Bilverdin reductase A (Transaldolase 1), Keratin 1,10 (Parathymosin), GST omega 1, chain B Dopamine Quinone Conjugation to Dj-1, Proteasome Activator Reg (alpha), T-complex protein 1 isoform A, Chain A Tapasin ERP57 (Chaperonin containing TCP1), Ubiquitin activating enzyme E1; Alanyl-tRNA synthetase, Dynactin 1, Heat shock protein 60 kd, Beta Actin, Spermidine synthase (Beta Actin), Heat Shock protein 70 kd, retinoblastoma binding protein 4 isoform A, TAR DNA binding protein, eukaryotic translation elongation factor 1 beta 2, chaperonin containing TCP1, subunit 3, cytoplasmic dynein IC-2, Angiotensin-converting enzyme (ACE), Caspase 3, GARS, Matrix Metalloproteinase 6 (MMP-6), Neurolysin (NLN)-Catalytic Domain, and Neurolysin (NLN), ADRB, CEACAM1, DUSP4, FOXC2, FOXP3, GCGR, GPD1, HMOX1, IL4R, INPPL1, IRS2, VEGFA, putative c-myc-responsive isoform 1, PDK 1, Caspase 12, Phospholipase D1, P34 cdc2, P53 BP1, BTK, ASC2, BUBR1, ARTS, PCAF, Raf1, MSK1, SNAP25, APRIL, DAPK, RAIDD, HAT1, PSF, HDAC1, Rad17, Surviving, SLIPR, MAG13, Caspase 10, Crk2, Cdc 6, P21 WAF 1 Cip 1, ASPP 1, HDAC 4, Cyclin B1, CD 40, GAD 65, TAP, Par4 (prostate apoptosis response 4), MRP1, MDC1, Laminin2 a2, bCatenin, FXR2, AnnexinV, SMAC Diablo, MBNL1, DImethyl Histone h3, Growth factor independence 1, U2AF65, mTOR, E2F2, Kaiso, Glycogen Synthase Kinase 3, ATF2, HDRP MITR, Neurabin I, AN, and Apaf1.

In certain embodiments of the invention, the methods further include a treatment regimen which includes any one of or a combination of surgery, radiation, hormone therapy, antibody therapy, therapy with growth factors, cytokines, chemotherapy, and allogenic stem cell therapy. In yet another aspect, the invention provides methods of assessing the efficacy of a therapy for treating a sarcoma in a subject. The methods include comparing the level of expression of a marker present in a first sample obtained from the subject prior to administering at least a portion of the treatment regimen to the subject, wherein the marker is selected from the group consisting of the markers listed in Tables 2-9; and the level of expression of the marker present in a second sample obtained from the subject following administration of at least a portion of the treatment regimen, wherein a modulation in the level of expression of the marker in the second sample as compared to the first sample is an indication that the therapy is efficacious for treating the sarcoma in the subject.

In yet another aspect, the invention provides methods of assessing whether a subject is afflicted with a sarcoma. The methods include determining the level of expression of a marker present in a biological sample obtained from the subject, wherein the marker is selected from the group consisting of the markers listed in Tables 2-9, and comparing the level of expression of the marker present in the biological sample obtained from the subject with the level of expression of the marker present in a control sample, wherein a modulation in the level of expression of the marker in the biological sample obtained from the subject relative to the level of expression of the marker in the control sample is an indication that the subject is afflicted with the sarcoma, thereby assessing whether the subject is afflicted with the sarcoma.

In another aspect, the invention provides methods of prognosing whether a subject is predisposed to developing a sarcoma. The methods include determining the level of expression of a marker present in a biological sample obtained from the subject, wherein the marker is selected from the group consisting of the markers listed in Tables 2-9, and comparing the level of expression of the marker present in the biological sample obtained from the subject with the level of expression of the marker present in a control sample, wherein a modulation in the level of expression of the marker in the biological sample obtained from the subject relative to the level of expression of the marker in the control sample is an indication that the subject is predisposed to developing sarcoma, thereby prognosing whether the subject is predisposed to developing the sarcoma.

In yet another aspect, the invention provides methods of prognosing the recurrence of a sarcoma in a subject. The methods include determining the level of expression of a marker present in a biological sample obtained from the subject, wherein the marker is selected from the group consisting of the markers listed in Tables 2-9, and comparing the level of expression of the marker present in the biological sample obtained from the subject with the level of expression of the marker present in a control sample, wherein a modulation in the level of expression of the marker in the biological sample obtained from the subject relative to the level of expression of the marker in the control sample is an indication of the recurrence of the sarcoma, thereby prognosing the recurrence of the sarcoma in the subject.

In one aspect, the invention provides methods prognosing the survival of a subject with a sarcoma. The methods include determining the level of expression of a marker present in a biological sample obtained from the subject, wherein the marker is selected from the group consisting of the markers listed in Tables 2-9, and comparing the level of expression of the marker present in the biological sample obtained from the subject with the level of expression of the marker present in a control sample, wherein a modulation in the level of expression of the marker in the biological sample obtained from the subject relative to the level of expression of the marker in the control sample is an indication of survival of the subject, thereby prognosing survival of the subject with the sarcoma.

In yet another aspect, the invention provides methods of monitoring the progression of a sarcoma in a subject. The methods include comparing, the level of expression of a marker present in a first sample obtained from the subject prior to administering at least a portion of a treatment regimen to the subject and the level of expression of the marker present in a second sample obtained from the subject following administration of at least a portion of the treatment regimen, wherein the marker is selected from the group consisting of the markers listed in Tables 2-9, thereby monitoring the progression of the sarcoma in the subject.

In yet another aspect, the invention provides methods of identifying a compound for treating a sarcoma in a subject. The methods include obtaining a biological sample from the subject, contacting the biological sample with a test compound, determining the level of expression of one or more markers present in the biological sample obtained from the subject, wherein the marker is selected from the group consisting of the markers listed in Tables 2-9 with a positive fold change and/or with a negative fold change, comparing the level of expression of the one of more markers in the biological sample with an appropriate control, and selecting a test compound that decreases the level of expression of the one or more markers with a negative fold change present in the biological sample and/or increases the level of expression of the one or more markers with a positive fold change present in the biological sample, thereby identifying a compound for treating the sarcoma in a subject.

In one embodiment, the sarcoma is a type of sarcoma in Ewing's family of tumors. In one embodiment, the type of sarcoma is Ewing's sarcoma.

Suitable samples for use in the methods of the invention include, for example, a fluid, e.g., blood fluids, vomit, saliva, lymph, cystic fluid, urine, fluids collected by bronchial lavage, fluids collected by peritoneal rinsing, and gynecological fluids, obtained from the subject. In one embodiment, the sample is a blood sample or a component thereof. Suitable samples for use in the methods of the invention may also include, for example, a tissue or component thereof, e.g., bone, connective tissue, cartilage, lung, liver, kidney, muscle tissue, heart, pancreas, and/or skin.

In one embodiment, the subject is a human.

In one embodiment, the level of expression of the marker in the biological sample is determined by assaying a transcribed polynucleotide or a portion thereof by, e.g., amplifying the transcribed polynucleotide, in the sample.

In another embodiment, the level of expression of the marker in the subject sample is determined by assaying a protein or a portion thereof using, e.g., a reagent, e.g., a labeled reagent, which specifically binds with the protein in the sample. In one embodiment, the reagent is selected from the group consisting of an antibody and an antigen-binding antibody fragment.

In one embodiment, the level of expression of the marker in the sample is determined using a technique selected from the group consisting of polymerase chain reaction (PCR) amplification reaction, reverse-transcriptase PCR analysis, single-strand conformation polymorphism analysis (SSCP), mismatch cleavage detection, heteroduplex analysis, Southern blot analysis, Northern blot analysis, Western blot analysis, in situ hybridization, array analysis, deoxyribonucleic acid sequencing, restriction fragment length polymorphism analysis, and combinations or sub-combinations thereof, of said sample.

In another embodiment, the level of expression of the marker in the sample is determined using a technique selected from the group consisting of immunohistochemistry, immunocytochemistry, flow cytometry, ELISA and mass spectrometry.

In another embodiment, the marker is a marker selected from the group consisting of ANGPTL3, CCL2, CDH5, CXCL1, CXCL3, PRMT3, HDAC2, Nitric Oxide Synthase bNOS, Acetyl phospho Histone H3 AL9 S10, MTA 2, Glutamic Acid Decarboxylase GAD65 67, KSR, HDAC4, BOB1 OBF1, a1Syntrophin, BAP1, Importina 57, α E-Catenin, Grb2, Bax, Proteasome 26S subunit 13 (Endophilin B 1), Actin-like 6A (Eukaryotic Initiation Factor 4A11), Nuclear Chloride Channel protein, Proteasome 26S subunit, Dismutase Cu/Zn Superoxide, Translin-associated factor X, Arsenite translocating ATPase (Spermine synthetase), ribosomal protein SA, dCTP pyrophosphatase 1, proteasome beta 3, proteasome beta 4, acid phosphatase 1, diazepam binding inhibitor, alpha 2-HS glycoprotein (Bos Taurus, cow), ribosomal protein P2 (RPLP2); histone H2A, microtubule associated protein, proteasome alpha 3, eukaryotic translation elongation factor 1 delta, lamin B1, SMT 3 suppressor of mif two 3 homolog 2, heat shock protein 27 kD, hnRNP C1/C2, eukaryotc translation elongation factor 1 beta 2, Similar to HSPC-300, DNA directed DNA polymerase epislon 3; (canopy 2 homolog), LAMA5, PXLDC1, p300 CBP, P53R2, Phosphatidylserine Receptor, Cytokeratin Peptide 17, Cytokeratin peptide 13, Neurofilament 160 200, Rab5, Filensin, P53R2, MDM2, MSH6, Heat Shock Factor 2, AFX, FLIPg d, JAB 1, Myosine, MEKK4, cRaf pSer621, FKHR FOXO1a, MDM2, Fas Ligand, P53R2, Myosin Regulatory Light Chain, hnRNP C1/C2, Ubiquilin 1 (Phosphatase 2A), hnRNP C1/C2, alpha 2-HS glycoprotein (Bos Taurus, cow), beta actin, hnRNP C1/C2, heat shock protein 70 kD, beta tubulin, ATP dependent helicase II, eukaryotc translation elongation factor 1 beta 2, ER lipid raft associated 2 isoform 1 (beta actin), signal sequence receptor 1 delta, Eukaryotic translation initiation factor 3, subunit 3 gamma, Bilverdin reductase A (Transaldolase 1), Keratin 1,10 (Parathymosin), GST omega 1, chain B Dopamine Quinone Conjugation to Dj-1, Proteasome Activator Reg (alpha), T-complex protein 1 isoform A, Chain A Tapasin ERP57 (Chaperonin containing TCP1), Ubiquitin activating enzyme E1; Alanyl-tRNA synthetase, Dynactin 1, Heat shock protein 60 kd, Beta Actin, Spermidine synthase (Beta Actin), Heat Shock protein 70 kd, retinoblastoma binding protein 4 isoform A, TAR DNA binding protein, eukaryotic translation elongation factor 1 beta 2, chaperonin containing TCP1, subunit 3, cytoplasmic dynein IC-2, Angiotensin-converting enzyme (ACE), Caspase 3, GARS, Matrix Metalloproteinase 6 (MMP-6), Neurolysin (NLN)-Catalytic Domain, and Neurolysin (NLN), ADRB, CEACAM1, DUSP4, FOXC2, FOXP3, GCGR, GPD1, HMOX1, IL4R, INPPL1, IRS2, VEGFA, putative c-myc-responsive isoform 1, PDK 1, Caspase 12, Phospholipase D1, P34 cdc2, P53 BP1, BTK, ASC2, BUBR1, ARTS, PCAF, Raf1, MSK1, SNAP25, APRIL, DAPK, RAIDD, HAT1, PSF, HDAC1, Rad17, Surviving, SLIPR, MAG13, Caspase 10, Crk2, Cdc 6, P21 WAF 1 Cip 1, ASPP 1, HDAC 4, Cyclin B1, CD 40, GAD 65, TAP, Par4 (prostate apoptosis response 4), MRP1, MDC1, Laminin2 a2, bCatenin, FXR2, AnnexinV, SMAC Diablo, MBNL1, DImethyl Histone h3, Growth factor independence 1, U2AF65, mTOR, E2F2, Kaiso, Glycogen Synthase Kinase 3, ATF2, HDRP MITR, Neurabin I, AP1, and Apaf1.

In one embodiment, the level of expression of a plurality of markers is determined.

In one embodiment, the subject is being treated with a therapy selected from the group consisting of an environmental influencer compound, surgery, radiation, hormone therapy, antibody therapy, therapy with growth factors, cytokines, chemotherapy, and allogenic stem cell therapy.

In one embodiment, the therapy comprises an environmental influencer compound and, optionally, further comprises a treatment regimen selected from the group consisting of surgery, radiation, hormone therapy, antibody therapy, therapy with growth factors, cytokines, chemotherapy and allogenic stem cell therapy.

The environmental influencer compound may be a multidimensional intracellular molecule (MIM), an epimetabolic shifter (epi-shifter), a CoQ10 molecule, vitamin D3, acetyl Co-A, palmityl, L-carnitine, tyrosine, phenylalanine, cysteine, a small molecule, fibronectin, TNF-alpha, IL-5, IL-12, IL-23, an angiogenic factor and/or an apoptotic factor.

In yet another aspect of the invention, kit for assessing whether a subject is afflicted with a sarcoma are provided. The kits include reagents for determining the level of expression of at least one marker selected from the group consisting of the markers listed in Tables 2-9 and instructions for use of the kit to assess whether the subject is afflicted with the sarcoma.

In one aspect, the invention provides kits for prognosing whether a subject is predisposed to developing a sarcoma. The kits include reagents for determining the level of expression of at least one marker selected from the group consisting of the markers listed in Tables 2-9 and instructions for use of the kit to prognose whether the subject is predisposed to developing the sarcoma.

In another aspect, the invention provides kits for prognising the recurrence of a sarcoma in a subject. The kits include reagents for assessing the level of expression of at least one marker selected from the group consisting of the markers listed in Tables 2-9 and instructions for use of the kit to prognose the recurrence of the sarcoma.

In another aspect, the invention provides kits for prognising the recurrence of a sarcoma. The kits include reagents for determining the level of expression of at least one marker selected from the group consisting of the markers listed in Tables 2-9 and instructions for use of the kit to prognose the recurrence of the sarcoma.

In yet another aspect, the invention provides kits for prognising the survival of a subject with a sarcoma. The kits include reagents for determining the level of expression of at least one marker selected from the group consisting of the markers listed in Tables 2-9 and instructions for use of the kit to prognose the survival of the subject with the sarcoma.

In another aspect, the invention provides kits for monitoring the progression of a sarcoma in a subject. The kits include reagents for determining the level of expression of at least one marker selected from the group consisting of the markers listed in Tables 2-9 and instructions for use of the kit to prognose the progression of the sarcoma in a subject.

In yet another aspect, the invention provides kits for assessing the efficacy of a therapy for treating a sarcoma. The kits include reagents for determining the level of expression of at least one marker selected from the group consisting of the markers listed in Tables 2-9 and instructions for use of the kit to assess the efficacy of the therapy for treating the sarcoma.

The kits of the invention may further comprising means for obtaining a biological sample from a subject, a control sample, and/or an environmental influencer compound.

The means for determining the level of expression of at least one marker may comprise means for assaying a transcribed polynucleotide or a portion thereof in the sample and/or means for assaying a protein or a portion thereof in the sample.

In one embodiment, the kits comprise reagents for determining the level of expression of a plurality of markers.

### BRIEF DESCRIPTION OF THE DRAWINGS

Various embodiments of the present disclosure will be described herein below with reference to the figures wherein:

FIG. 1: Microscopy pictures of NCIES0808 cells from the different treatment groups. (A) 3 hours Media (B) 3 hours 50 uM Q10 (C) 3 hours 100 uM Q10 (D) 6 hours vehicle (E) 6 hours 50 uM Q10 (F) 6 hours 100 uM Q10 (G) 24 hours media (H) 24 hours 50 uM Q10 (I) 24 hours 100 uM Q10 (J) 48 hours media (K) 48 hours 50 uM Q10 (L) 48 hours 100 uM Q10 with no distinct differences in either cell number or morphology after Q10 treatment in any of the groups.

FIG. 2: Pattern analysis of exemplary antibody arrays of proteins isolated from NCIES0808 cells treated with 50 μM CoQ10 for 3 hours.

FIG. 3: Example gel analysis of 2-D gel electrophoresis of NCIES0808 cells treated with CoQ10 for 24 hours. Spots excised for identification are marked.

FIG. 4: Western blot analysis of proteins isolated from NCIES0808 cells treated with 50 uM or 100 uM CoQ10 for 24 hours using various antibodies. (A) Anti-Angiotensin-converting enzyme (ACE) (Santa Cruz Biotechnology, Inc., sc-23908). (B) Anti-Caspase 3 (abcam Inc., ab44976). (C) Anti-GARS (abcam Inc., ab42905). (D) Anti-Matrix Metalloproteinase 6 (MMP-6) (Santa Cruz Biotechnology, Inc., sc-101453). (E) Anti-Neurolysin (NON)—Catalytic Domain (abcam Inc., ab59523). (F) Anti-Neurolysin (NLN) (abcam Inc., ab59519).

FIG. 5: (A) Network of protein interactions for EWS and FLI1 proteins. (B) Network of protein interactions for ANGPTL3 protein.

### DETAILED DESCRIPTION OF THE INVENTION

In order that the present invention may be more readily understood, certain terms are first defined.

### I. Definitions

As used herein, each of the following terms has the meaning associated with it in this section.

The articles “a” and “an” are used herein to refer to one or to more than one (i.e. to at least one) of the grammatical object of the article. By way of example, “an element” means one element or more than one element.

The term “including” is used herein to mean, and is used interchangeably with, the phrase “including but not limited to”.

The term “or” is used herein to mean, and is used interchangeably with, the term “and/or,” unless context clearly indicates otherwise.

The term “such as” is used herein to mean, and is used interchangeably, with the phrase “such as but not limited to”.

A “patient” or “subject” to be treated by the method of the invention can mean either a human or non-human animal, preferably a mammal. It should be noted that clinical observations described herein were made with human subjects and, in at least some embodiments, the subjects are human.

“Therapeutically effective amount” means the amount of a compound that, when administered to a patient for treating a disease, is sufficient to effect such treatment for the disease. When administered for preventing a disease, the amount is sufficient to avoid or delay onset of the disease. The “therapeutically effective amount” will vary depending on the compound, the disease and its severity and the age, weight, etc., of the patient to be treated.

“Preventing” or “prevention” refers to a reduction in risk of acquiring a disease or disorder (i.e., causing at least one of the clinical symptoms of the disease not to develop in a patient that may be exposed to or predisposed to the disease but does not yet experience or display symptoms of the disease).

The term “prophylactic” or “therapeutic” treatment refers to administration to the subject of one or more of the subject compositions. If it is administered prior to clinical manifestation of the unwanted condition (e.g., disease or other unwanted state of the host animal) then the treatment is prophylactic, i.e., it protects the host against developing the unwanted condition, whereas if administered after manifestation of the unwanted condition, the treatment is therapeutic (i.e., it is intended to diminish, ameliorate or maintain the existing unwanted condition or side effects therefrom).

The term “therapeutic effect” refers to a local or systemic effect in animals, particularly mammals, and more particularly humans caused by a pharmacologically active substance. The term thus means any substance intended for use in the diagnosis, cure, mitigation, treatment or prevention of disease or in the enhancement of desirable physical or mental development and conditions in an animal or human. The phrase “therapeutically-effective amount” means that amount of such a substance that produces some desired local or systemic effect at a reasonable benefit/risk ratio applicable to any treatment. In certain embodiments, a therapeutically-effective amount of a compound will depend on its therapeutic index, solubility, and the like. For example, certain compounds discovered by the methods of the present invention may be administered in a sufficient amount to produce a reasonable benefit/risk ratio applicable to such treatment.

By “patient” is meant any animal (e.g., a human), including horses, dogs, cats, pigs, goats, rabbits, hamsters, monkeys, guinea pigs, rats, mice, lizards, snakes, sheep, cattle, fish, and birds.

“Metabolic pathway” refers to a sequence of enzyme-mediated reactions that transform one compound to another and provide intermediates and energy for cellular functions. The metabolic pathway can be linear or cyclic.

“Metabolic state” refers to the molecular content of a particular cellular, multicellular or tissue environment at a given point in time as measured by various chemical and biological indicators as they relate to a state of health or disease.

The term “microarray” refers to an array of distinct polynucleotides, oligonucleotides, polypeptides (e.g., antibodies) or peptides synthesized on a substrate, such as paper, nylon or other type of membrane, filter, chip, glass slide, or any other suitable solid support.

The terms “disorders” and “diseases” are used inclusively and refer to any deviation from the normal structure or function of any part, organ or system of the body (or any combination thereof). A specific disease is manifested by characteristic symptoms and signs, including biological, chemical and physical changes, and is often associated with a variety of other factors including, but not limited to, demographic, environmental, employment, genetic and medically historical factors. Certain characteristic signs, symptoms, and related factors can be quantitated through a variety of methods to yield important diagnostic information.

The term “sarcoma” refers to a malignant tumor of a tissue which connects, supports, or surrounds other structures and organs of the body. In one embodiment, a sarcoma is a type of sarcoma of the “Ewing's family of tumors.”

As used herein, the term “Ewing's family of tumors” is used interchangeably with the term “EFT” and refers to a group of cancers that affects the bones or nearby soft tissues. The term “Ewing's family of tumors” as used herein includes Ewing's tumor of the bones (also called Ewing's sarcoma), the most common type of EFT, Extraosseus Ewing's (EOE), a tumor that grows in soft tissues outside the bone, and Peripheral primitive neuroectodermal tumor (PPNET), a cancer found in the bones and soft tissues, including Askin's tumor, which is a PPNET of the chest wall.

The term “expression” is used herein to mean the process by which a polypeptide is produced from DNA. The process involves the transcription of the gene into mRNA and the translation of this mRNA into a polypeptide. Depending on the context in which used, “expression” may refer to the production of RNA, protein or both.

The terms “level of expression of a gene in a cell” or “gene expression level” refer to the level of mRNA, as well as pre-mRNA nascent transcript(s), transcript processing intermediates, mature mRNA(s) and degradation products, encoded by the gene in the cell.

The term “modulation” refers to upregulation (i.e., activation or stimulation), downregulation (i e, inhibition or suppression) of a response, or the two in combination or apart. A “modulator” is a compound or molecule that modulates, and may be, e.g., an agonist, antagonist, activator, stimulator, suppressor, or inhibitor.

A “higher level of expression”, “higher level of activity”, “increased level of expression” or “increased level of activity” refers to an expression level and/or activity in a test sample that is greater than the standard error of the assay employed to assess expression and/or activity, and is preferably at least twice, and more preferably three, four, five or ten or more times the expression level and/or activity of the marker in a control sample (e.g., a sample from a healthy subject not afflicted with sarcoma) and preferably, the average expression level and/or activity of the marker in several control samples.

A “lower level of expression”, “lower level of activity”, “decreased level of expression” or “decreased level of activity” refers to an expression level and/or activity in a test sample that is greater than the standard error of the assay employed to assess expression and/or activity, but is preferably at least twice, and more preferably three, four, five or ten or more times less than the expression level of the marker in a control sample (e.g., a sample that has been calibrated directly or indirectly against a panel of sarcomas with follow-up information which serve as a validation standard for prognostic ability of the marker) and preferably, the average expression level and/or activity of the marker in several control samples.

As used herein, “antibody” includes, by way of example, naturally-occurring forms of antibodies (e.g., IgG, IgA, IgM, IgE) and recombinant antibodies such as single-chain antibodies, chimeric and humanized antibodies and multi-specific antibodies, as well as fragments and derivatives of all of the foregoing, which fragments and derivatives have at least an antigenic binding site. Antibody derivatives may comprise a protein or chemical moiety conjugated to an antibody.

As used herein, “known standard” or “control” refers to one or more of an amount and/or mathematical relationship, as applicable, with regard to a marker of the invention, and the presence or absence of sarcoma. A known standard preferably reflects such amount and/or mathematical relationship characteristic of a recurrent tumor and a non-recurrent tumor and/or an aggressive or a non-aggressive tumor. Reagents for generating a known standard include, without limitation, tumor cells from a tumor known to be aggressive, tumor cells from a tumor known to be non-aggressive, and optionally labeled antibodies. Known standards may also include tissue culture cell lines (including, but not limited to, cell lines that have been manipulated to express specific marker proteins or to not express specific marker proteins, or tumor xenografts that either constitutively contain constant amounts of marker protein, or can be manipulated (e.g., by exposure to a changed environment, where such changed environment may include but not limited to growth factors, hormones, steroids, cytokines, antibodies, various drugs and anti-metabolites, and extracellular matrices) to express a marker protein. Cell lines may be mounted directly on glass slides for analysis, fixed, embedded in paraffin directly as a pellet, or suspended in a matrix such as agarose, then fixed, embedded in paraffin, sectioned and processed as tissue samples. The standards must be calibrated directly or indirectly against a panel of sarcomas with follow-up information which serve as a validation standard for prognostic ability of the marker proteins.

“Primary treatment” as used herein, refers to the initial treatment of a subject afflicted with sarcoma. Primary treatments include, without limitation, surgery, radiation, hormone therapy, chemotherapy, immunotherapy, angiogenic therapy, allogenic stem cell therapy, and therapy via biomodulators.

A sarcoma is “treated” if at least one symptom of the sarcoma is expected to be or is alleviated, terminated, slowed, or prevented. As used herein, sarcoma is also “treated” if recurrence or metastasis of the sarcoma is reduced, slowed, delayed, or prevented.

A kit is any manufacture (e.g. a package or container) comprising at least one reagent, e.g. a probe, for specifically detecting a marker of the invention, the manufacture being promoted, distributed, or sold as a unit for performing the methods of the present invention.

The term “Trolamine,” as used herein, refers to Trolamine NF, Triethanolamine, TEAlan®, TEAlan 99%, Triethanolamine, 99%, Triethanolamine, NF or Triethanolamine, 99%, NF. These terms may be used interchangeably herein.

A “Coenzyme Q10 molecule” or “CoQ10 molecule”, as used herein, includes Coenzyme Q10, a building block of CoQ10, a derivative of CoQ10, an analog of CoQ10, a metabolite of CoQ10, or an intermediate of the coenzyme biosynthesis pathway.

CoQ10 has the following structure:

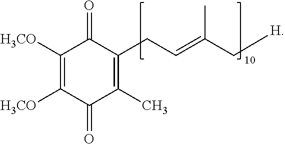

A “building block” of CoQ10 includes, but is not limited to, phenylalanine, tyrosine, 4-hydroxyphenylpyruvate, phenylacetate, 3-methoxy-4-hydroxymandelate, vanillic acid, 4-hydroxybenzoate, mevalonic acid, farnesyl, 2,3-dimethoxy-5-methyl-p-benzoquinone, as well as the corresponding acids or ions thereof.

A “derivative of CoQ10” is a compound that has a structure similar to CoQ10 except that one atom or functional group is replaced with another atom or group of atoms. An “analog of CoQ10” includes analogs having no or at least one (e.g., one, two, three, four, five, six, seven, eight, or nine) isoprenyl repeats.

The term “intermediate of the coenzyme biosynthesis pathway” as used herein, characterizes those compounds that are formed between the chemical/biological conversion of tyrosine and Acetyl-CoA to ubiquinone. Intermediates of the coenzyme biosynthesis pathway include 3-hexaprenyl-4-hydroxybenzoate, 3-hexaprenyl-4,5-dihydroxybenzoate, 3-hexaprenyl-4-hydroxy-5-methoxybenzoate, 2-hexaprenyl-6-methoxy-1,4-benzoquinone, 2-hexaprenyl-3-methyl-6-methoxy-1,4-benzoquinone, 2-hexaprenyl-3-methyl-5-hydroxy-6-methoxy-1,4-benzoquinone, 3-Octaprenyl-4-hydroxybenzoate, 2-octaprenylphenol, 2-octaprenyl-6-metholxyphenol, 2-octaprenyl-3-methyl-6-methoxy-1,4-benzoquinone, 2-octaprenyl-3-methyl-5-hydroxy-6-methoxy-1,4-benzoquinone, 2-decaprenyl-3-methyl-5-hydroxy-6-methoxy-1,4-benzoquinone, 2-decaprenyl-3-methyl-6-methoxy-1,4-benzoquinone, 2-decaprenyl-6-methoxy-1,4-benzoquinone, 2-decaprenyl-6-methoxyphenol, 3-decaprenyl-4-hydroxy-5-methoxybenzoate, 3-decaprenyl-4,5-dihydroxybenzoate, 3-decaprenyl-4-hydroxybenzoate, 4-hydroxy phenylpyruvate, 4-hydroxyphenyllactate, 4-hydroxy-benzoate, 4-hydroxycinnamate and hexaprenydiphosphate.

In certain embodiments, the intermediate of the coenzyme biosynthesis pathway comprises: (a) benzoquinone or at least one molecule that facilitates the biosynthesis of the benzoquinone ring, and (b) at least one molecule that facilitates the synthesis of and/or attachment of isoprenoid units to the benzoquinone ring. In other embodiments, said at least one molecule which facilitates the biosynthesis of the benzoquinone ring comprises: L-Phenylalanine, DL-Phenylalanine, D-Phenylalanine, L-Tyrosine, DL-Tyrosine, D-Tyrosine, 4-hydroxy-phenylpyruvate, 3-methoxy-4-hydroxymandelate (vanillylmandelate or VMA), vanillic acid, pyridoxine, or panthenol. In other embodiments, said at least one molecule which facilitates the synthesis of and/or attachment of isoprenoid units to the benzoquinone ring comprises: phenylacetate, 4-hydroxy-benzoate, mevalonic acid, acetylglycine, acetyl-CoA, or farnesyl. In other embodiments, the intermediate comprises: (a) one or more of L-Phenylalanine, L-Tyrosine, and 4-hydroxyphenylpyruvate; and, (b) one or more of 4-hydroxy benzoate, phenylacetate, and benzoquinone. In other embodiments, the intermediate: (a) inhibits Bcl-2 expression and/or promotes Caspase-3 expression; and/or, (b) inhibits cell proliferation.

In some embodiments, the compounds of the present invention, e.g., the MIMs or epi-shifters described herein, e.g., the Coenzyme Q10 molecules of the invention, share a common activity with Coenzyme Q10. As used herein, the phrase “share a common activity with Coenzyme Q10” refers to the ability of a compound to exhibit at least a portion of the same or similar activity as Coenzyme Q10. In some embodiments, the compounds of the present invention exhibit 25% or more of the activity of Coenzyme Q10. In some embodiments, the compounds of the present invention exhibit up to and including about 130% of the activity of Coenzyme Q10. In some embodiments, the compounds of the present invention exhibit about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 101%, 102%, 103%, 104%, 105%, 106%, 107%, 108%, 109%, 110%, 111%, 112%, 113%, 114%, 115%, 116%, 117%, 118%, 119%, 120%, 121%, 122%, 123%, 124%, 125%, 126%, 127%, 128%, 129%, or 130% of the activity of Coenzyme Q10. It is to be understood that each of the values listed in this paragraph may be modified by the term “about.” Additionally, it is to be understood that any range which is defined by any two values listed in this paragraph is meant to be encompassed by the present invention. For example, in some embodiments, the compounds of the present invention exhibit between about 50% and about 100% of the activity of Coenzyme Q10. In some embodiments, the activity shared by Coenzyme Q10 and the compounds of the present invention is the ability to induce a shift in cellular metabolism. In certain embodiments, the activity shared by of CoQ10 and the compounds of the present invention is measured by OCR (Oxigen Consumption Rate) and/or ECAR (ExtraCellular Acidification Rate). In certain embodiments, the activity shared by of CoQ10 and the compounds of the present invention is the ability to inhibit growth of a sarcoma cell. In certain embodiments, the activity shared by of CoQ10 and the compounds of the present invention is the ability to induce global expression of cytoskeletal proteins. In certain embodiments, the activity shared by of CoQ10 and the compounds of the present invention is the ability to destabilize the structural architecture of a cancer, e.g., sarcoma, cell.

Reference will now be made in detail to preferred embodiments of the invention. While the invention will be described in conjunction with the preferred embodiments, it will be understood that it is not intended to limit the invention to those preferred embodiments. To the contrary, it is intended to cover alternatives, modifications, and equivalents as may be included within the spirit and scope of the invention as defined by the appended claims.

### II. Environmental Influencers

In one aspect, the present invention provides methods of treating a sarcoma by administration of an Environmental influencer. “Environmental influencers” (Env-influencers) are molecules that influence or modulate the disease environment of a human in a beneficial manner allowing the human's disease environment to shift, reestablish back to or maintain a normal or healthy environment leading to a normal state. Env-influencers include both Multidimensional Intracellular Molecules (MIMs) and Epimetabolic shifters (Epi-shifters) as defined below. Env-influencers, MIMs and Epi-shifters are described in greater detail in U.S. patent application Ser. No. 12/778,094, U.S. patent application Ser. No. 12/777,902, U.S. patent application Ser. No. 12/778,029, U.S. patent application Ser. No. 12/778,054, and U.S. patent application Ser. No. 12/778,010, the entire contents of each of which are hereby incorporated herein by reference.

### 1. Multidimensional Intracellular Molecule (MIM)

The term “Multidimensional Intracellular Molecule (MIM)”, is an isolated version or synthetically produced version of an endogenous molecule that is naturally produced by the body and/or is present in at least one cell of a human. A MIM is capable of entering a cell and the entry into the cell includes complete or partial entry into the cell as long as the biologically active portion of the molecule wholly enters the cell. MIMs are capable of inducing a signal transduction and/or gene expression mechanism within a cell. MIMs are multidimensional because the molecules have both a therapeutic and a carrier, e.g., drug delivery, effect. MIMs also are multidimensional because the molecules act one way in a disease state and a different way in a normal state. For example, in the case of CoQ-10, administration of CoQ-10 to a melanoma cell in the presence of VEGF leads to a decreased level of Bcl2 which, in turn, leads to a decreased oncogenic potential for the melanoma cell. In contrast, in a normal fibroblast, co-administration of CoQ-10 and VEFG has no effect on the levels of Bcl2.

In one embodiment, a MIM is also an epi-shifter. In another embodiment, a MIM is not an epi-shifter. In another embodiment, a MIM is characterized by one or more of the foregoing functions. In another embodiment, a MIM is characterized by two or more of the foregoing functions. In a further embodiment, a MIM is characterized by three or more of the foregoing functions. In yet another embodiment, a MIM is characterized by all of the foregoing functions. The skilled artisan will appreciate that a MIM of the invention is also intended to encompass a mixture of two or more endogenous molecules, wherein the mixture is characterized by one or more of the foregoing functions. The endogenous molecules in the mixture are present at a ratio such that the mixture functions as a MIM.

MIMs can be lipid based or non-lipid based molecules. Examples of MIMs include, but are not limited to, CoQ10, acetyl Co-A, palmityl Co-A, L-carnitine, amino acids such as, for example, tyrosine, phenylalanine, and cysteine. In one embodiment, the MIM is a small molecule. In one embodiment of the invention, the MIM is not CoQ10. MIMs can be routinely identified by one of skill in the art using any of the assays described in detail herein.

### (i) Methods of Identifying MIMS

The present invention provides methods for identifying a MIM. Methods for identifying a MIM involve, generally, the exogenous addition to a cell of an endogenous molecule and evaluating the effect on the cell, e.g., the cellular microenvironment profile, that the endogenous molecule provides. Effects on the cell are evaluated at one or more of the cellular, mRNA, protein, lipid, and/or metabolite level to identify alterations in the cellular microenvironment profile. In one embodiment, the cells are cultured cells, e.g., in vitro. In one embodiment, the cells are present in an organism. The endogenous molecule may be added to the cell at a single concentration or may be added to the cell over a range of concentrations. In one embodiment, the endogenous molecule is added to the cells such that the level of the endogenous molecule in the cells is elevated (e.g., is elevated by 1.1 fold, 1.2 fold, 1.3 fold, 1.4 fold, 1.5 fold, 1.6 fold, 1.7 fold, 1.8 fold, 1.9 fold, 2.0 fold, 3.0 fold, 4.0 fold, 5.0 fold, 10 fold, 15 fold, 20 fold, 25 fold, 30 fold, 35 fold, 40 fold, 45 fold, 50 fold or greater) as compared to the level of the endogenous molecule in a control, untreated cell.

Molecules that induce a change in the cell as detected by alterations in, for example, any one or more of morphology, physiology, and/or composition (e.g., mRNA, protein, lipid, metabolite) may be evaluated further to determine if the induced changes to the cellular microenvironment profile are different between a disease cellular state and a normal cellular state. Cells (e.g., cell culture lines) of diverse tissue origin, cell type, or disease state may be evaluated for comparative evaluation. For example, changes induced in the cellular microenvironment profile of a cancer cell may be compared to changes induced to a non-cancerous or normal cell. An endogenous molecule that is observed to induce a change in the microenvironment profile of a cell (e.g., induces a change in the morphology, physiology and/or composition, e.g., mRNA, protein, lipid or metabolite, of the cell) and/or to differentially (e.g., preferentially) induce a change in the microenvironment profile of a diseased cell as compared to a normal cell, is identified as a MIM.

MIMs of the invention may be lipid based MIMs or non-lipid based MIMs. Methods for identifying lipid based MIMs involve the above-described cell based methods in which a lipid based endogenous molecule is exogenously added to the cell. In a preferred embodiment, the lipid based endogenous molecule is added to the cell such that the level of the lipid based endogenous molecule in the cell is elevated. In one embodiment, the level of the lipid based endogenous molecule is elevated by 1.1 fold, 1.2 fold, 1.3 fold, 1.4 fold, 1.5 fold, 1.6 fold, 1.7 fold, 1.8 fold, 1.9 fold, 2.0 fold, 3.0 fold, 4.0 fold, 5.0 fold, 10 fold, 15 fold, 20 fold, 25 fold, 30 fold, 35 fold, 40 fold, 45 fold, 50 fold or greater as compared to the level in an untreated control cell.

Formulation and delivery of the lipid based molecule to the cell is dependent upon the properties of each molecule tested, but many methods are known in the art. Examples of formulation and delivery of lipid based molecules include, but are not limited to, solubilization by co-solvents, carrier molecules, liposomes, dispersions, suspensions, nanoparticle dispersions, emulsions, e.g., oil-in-water or water-in-oil emulsions, multiphase emulsions, e.g., oil-in-water-in-oil emulsions, polymer entrapment and encapsulation. The delivery of the lipid based MIM to the cell can be confirmed by extraction of the cellular lipids and quantification of the MIM by routine methods known in the art, such as mass spectrometry.

Methods for identifying non-lipid based MIMs involve the above-described cell based methods in which a non-lipid based endogenous molecule is exogenously added to the cell. In a preferred embodiment, the non-lipid based endogenous molecule is added to the cell such that the level of the non-lipid based endogenous molecule in the cell is elevated. In one embodiment, the level of the non-lipid based endogenous molecule is elevated by 1.1 fold, 1.2 fold, 1.3 fold, 1.4 fold, 1.5 fold, 1.6 fold, 1.7 fold, 1.8 fold, 1.9 fold, 2.0 fold, 3.0 fold, 4.0 fold, 5.0 fold, 10 fold, 15 fold, 20 fold, 25 fold, 30 fold, 35 fold, 40 fold, 45 fold, 50 fold or greater as compared to the level in an untreated control cell. Formulation and delivery of the non-lipid based molecule to the cell is dependent upon the properties of each molecule tested, but many methods are known in the art. Examples of formulations and modes of delivery of non-lipid based molecules include, but are not limited to, solubilization by co-solvents, carrier molecules, active transport, polymer entrapment or adsorption, polymer grafting, liposomal encapsulation, and formulation with targeted delivery systems. The delivery of the non-lipid based MIM to the cell may be confirmed by extraction of the cellular content and quantification of the MIM by routine methods known in the art, such as mass spectrometry.

### 2. Epimetabolic Shifters (Epi-Shifters)

As used herein, an “epimetabolic shifter” (epi-shifter) is a molecule that modulates the metabolic shift from a healthy (or normal) state to a disease state and vice versa, thereby maintaining or reestablishing cellular, tissue, organ, system and/or host health in a human. Epi-shifters are capable of effectuating normalization in a tissue microenvironment. For example, an epi-shifter includes any molecule which is capable, when added to or depleted from a cell, of affecting the microenvironment (e.g., the metabolic state) of a cell. The skilled artisan will appreciate that an epi-shifter of the invention is also intended to encompass a mixture of two or more molecules, wherein the mixture is characterized by one or more of the foregoing functions. The molecules in the mixture are present at a ratio such that the mixture functions as an epi-shifter. Examples of epi-shifters include, but are not limited to, CoQ-10; vitamin D3; ECM components such as fibronectin; immunomodulators, such as TNFa or any of the interleukins, e.g., IL-5, IL-12, IL-23; angiogenic factors; and apoptotic factors.

In one embodiment, the epi-shifter also is a MIM. In one embodiment, the epi-shifter is not CoQ10. Epi-shifters can be routinely identified by one of skill in the art using any of the assays described in detail herein.

### (i) Methods of Identifying Epi-Shifters

Epimetabolic shifters (epi-shifter) are molecules capable of modulating the metabolic state of a cell, e.g., inducing a metabolic shift from a healthy (or normal) state to a disease state and vice versa, and are thereby capable of maintaining or reestablishing cellular, tissue, organ, system and/or host health in a human. Epi-shifters of the invention thus have utility in the diagnostic evaluation of a diseased state. Epi-shifters of the invention have further utility in therapeutic applications, wherein the application or administration of the epi-shifter (or modulation of the epi-shifter by other therapeutic molecules) effects a normalization in a tissue microenvironment and the disease state.

The identification of an epimetabolic shifter involves, generally, establishing a molecular profile, e.g., of metabolites, lipids, proteins or RNAs (as individual profiles or in combination), for a panel of cells or tissues that display differential disease states, progression, or aggressiveness. A molecule from the profile(s) for which a change in level (e.g., an increased or decreased level) correlates to the disease state, progression or aggressiveness is identified as a potential epi-shifter.

In one embodiment, an epi-shifter is also a MIM. Potential epi-shifters may be evaluated for their ability to enter cells upon exogenous addition to a cell by using any number of routine techniques known in the art, and by using any of the methods described herein. For example, entry of the potential epi-shifter into a cell may be confirmed by extraction of the cellular content and quantification of the potential epi-shifter by routine methods known in the art, such as mass spectrometry. A potential epi-shifter that is able to enter a cell is thereby identified as a MIM.

To identify an epi-shifter, a potential epi-shifter is next evaluated for the ability to shift the metabolic state of a cell. The ability of a potential epi-shifters to shift the metabolic state of the cell microenvironment is evaluated by introducing (e.g., exogenously adding) to a cell a potential epi-shifter and monitoring in the cell one or more of: changes in gene expression (e.g., changes in mRNA or protein expression), concentration changes in lipid or metabolite levels, changes in bioenergetic molecule levels, changes in cellular energetics, and/or changes in mitochondrial function or number. Potential epi-shifters capable of shifting the metabolic state of the cell microenvironment can be routinely identified by one of skill in the art using any of the assays described in detail herein. Potential epi-shifters are further evaluated for the ability to shift the metabolic state of a diseased cell towards a normal healthy state (or conversely, for the ability to shift the metabolic state of a normal cell towards a diseased state). A potential epi-shifter capable of shifting the metabolic state of a diseased cell towards a normal healthy state (or of shifting the metabolic state of healthy normal cell towards a diseased state) is thus identified as an Epi-shifter. In a preferred embodiment, the epi-shifter does not negatively impact the health and/or growth of normal cells.

Epimetabolic shifters of the invention include, but are not limited to, small molecule metabolites, lipid-based molecules, and proteins and RNAs. To identify an epimetabolic shifter in the class of small molecule endogenous metabolites, metabolite profiles for a panel of cells or tissues that display differential disease states, progression, or aggressiveness are established. The metabolite profile for each cell or tissue is determined by extracting metabolites from the cell or tissue and then identifying and quantifying the metabolites using routine methods known to the skilled artisan, including, for example, liquid-chromatography coupled mass spectrometry or gas-chromatography couple mass spectrometry methods. Metabolites for which a change in level (e.g., an increased or decreased level) correlates to the disease state, progression or aggressiveness, are identified as potential epi-shifters.

To identify epimetabolic shifters in the class of endogenous lipid-based molecules, lipid profiles for a panel of cells or tissues that display differential disease states, progression, or aggressiveness are established. The lipid profile for each cell or tissue is determined by using lipid extraction methods, followed by the identification and quantitation of the lipids using routine methods known to the skilled artisan, including, for example, liquid-chromatography coupled mass spectrometry or gas-chromatography couple mass spectrometry methods. Lipids for which a change in level (e.g., an increase or decrease in bulk or trace level) correlates to the disease state, progression or aggressiveness, are identified as potential epi-shifters.

To identify epimetabolic shifters in the class of proteins and RNAs, gene expression profiles for a panel of cells or tissues that display differential disease states, progression, or aggressiveness are established. The expression profile for each cell or tissue is determined at the mRNA and/or protein level(s) using standard proteomic, mRNA array, or genomic array methods, e.g., as described in detail herein. Genes for which a change in expression (e.g., an increase or decrease in expression at the mRNA or protein level) correlates to the disease state, progression or aggressiveness, are identified as potential epi-shifters.

Once the molecular profiles described above are established (e.g., for soluble metabolites, lipid-based molecules, proteins, RNAs, or other biological classes of composition), cellular and biochemical pathway analysis is carried out to elucidate known linkages between the identified potential epi-shifters in the cellular environment. This information obtained by such cellular and/or biochemical pathway analysis may be utilized to categorize the pathways and potential epi-shifters.

The utility of an Epi-shifter to modulate a disease state can be further evaluated and confirmed by one of skill in the art using any number of assays known in the art or described in detail herein. The utility of an Epi-shifter to modulate a disease state can be evaluated by direct exogenous delivery of the Epi-shifter to a cell or to an organism. The utility of an Epi-shifter to modulate a disease state can alternatively be evaluated by the development of molecules that directly modulate the Epi-shifter (e.g., the level or activity of the Epi-shifter). The utility of an Epi-shifter to modulate a disease state can also be evaluated by the development of molecules that indirectly modulate the Epi-shifter (e.g., the level or activity of the Epi-shifter) by regulating other molecules, such as genes (e.g., regulated at the RNA or protein level), placed in the same pathway as the Epi-shifter.

The Epimetabolomic approach described herein facilitates the identification of endogenous molecules that exist in a cellular microenvironment and the levels of which are sensed and controlled through genetic, mRNA, or protein-based mechanisms. The regulation response pathways found in normal cells that are triggered by an Epi-shifter of the invention may provide a therapeutic value in a misregulated or diseased cellular environment. In addition, the epimetabolic approach described herein identifies epi-shifters that may provide a diagnostic indication for use in clinical patient selection, a disease diagnostic kit, or as a prognostic indicator.

### III. Assays Useful for Identifying MIMs/Epi-Shifters

Techniques and methods of the present invention employed to separate and identify molecules and compounds of interest include but are not limited to: liquid chromatography (LC), high-pressure liquid chromatography (HPLC), mass spectroscopy (MS), gas chromatography (GC), liquid chromatography/mass spectroscopy (LC-MS), gas chromatography/mass spectroscopy (GC-MS), nuclear magnetic resonance (NMR), magnetic resonance imaging (MRI), Fourier Transform InfraRed (FT-IR), and inductively coupled plasma mass spectrometry (ICP-MS). It is further understood that mass spectrometry techniques include, but are not limited to, the use of magnetic-sector and double focusing instruments, transmission quadrapole instruments, quadrupole ion-trap instruments, time-of-flight instruments (TOF), Fourier transform ion cyclotron resonance instruments (FT-MS) and matrix-assisted laser desorption/ionization time-of-flight mass spectrometry (MALDI-TOF MS).

### Quantification of Bioenergetic Molecule Levels:

Environmental influencers (e.g., MIMs or Epi-shifters) may be identified by changes in cellular bioenergetic molecule levels (e.g., ATP, pyruvate, ADP, NADH, NAD, NADPH, NADP, acetylCoA, FADH2) of cells to which a candidate epi-shifter has been applied. Exemplary assays of bioenergetic molecule levels use colorometric, fluorescence, and/or bioluminescent-based methods. Examples of such assays are provided below.

Levels of ATP within cells can be measured with a number of assays and systems known in the art. For example, in one system, cytoplasmic ATP released from lysed cells reacts with luciferin and the enzyme luciferase to produce light. This bioluminescence is measured by a bioluminometer and the intracellular ATP concentration of the lysed cells can be calculated (EnzyLight™ ATP Assay Kit (EATP-100), BioAssay Systems, Hayward, Calif.). In another system, for example, both ATP and its dephosphorylated form, ADP, are calculated via bioluminescence; after ATP levels are calculated, ADP is transformed into ATP and then detected and calculated using the same luciferase system (ApoSENSOR™ ADP/ATP Ratio Assay Kit, BioVision Inc., Mountain View, Calif.).

Pyruvate is an important intermediate in cellular metabolic pathways. Pyruvate may be converted into carbohydrate via gluconeogenesis, converted into fatty acid or metabolized via acetyl CoA, or converted into alanine or ethanol, depending upon the metabolic state of a cell. Thus detection of pyruvate levels provides a measure of the metabolic activity and state of a cell sample. One assay to detect pyruvate, for example, uses both a colorimetric and fluorimetric to detect pyruvate concentrations within different ranges (EnzyChrom™ Pyruvate Assay Kit (Cat# EPYR-100), BioAssay Systems, Hayward, Calif.).

Environmental influencers (e.g., MIMs or Epi-shifters) may influence the process of oxidative phosphorylation carried out by mitochondria in cells, which are involved in the generation and maintenance of bioenergetic molecules in cells. In addition to assays that detect changes in cellular energetics in cell cultures and samples directly (described below), assays exist that detect and quantify the effects of compounds on discrete enzymes and complexes of mitochondria in cells. For example, the MT-OXC MitoTox™ Complete OXPHOS Activity Assay (MitoSciences Inc., Eugene, Oreg.) can detect and quantify the effects of compounds applied directly to complexes Ito V extracted from mitochondria. Assays for the detection and quantification of effects on individual mitochondrial complexes such as NADH dehydrogenase (Complex I), cytochrome c oxidase (Complex IV) and ATP synthase (Complex V) are also available (MitoSciences Inc., Eugene, Oreg.).

### Measurement of Cellular Energetics:

Environmental influencers (e.g., MIMs or Epi-shifters) may also be identified by changes in cellular energetics. One example of the measurement of cellular energetics are the real-time measures of the consumption of molecular oxygen and/or the change in pH of the media of a cell culture. For example, the ability of a potential epi-shifter to modulate the metabolic state of a cell may be analyzed using, for example, the XF24 Analyzer (Seahorse, Inc.). This technology allows for real time detection of oxygen and pH changes in a monolayer of cells in order to evaluate the bioenergetics of a cell microenvironment. The XF24 Analyzer measures and compares the rates of oxygen consumption (OCR), which is a measure of aerobic metabolism, and extracellular acidification (ECAR), which is a measure of glycolysis, both key indicators of cellular energetics.

### Measurement of Oxidative Phosphorylation and Mitochondrial Function

Oxidative Phosphorylation is a process by which ATP is generated via the oxidation of nutrient compounds, carried out in eukaryotes via protein complexes embedded in the membranes of mitochondria. As the primary source of ATP in the cells of most organisms, changes in oxidative phosphorylation activity can strongly alter metabolism and energy balance within a cell. In some embodiments of the invention, environmental influencers (e.g., MIMs or Epi-shifters) may be detected and/or identified by their effects on oxidative phosphorylation. In some embodiments, environmental influencers (e.g., MIMs or Epi-shifters) may be detected and/or identified by their effects on specific aspects of oxidative phosphorylation, including, but not limited to, the electron transport chain and ATP synthesis.

The membrane-embedded protein complexes of the mitochrondria that carry out processes involved in oxidative phosphorylation perform specific tasks and are numbered I, II, III and IV. These complexes, along with the trans-inner membrane ATP synthase (also known as Complex V), are the key entities involved in the oxidative phosphorylation process. In addition to assays that can examine the effects of environmental influencers (e.g., MIMs or Epi-shifters) on mitochondrial function in general and the oxidative phosphorylation process in particular, assays are available that can be used to examine the effects of an epi-shifter on an individual complex separately from other complexes.

Complex I, also known as NADH-coenzyme Q oxidoreductase or NADH dehydrogenase, is the first protein in the electron transport chain. In some embodiments, the detection and quantification of the effect of an epi-shifter on the production of NAD by Complex I may be performed. For example, the complex can be immunocaptured from a sample in a 96-well plate; the oxidation of NADH to NAD takes place concurrently with the reduction of a dye molecule which has an increased absorbance at 450 nM (Complex I Enzyme Activity Microplate Assay Kit, MitoSciences Inc., Eugene, Oreg.).

Complex IV, also known as cytochrome c oxidase (COX), is the last protein in the electron transport chain. In some embodiments, the detection and quantification of the effect of an epi-shifter on the oxidation of cytochrome c and the reduction of oxygen to water by Complex IV may be performed. For example, COX can be immunocaptured in a microwell plate and the oxidation of COX measured with a colorimetric assay (Complex IV Enzyme Activity Microplate Assay Kit, MitoSciences Inc., Eugene, Oreg.).

The final enzyme in the oxidative phosphorylation process is ATP synthase (Complex V), which uses the proton gradient created by the other complexes to power the synthesis of ATP from ADP. In some embodiments, the detection and quantification of the effect of an epi-shifter on the activity of ATP synthase may be performed. For example, both the activity of ATP synthase and the amount of ATP synthase in a sample may be measured for ATP synthase that has been immunocaptured in a microwell plate well. The enzyme can also function as an ATPase under certain conditions, thus in this assay for ATP synthase activity, the rate at which ATP is reduced to ADP is measured by detecting the simultaneous oxidation of NADH to NAD⁺. The amount of ATP is calculated using a labeled antibody to ATPase (ATP synthase Duplexing (Activity+Quantity) Microplate Assay Kit, MitoSciences Inc., Eugene, Oreg.). Additional assays for oxidative phosphorylation include assays that test for effects on the activity of Complexes II and III. For example, the MT-OXC MitoTox™ Complete OXPHOS System (MitoSciences Inc., Eugene, Oreg.) can be used to evaluate effects of a compound on Complex II and III as well as Complex I, IV and V, to provide data on the effects of a compound on the entire oxidative phosphorylation system.

As noted above, real-time observation of intact cell samples can be made using probes for changes in oxygen consumption and pH in cell culture media. These assays of cell energetics provide a broad overview of mitochondrial function and the effects of potential environmental influencers (e.g., MIMs or Epi-shifters) on the activity of mitochondria within the cells of the sample.

Environmental influencers (e.g., MIMs or Epi-shifters) may also affect mitochondrial permeability transition (MPT), a phenomena in which the mitochondrial membranes experience an increase in permeability due to the formation of mitochondrial permeability transition pores (MPTP). An increase in mitochondrial permeability can lead to mitochondrial swelling, an inability to conduct oxidative phosphorylation and ATP generation and cell death. MPT may be involved with induction of apoptosis. (See, for example, Halestrap, A. P., Biochem. Soc. Trans. 34:232-237 (2006) and Lena, A. et al. Journal of Translational Med. 7:13-26 (2009), hereby incorporated by reference in their entirety.)

In some embodiments, the detection and quantification of the effect of an environmental influencer (e.g., MIM or epi-shifter) on the formation, discontinuation and/or effects of MPT and MPTPs are measured. For example, assays can detect MPT through the use of specialized dye molecules (calcein) that are localized within the inner membranes of mitochondria and other cytosolic compartments. The application of another molecule, CoCl₂, serves to squelch the fluorescence of the calcein dye in the cytosol. CoCl₂ cannot access, however, the interior of the mitochondria, thus the calcein fluorescence in the mitochondria is not squelched unless MPT has occurred and CoCl₂ can access the interior of the mitochondria via MPTPs. Loss of mitochondrial-specific fluorescence signals that MPT has occurred. Flow cytometry can be used to evaluate cellular and organelle fluorescence (MitoProbe™ Transition Pore Assay Kit, Molecular Probes, Eugene, Oreg.). Additional assays utilize a fluorescence microscope for evaluating experimental results (Image-iT™ LIVE Mitochondrial Transition Pore Assay Kit, Molecular Probes, Eugene, Oreg.).

### Measurement of Cellular Proliferation and Inflammation

In some embodiments of the invention, environmental influencers (e.g., MIMs or Epi-shifters) may be identified and evaluated by their effects on the production or activity of molecules associated with cellular proliferation and/or inflammation. These molecules include, but are not limited to, cytokines, growth factors, hormones, components of the extra-cellular matrix, chemokines, neuropeptides, neurotransmitters, neurotrophins and other molecules involved in cellular signaling, as well as intracellular molecules, such as those involved in signal transduction.

Vascular endothelial growth factor (VEGF) is a growth factor with potent angiogenic, vasculogenic and mitogenic properties. VEGF stimulates endothelial permeability and swelling and VEGF activity is implicated in numerous diseases and disorders, including rheumatoid arthritis, metastatic cancer, age-related macular degeneration and diabetic retinopathy.

In some embodiments of the invention, an environmental influencer (e.g., MIM or Epi-shifter) may be identified and characterized by its effects on the production of VEGF. For example, cells maintained in hypoxic conditions or in conditions mimicking acidosis will exhibit increased VEGF production. VEGF secreted into media can be assayed using an ELISA or other antibody-based assays, using available anti-VEGF antibodies (R&D Systems, Minneapolis, Minn.). In some embodiments of the invention, an Epi-shifter may be identified and/or characterized based on its effect(s) on the responsiveness of cells to VEGF and/or based on its effect(s) on the expression or activity of the VEGF receptor.

Implicated in both healthy immune system function as well as in autoimmune diseases, tumor necrosis factor (TNF) is a key mediator of inflammation and immune system activation. In some embodiments of the invention, an Epi-shifter may be identified and characterized by its effects on the production or the activity of TNF. For example, TNF produced by cultured cells and secreted into media can be quantified via ELISA and other antibody-based assays known in the art. Furthermore, in some embodiments an environmental influencer may be identified and characterized by its effect(s) on the expression of receptors for TNF (Human TNF RI Duoset, R&D Systems, Minneapolis, Minn.).

The components of the extracellular matrix (ECM) play roles in both the structure of cells and tissues and in signaling processes. For example, latent transforming growth factor beta binding proteins are ECM components that create a reservoir of transforming growth factor beta (TGFβ) within the ECM. Matrix-bound TGFβ can be released later during the process of matrix remodeling and can exert growth factor effects on nearby cells (Dallas, S. Methods in Mol. Biol. 139:231-243 (2000)).

In some embodiments, an environmental influencer (e.g., MIM or Epi-shifter) may be identified or characterized by its effect(s) on the creation of ECM by cultured cells. Researchers have developed techniques with which the creation of ECM by cells, as well as the composition of the ECM, can be studied and quantified. For example, the synthesis of ECM by cells can be evaluated by embedding the cells in a hydrogel before incubation. Biochemical and other analyses are performed on the ECM generated by the cells after cell harvest and digestion of the hydrogel (Strehin, I. and Elisseeff, J. Methods in Mol. Bio. 522:349-362 (2009)).

In some embodiments, the effect of environmental influencer (e.g., MIM or epi-shifter) on the production, status of or lack of ECM or one of its components in an organism may be identified or characterized. Techniques for creating conditional knock-out (KO) mice have been developed that allow for the knockout of particular ECM genes only in discrete types of cells or at certain stages of development (Brancaccio, M. et al. Methods in Mol Bio. 522:15-50 (2009)). The effect of the application or administration of an epi-shifter or potential epi-shifter on the activity or absence of a particular ECM component in a particular tissue or at a particular stage of development may thus be evaluated.

### Measurement of Plasma Membrane Integrity and Cell Death

Environmental influencers (e.g., MIMs or Epi-shifters) may be identified by changes in the plasma membrane integrity of a cell sample and/or by changes in the number or percentage of cells that undergo apoptosis, necrosis or cellular changes that demonstrate an increased or reduced likelihood of cell death.

An assay for lactate dehydrogenase (LDH) can provide a measurement of cellular status and damage levels. LDH is a stable and relatively abundant cytoplasmic enzyme. When plasma membranes lose physical integrity, LDH escapes to the extracellular compartment. Higher concentrations of LDH correlate with higher levels of plasma membrane damage and cell death. Examples of LDH assays include assays that use a colorimetric system to detect and quantify levels of LDH in a sample, wherein the reduced form of a tetrazolium salt is produced via the activity of the LDH enzyme (QuantiChrom™ Lactate Dehydrogenase Kit (DLDH-100), BioAssay Systems, Hayward, Calif.; LDH Cytotoxicity Detection Kit, Clontech, Mountain View, Calif.).

Apoptosis is a process of programmed cell death that may have a variety of different initiating events. A number of assays can detect changes in the rate and/or number of cells that undergo apoptosis. One type of assay that is used to detect and quantify apoptosis is a capase assay. Capases are aspartic acid-specific cysteine proteases that are activated via proteolytic cleavage during apoptosis. Examples of assays that detect activated capases include PhiPhiLux® (OncoImmunin, Inc., Gaithersburg, Md.) and Caspase-Glo® 3/7 Assay Systems (Promega Corp., Madison, Wis.). Additional assays that can detect apoptosis and changes in the percentage or number of cells undergoing apoptosis in comparative samples include TUNEL/DNA fragmentation assays. These assays detect the 180 to 200 base pair DNA fragments generated by nucleases during the execution phase of apoptosis. Exemplary TUNEL/DNA fragmentation assays include the In Situ Cell Death Detection Kit (Roche Applied Science, Indianapolis, Ind.) and the DeadEnd™ Colorimetric and Fluorometric TUNEL Systems (Promega Corp., Madison, Wis.).

Some apoptosis assays detect and quantify proteins associated with an apoptotic and/or a non-apoptotic state. For example, the MultiTox-Fluor Multiplex Cytotoxicity Assay (Promega Corp., Madison, Wis.) uses a single substrate, fluorimetric system to detect and quantify proteases specific to live and dead cells, thus providing a ratio of living cells to cells that have undergone apoptosis in a cell or tissue sample.

Additional assays available for detecting and quantifying apoptosis include assays that detect cell permeability (e.g., APOPercentage™ APOPTOSIS Assay, Biocolor, UK) and assays for Annexin V (e.g., Annexin V-Biotin Apoptosis Detection Kit, BioVision Inc., Mountain View, Calif.).

### IV. Treatment of a Sarcoma

The present invention provides methods of treating or preventing a sarcoma in a human, comprising administering an environmental influencer, e.g., a MIM or EPI shifter, e.g., a CoQ10 molecule (e.g., CoQ10, a building block of CoQ10, a derivative of CoQ10, an analog of CoQ10, a metabolite of CoQ10, or an intermediate of the coenzyme biosynthesis pathway) to the human in an amount sufficient to treat or prevent the sarcoma, thereby treating or preventing the sarcoma. In a preferred embodiment, the methods of treating or preventing a sarcoma in a human comprise administering a CoQ10 molecule to the human in an amount sufficient to treat or prevent the sarcoma, thereby treating or preventing the sarcoma.

The present invention also provides compositions of a CoQ10 molecule and methods of preparing same. In one embodiment, the present invention provides CoQ10 compositions and methods of preparing the same. Preferably, the compositions comprise at least about 1% to about 25% CoQ10 w/w. CoQ10 can be obtained from Asahi Kasei N&P (Hokkaido, Japan) as UBIDECARENONE (USP). CoQ10 can also be obtained from Kaneka Q10 as Kaneka Q10 (USP UBIDECARENONE) in powdered form (Pasadena, Tex., USA). CoQ10 used in the methods exemplified herein have the following characteristics: residual solvents meet USP 467 requirement; water content is less than 0.0%, less than 0.05% or less than 0.2%; residue on ignition is 0.0%, less than 0.05%, or less than 0.2% less than; heavy metal content is less than 0.002%, or less than 0.001%; purity of between 98-100% or 99.9%, or 99.5%. Methods of preparing the compositions are provided herein.

As used herein, the terms or language “oncological disorder”, “cancer,” “neoplasm,” and “tumor,” are used interchangeably and in either the singular or plural form, refer to cells that have undergone a malignant transformation that makes them pathological to the host organism. Primary cancer cells (that is, cells obtained from near the site of malignant transformation) can be readily distinguished from non-cancerous cells by well-established techniques, particularly histological examination. The definition of a cancer cell, as used herein, includes not only a primary cancer cell, but also cancer stem cells, as well as cancer progenitor cells or any cell derived from a cancer cell ancestor. This includes metastasized cancer cells, and in vitro cultures and cell lines derived from cancer cells. When referring to a type of cancer that normally manifests as a solid tumor, a “clinically detectable” tumor is one that is detectable on the basis of tumor mass; e.g., by procedures such as CAT scan, MR imaging, X-ray, ultrasound or palpation, and/or which is detectable because of the expression of one or more cancer-specific antigens in a sample obtainable from a patient.

The term “sarcoma” generally refers to a tumor which is made up of a substance like the embryonic connective tissue and is generally composed of closely packed cells embedded in a fibrillar or homogeneous substance. Examples of sarcomas which can be treated with an environmental influencer of the invention include, but are not limited to, Ewing's family of tumors (e.g., Ewing's sarcoma (also referred to as Ewing's tumor of the bones), Extraosseus Ewing's (EOE), and Peripheral primitive neuroectodermal tumor (PPNET)), a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

Accordingly, in one embodiment, the methods of treatment or prevention of the invention involve the treatment or prevention of a sarcoma selected from the group consisting of Ewing's sarcoma, Extraosseus Ewing's (EOE), Peripheral primitive neuroectodermal tumor (PPNET) and Askin's tumor. In one embodiment, the sarcoma is Ewing's sarcoma. In one embodiment, the sarcoma is EOE. In one embodiment, the sarcoma is PPNET. In one embodiment, the sarcoma is Askin's tumor.

In some embodiments, the sarcoma is characterized by a lack of apoptosis. In other embodiments, the sarcoma is characterized by increased angiogenesis. In other embodiments, the sarcoma is characterized by extracellular matrix (ECM) degradation. In yet other embodiments, the sarcoma is characterized by loss of cell cycle control. In still other embodiments, the sarcoma is characterized by a shift in metabolic governance from mitochondrial oxidative phosphorylation to increased utilization and/or dependency on lactate and glycolytic flux. In further embodiments, the sarcoma is characterized by adapted immunomodulatory mechanisms that have evaded immunosurveilance. In one embodiment, the sarcoma is characterized by at least two of the above features, e.g., increased angiogenesis and ECM degradation. In one embodiment, the sarcoma is characterized by at least three of the above features. In one embodiment, the sarcoma is characterized by at least four of the above features. In one embodiment, the sarcoma is characterized by at least five of the above features. In one embodiment, the sarcoma is characterized by all six of the above features.

Accordingly, in some embodiments, the CoQ10 molecules of the present invention function by restoring the capacity for apoptosis or inducing apoptosis. In other embodiments, the CoQ10 molecules of the present invention function by reducing, decreasing or inhibiting angiogenesis. In still other embodiments, the CoQ10 molecules of the present invention function by restoring re-establishing extracellular matrix. In other embodiments, the CoQ10 molecules of the present invention function by restoring cell cycle control. In still other embodiments, the CoQ10 molecules of the present invention function by shifting metabolic governance back from glycolysis to mitochondrial oxidative phosphorylation. In further embodiments, the CoQ10 molecules of the present invention function by restoring immunosurveilance or restoring the body's ability to recognize the cancer cell as foreign.

Without wishing to be bound by any particular theory, it is believed that there is typically a coordinated cascade of events that aggregate to develop into cancer, e.g., a sarcoma. That is, in some embodiments, cancer, such as a sarcoma is not singularly dependent on a 1 gene-1 protein-root causality. In some embodiments, cancer, such as a sarcoma, is a physiologic disease state that manifests into tissue changes and alterations that become tumors, altered tissue states, e.g., energetics, compromised extracellular matrix integrity that allows for metastatic potential, lack of immunosurveilance and/or altered state of angiogenesis.

Primary cancer cells, e.g., primary sarcoma cells (that is, cells obtained from near the site of malignant transformation) can be readily distinguished from non-cancerous cells by well-established techniques, particularly histological examination. The definition of a cancer cell, as used herein, includes not only a primary cancer cell, but also cancer stem cells, as well as cancer progenitor cells or any cell derived from a cancer cell ancestor. This includes metastasized cancer cells, and in vitro cultures and cell lines derived from cancer cells. When referring to a type of cancer that normally manifests as a solid tumor, a “clinically detectable” tumor is one that is detectable on the basis of tumor mass; e.g., by procedures such as CAT scan, MR imaging, X-ray, ultrasound or palpation, and/or which is detectable because of the expression of one or more cancer-specific antigens in a sample obtainable from a patient.

In some embodiments, the compounds of the present invention, e.g., the Coenzyme Q10 molecules of the invention, may be used to treat a Coenzyme Q10 responsive sarcoma in a subject in need thereof. The language “Coenzyme Q10 responsive sarcoma,” or “CoQ10 responsive sarcoma,” includes sarcomas which can be treated, prevented, or otherwise ameliorated by the administration of Coenzyme Q10. Without wishing to be bound by any particular theory, and as described further herein, it is believed that CoQ10 functions, at least partially, by inducing a metabolic shift to the cell microenvironment, such as a shift towards the type and/or level of oxidative phosphorylation in normal state cells. Accordingly, in some embodiments, CoQ10 responsive sarcomas are sarcomas that arise from an altered metabolism of cell microenvironment. Coenzyme Q10 responsive sarcomas include, for example, sarcomas, which, for example, may be biased towards glycolysis and lactate biosynthesis.

In general, a CoQ10 molecule (e.g., CoQ10, a building block of CoQ10, a derivative of CoQ10, an analog of CoQ10, a metabolite of CoQ10, or an intermediate of the coenzyme biosynthesis pathway) may be used to prophylactically or therapeutically treat any neoplasm. In one embodiment, a CoQ10 molecule is used to treat or prevent a sarcoma. In one embodiment, a CoQ10 molecule is used for treatment of a Ewing's family of tumors. In one embodiment, the Ewing's family of tumors is Ewing's sarcoma.

The definition of a cancer cell, as used herein, is intended to include a cancer cell that produces energy by anaerobic glycolysis (e.g., glycolysis followed by lactic acid fermantion in the cytosol), aerobic glycolysis (e.g., glycolysis followed by oxidation of pyruvate in the mitochondria), or a combination of anaerobic glycolysis and aerobic glycolysis. In one embodiment, a cancer cell produces energy predominantly by anaerobic glycolysis (e.g., at least 50%, 60%, 70%, 80%, 90%, 95% or more of the cell's energy is produced by anaerobic glycolysis). In one embodiment, a cancer cell produces energy predominantly by aerobic glycolysis (e.g., at least 50%, 60%, 70%, 80%, 90%, 95% or more of the cell's energy is produced by anaerobic glycolysis). The definition of cancer cells, as used herein, is also intended to include a cancer cell population or mixture of cancer cells comprising cells that produce energy by anaerobic glycolysis and cells that produce energy by aerobic glycolysis. In one embodiment, a cancer cell population comprises predominantly cells that produce energy by anaerobic glycolysis (e.g., at least 50%, 60%, 70%, 80%, 90%, 95% or more of the cells in the population produce energy by anaerobic glycolysis). In one embodiment, a cancer cell population comprises predominantly cells that produce energy by aerobic glycolysis (e.g., at least 50%, 60%, 70%, 80%, 90%, 95% or more of the cells in the population).

As used herein, the phrase “anaerobic use of glucose” or “anaerobic glycolysis” refers to cellular production of energy by glycolysis followed by lactic acid fermentation in the cytosol. For example, many cancer cells produce energy by anaerobic glycolysis.

As used herein, the phrase “aerobic glycolysis” or “mitochondrial oxidative phosphorylation” refers to cellular production of energy by glycolysis followed by oxidation of pyruvate in mitochondria.

As used herein, the phrase “capable of blocking anaerobic use of glucose and augmenting mitochondrial oxidative phosphorylation” refers to the ability of an environmental influencer (e.g., an epitmetabolic shifter) to induce a shift or change in the metabolic state of a cell from anaerobic glycolysis to aerobic glycolysis or mitochondrial oxidative phosphorylation.

In some embodiments of the invention, the sarcoma being treated is not a disorder typically treated via topical administration with the expectation of systemic delivery of an active agent at therapeutically effective levels. As used herein, the phrase “not a disorder typically treated via topical administration” refers to sarcomas that are not typically or routinely treated with a therapeutic agent via topical administration but rather are typically treated with a therapeutic agent via, for example, intravenous administration.

The present invention also provides a method for treating or preventing an aggressive oncological disorder in a human, comprising administering a CoQ10 molecule (e.g., CoQ10, a building block of CoQ10, a derivative of CoQ10, an analog of CoQ10, a metabolite of CoQ10, or an intermediate of the coenzyme biosynthesis pathway) to the human at a selected lower dose than the dosage regimen used or selected for less aggressive or non-aggressive oncological disorders, thereby treating or preventing the aggressive oncological disorder. In a related aspect, the invention provides a method for treating or preventing a non-aggressive oncological disorder in a human, comprising administering an environmental influencer to the human at a selected higher dose over the dosage regimen used or selected for aggressive oncological disorders, thereby treating or preventing the non-aggressive oncological disorder.

As used herein, the term “aggressive oncological disorder” refers to an oncological disorder involving a fast-growing tumor. An aggressive oncological disorder typically does not respond or responds poorly to therapeutic treatment. Examples of an aggressive oncological disorder include, but are not limited to, pancreatic carcinoma, hepatocellular carcinoma, Ewing's sarcoma, metastatic breast cancer, metastatic melanoma, brain cancer (astrocytoma, glioblastoma), neuroendocrine cancer, colon cancer, lung cancer, osteosarcoma, androgen-independent prostate cancer, ovarian cancer and non-Hodgkin's Lymphoma.

As used herein, the term “non-aggressive oncological disorder” refers to an oncological disorder involving a slow-growing tumor. A non-aggressive oncological disorder typically responds favorably or moderately to therapeutic treatment. Examples of a non-aggressive oncological disorder include, but are not limited to, non-metastatic breast cancer, androgen-dependent prostate cancer, small cell lung cancer and acute lymphocytic leukemia. In one embodiment, non-aggressive oncological disorders include any oncological disorder that is not an aggressive oncological disorder.

The present invention also provides a method for disrupting cytoskeletal architecture in sarcoma cells of a human, comprising selecting a human subject suffering from sarcoma, and administering to said human a therapeutically effective amount of a Coenzyme Q10 molecule (e.g., CoQ10, a building block of CoQ10, a derivative of CoQ10, an analog of CoQ10, a metabolite of CoQ10, or an intermediate of the coenzyme biosynthesis pathway), thereby disrupting the cytoskeletal architecture of sarcoma cells in the human. In one embodiment, this method involves the upregulation of expression of one or more cytoskeletal genes or proteins.

In one embodiment, a CoQ10 molecule (e.g., CoQ10, a building block of CoQ10, a derivative of CoQ10, an analog of CoQ10, a metabolite of CoQ10, or an intermediate of the coenzyme biosynthesis pathway) reduces tumor size, inhibits tumor growth and/or prolongs the survival time of a tumor-bearing subject. Accordingly, this invention also relates to a method of treating tumors in a human or other animal by administering to such human or animal an effective, non-toxic amount of a CoQ10 molecule (e.g., CoQ10, a building block of CoQ10, a derivative of CoQ10, an analog of CoQ10, a metabolite of CoQ10, or an intermediate of the coenzyme biosynthesis pathway). One skilled in the art would be able, by routine experimentation, to determine what an effective, non-toxic amount would be for the purpose of treating malignancies. For example, a therapeutically active amount of a CoQ10 molecule (e.g., CoQ10, a building block of CoQ10, a derivative of CoQ10, an analog of CoQ10, a metabolite of CoQ10, or an intermediate of the coenzyme biosynthesis pathway) may vary according to factors such as the disease stage (e.g., stage I versus stage 1V), age, sex, medical complications (e g, immunosuppressed conditions or diseases) and weight of the subject, and the ability of the CoQ10 molecule to elicit a desired response in the subject. The dosage regimen may be adjusted to provide the optimum therapeutic response. For example, several divided doses may be administered daily, or the dose may be proportionally reduced as indicated by the exigencies of the therapeutic situation.

In one embodiment, the Coenzyme Q10 molecule, e.g., CoQ10, is topically applied one or more times per 24 hours for six weeks or more.

In one embodiment, the Coenzyme Q10 molecule, e.g., CoQ10, is administered in the form of a CoQ10 cream at a dosage of between 0.5 and 10 milligrams of the CoQ10 cream per square centimeter of skin, wherein the CoQ10 cream comprises between 1 and 5% of Coenzyme Q10. In one embodiment, the CoQ10 cream comprises about 3% of Coenzyme Q10. In one embodiment, the Coenzyme Q10 is administered in the form of a CoQ10 cream at a dosage of between 3 and 5 milligrams of the CoQ10 cream per square centimeter of skin, wherein the CoQ10 cream comprises between 1 and 5% of Coenzyme Q10. In one embodiment, the CoQ10 cream comprises about 3% of Coenzyme Q10.

In certain embodiments of the above methods of treatment or prevention, the method serves to modulate one or more genes (or proteins) selected from the group consisting of ANGPTL3, CCL2, CDH5, CXCL1, CXCL3, PRMT3, HDAC2, Nitric Oxide Synthase bNOS, Acetyl phospho Histone H3 AL9 S10, MTA 2, Glutamic Acid Decarboxylase GAD65 67, KSR, HDAC4, BOB1 OBF1, a1Syntrophin, BAP1, Importina 57, α E-Catenin, Grb2, Bax, Proteasome 26S subunit 13 (Endophilin B 1), Actin-like 6A (Eukaryotic Initiation Factor 4A11), Nuclear Chloride Channel protein, Proteasome 26S subunit, Dismutase Cu/Zn Superoxide, Translin-associated factor X, Arsenite translocating ATPase (Spermine synthetase), ribosomal protein SA, dCTP pyrophosphatase 1, proteasome beta 3, proteasome beta 4, acid phosphatase 1, diazepam binding inhibitor, alpha 2-HS glycoprotein (Bos Taurus, cow), ribosomal protein P2 (RPLP2); histone H2A, microtubule associated protein, proteasome alpha 3, eukaryotic translation elongation factor 1 delta, lamin B1, SMT 3 suppressor of mif two 3 homolog 2, heat shock protein 27 kD, hnRNP C1/C2, eukaryotc translation elongation factor 1 beta 2, Similar to HSPC-300, DNA directed DNA polymerase epislon 3; (canopy 2 homolog), LAMA5, PXLDC1, p300 CBP, P53R2, Phosphatidylserine Receptor, Cytokeratin Peptide 17, Cytokeratin peptide 13, Neurofilament 160 200, Rab5, Filensin, P53R2, MDM2, MSH6, Heat Shock Factor 2, AFX, FLIPg d, JAB 1, Myosine, MEKK4, cRaf pSer621, FKHR FOXO1a, MDM2, Fas Ligand, P53R2, Myosin Regulatory Light Chain, hnRNP C1/C2, Ubiquilin 1 (Phosphatase 2A), hnRNP C1/C2, alpha 2-HS glycoprotein (Bos Taurus, cow), beta actin, hnRNP C1/C2, heat shock protein 70 kD, beta tubulin, ATP dependent helicase II, eukaryotc translation elongation factor 1 beta 2, ER lipid raft associated 2 isoform 1 (beta actin), signal sequence receptor 1 delta, Eukaryotic translation initiation factor 3, subunit 3 gamma, Bilverdin reductase A (Transaldolase 1), Keratin 1,10 (Parathymosin), GST omega 1, chain B Dopamine Quinone Conjugation to Dj-1, Proteasome Activator Reg (alpha), T-complex protein 1 isoform A, Chain A Tapasin ERP57 (Chaperonin containing TCP1), Ubiquitin activating enzyme E1; Alanyl-tRNA synthetase, Dynactin 1, Heat shock protein 60 kd, Beta Actin, Spermidine synthase (Beta Actin), Heat Shock protein 70 kd, retinoblastoma binding protein 4 isoform A, TAR DNA binding protein, eukaryotic translation elongation factor 1 beta 2, chaperonin containing TCP1, subunit 3, cytoplasmic dynein IC-2, Angiotensin-converting enzyme (ACE), Caspase 3, GARS, Matrix Metalloproteinase 6 (MMP-6), Neurolysin (NLN)-Catalytic Domain, and Neurolysin (NLN), ADRB, CEACAM1, DUSP4, FOXC2, FOXP3, GCGR, GPD1, HMOX1, IL4R, INPPL1, IRS2, VEGFA, putative c-myc-responsive isoform 1, PDK 1, Caspase 12, Phospholipase D1, P34 cdc2, P53 BP1, BTK, ASC2, BUBR1, ARTS, PCAF, Raf1, MSK1, SNAP25, APRIL, DAPK, RAIDD, HAT1, PSF, HDAC1, Rad17, Surviving, SLIPR, MAG13, Caspase 10, Crk2, Cdc 6, P21 WAF 1 Cip 1, ASPP 1, HDAC 4, Cyclin B1, CD 40, GAD 65, TAP, Par4 (prostate apoptosis response 4), MRP1, MDC1, Laminin2 a2, bCatenin, FXR2, AnnexinV, SMAC Diablo, MBNL1, DImethyl Histone h3, Growth factor independence 1, U2AF65, mTOR, E2F2, Kaiso, Glycogen Synthase Kinase 3, ATF2, HDRP MITR, Neurabin I, AP1, and Apaf1. In some embodiments, the methods of treatment or prevention serve to modulate a combination of at least two, three, four, five, six, seven, eight, nine, ten, eleven, twelve, thirteen, fourteen, fifteen, sixteen, seventeen, eighteen, nineteen, twenty, twenty-five, thirty or more of the foregoing genes (or proteins).

In some embodiments, the methods of treatment or prevention of the invention serve to upregulate the level of expression of one or more genes or any combinations of genes selected from the group consisting of LAMA5, PXLDC1, p300 CBP, P53R2, Phosphatidylserine Receptor, Cytokeratin Peptide 17, Cytokeratin peptide 13, Neurofilament 160 200, Rab5, Filensin, P53R2, MDM2, MSH6, Heat Shock Factor 2, AFX, FLIPg d, JAB 1, Myosine, MEKK4, cRaf pSer621, FKHR FOXO1a, MDM2, Fas Ligand, P53R2, Proteasome 26S subunit 13 (Endophilin B 1), Myosin Regulatory Light Chain, hnRNP C1/C2, Ubiquilin 1 (Phosphatase 2A), hnRNP C1/C2, alpha 2-HS glycoprotein (Bos Taurus, cow), beta actin, hnRNP C1/C2, heat shock protein 70 kD, microtubule associated protein, beta tubulin, proteasome alpha 3, ATP dependent helicase II, eukaryotic translation elongation factor 1 delta, heat shock protein 27 kD, eukaryotc translation elongation factor 1 beta 2, Similar to HSPC-300, ER lipid raft associated 2 isoform 1 (beta actin), Dismutase Cu/Zn Superoxide, and signal sequence receptor 1 delta, ADRB, CEACAM1, DUSP4, FOXC2, FOXP3, GCGR, GPD1, HMOX1, IL4R, INPPL1, IRS2 and VEGFA, putative c-myc-responsive isoform 1, PDK 1, Caspase 12, Phospholipase D1, P34 cdc2, P53 BP1, BTK, ASC2, BUBR1, ARTS, PCAF, Raf1, MSK1, SNAP25, APRIL, DAPK, RAIDD, HAT1, PSF, HDAC1, Rad17, Surviving, SLIPR, MAG13, Caspase 10, Crk2, Cdc 6, P21 WAF 1 Cip 1, ASPP 1, HDAC 4, Cyclin B1, CD 40, GAD 65, TAP, Par4 (prostate apoptosis response 4), and MRP1. In some embodiments, the methods of treatment or prevention serve to upregulate a combination of at least two, three, four, five, six, seven, eight, nine, ten, eleven, twelve, thirteen, fourteen, fifteen, sixteen, seventeen, eighteen, nineteen, twenty, twenty-five, thirty or more of the foregoing genes (or proteins).

In further embodiments, the methods of treatment or prevention provided by the invention serve to downregulate the level of expression of one or more genes or any combinations of genes selected from the group consisting of ANGPTL3, CCL2, CDH5, CXCL1, CXCL3, PRMT3, HDAC2, Nitric Oxide Synthase bNOS, Acetyl phospho Histone H3 AL9 S10, MTA 2, Glutamic Acid Decarboxylase GAD65 67, KSR, HDAC4, BOB1 OBF1, a1Syntrophin, BAP1, Importina 57, α E-Catenin, Grb2, Bax, Proteasome 26S subunit 13 (Endophilin B 1), Actin-like 6A (Eukaryotic Initiation Factor 4A11), Nuclear Chloride Channel protein, Proteasome 26S subunit, Dismutase Cu/Zn Superoxide, Translin-associated factor X, Arsenite translocating ATPase (Spermine synthetase), ribosomal protein SA, dCTP pyrophosphatase 1, proteasome beta 3, proteasome beta 4, acid phosphatase 1, diazepam binding inhibitor, ribosomal protein P2 (RPLP2); histone H2A, microtubule associated protein, proteasome alpha 3, eukaryotic translation elongation factor 1 delta, lamin B1, SMT 3 suppressor of mif two 3 homolog 2, heat shock protein 27 kD, hnRNP C1/C2, eukaryotc translation elongation factor 1 beta 2, Similar to HSPC-300, DNA directed DNA polymerase epislon 3 (canopy 2 homolog), Angiotensin-converting enzyme (ACE), Caspase 3, GARS, Matrix Metalloproteinase 6 (MMP-6), Neurolysin (NLN)-Catalytic Domain, Neurolysin (NLN), MDC1, Laminin2 a2, bCatenin, FXR2, AnnexinV, SMAC Diablo, MBNL1, DImethyl Histone h3, Growth factor independence 1, U2AF65, mTOR, E2F2, Kaiso, Glycogen Synthase Kinase 3, ATF2, HDRP MITR, Neurabin I, AP1, and Apaf1. In some embodiments, the methods of treatment or prevention serve to downregulate a combination of at least two, three, four, five, six, seven, eight, nine, ten, eleven, twelve, thirteen, fourteen, fifteen, sixteen, seventeen, eighteen, nineteen, twenty, twenty-five, thirty or more of the foregoing genes (or proteins).

In one embodiment, the methods of treatment or prevention provided by the present invention serve to modulate the level of expression of genes involved in diabetes. Such genes may include, for example, ADRB, CEACAM1, DUSP4, FOX C2, FOXP3, GCGR, GPD1, HMOX1, IL4R, INPPL1, IRS2, VEGFA, ANGPTL3, CCL2, CDH5, CXCL1, CXCL3, LAMA5, and/or PXLDC1. In some embodiments, the methods of treatment or prevention serve to modulate a combination of at least two, three, four, five, six, seven, eight, nine, ten, eleven, twelve, thirteen, fourteen, fifteen, sixteen, seventeen, eighteen, or all nineteen, of the foregoing genes (or proteins).

In a further embodiment, the methods of treatment or prevention serve to upregulate the level of expression of genes involved in diabetes. Such genes may include, for example, ADRB, CEACAM1, DUSP4, FOX C2, FOXP3, GCGR, GPD1, HMOX1, IL4R, INPPL1, IRS2, and/or VEGFA. In some embodiments, the methods of treatment or prevention upregulate a combination of at least two, three, four, five, six, seven, eight, nine, ten, eleven, or all twelve of the foregoing genes (or proteins).

In a further embodiment, the method of treatment or prevention serves to downregulate the level of expression of genes involved in diabetes. Such genes may include, for example, ANGPTL3, CCL2, CDH5, CXCL1, CXCL3, LAMA5, and/or PXLDC1. In some embodiments, the methods of treatment or prevention downregulate a combination of at least two, three, four, five, six, or all seven of the foregoing genes (or proteins).

In yet another embodiment, the method of treatment or prevention serves to modulate the level of expression of genes involved in angiogenesis. Such genes may include, for example, ANGPTL3, CCL2, CDH5, CXCL1, CXCL3, LAMA5, and/or PXLDC1. In some embodiments, the methods of treatment or prevention modulate a combination of at least two, three, four, five, six, or all seven genes from the foregoing group.

In a further embodiment, the method of treatment or prevention serves to upregulate the level of expression of genes involved in angiogenesis. Such genes may include, for example, ANGPTL3, CCL2, CDH5, CXCL1, and/or CXCL3. In some embodiments, the methods of treatment or prevention upregulate a combination of at least two, three, four, or all five, genes from the foregoing group.

In a further embodiment, the methods of treatment or prevention serve to downregulate the level of expression of genes involved in angiogenesis. Such genes may include, for example, LAMA5, and/or PXLDC1. In one embodiment, the methods of treatment or prevention downregulate both LAMA5 and PXLDC1.

In another embodiment, the methods of treatment or prevention serve to modulate the level of expression of genes involved in apoptosis. Such genes may include, for example, genes that were modulated in the experiments described herein, i.e., the genes listed in Tables 2-9. In another embodiment, the genes or proteins involved in apoptosis include one or more of JAB1, p53R2, phosphatidylserine receptor, Rab 5, AFX, MEKK4, HDAC2, HDAC4, PDK1, Caspase12, phospholipase D1, p34cdc2, BTK, ASC2, BubR1, PCAF, Raf1, MSK1, and mTOR. In some embodiments, the methods of treatment or prevention modulate a combination of at least two, three, four, five, six, seven, eight, nine, ten, eleven, twelve, thirteen, fourteen, fifteen, sixteen, seventeen, eighteen, or all nineteen genes from the foregoing group.

### V. Diagnostic Methods of the Invention

The invention provides methods for diagnosing a sarcoma. The methods of the present invention can be practiced in conjunction with any other method used by the skilled practitioner to prognose the recurrence of a sarcoma and/or the survival of a subject being treated for a sarcoma. For example, the methods of the invention may be performed in conjunction with a morphological or cytological analysis of the sample obtained from the subject. Cytological methods would include immunohistochemical or immunofluorescence detection (and quantitation if appropriate) of any other molecular marker either by itself, in conjunction with other markers, and/or in conjunction with the Shc markers. Other methods would include detection of other markers by in situ PCR, or by extracting tissue and quantitating other markers by real time PCR. PCR is defined as polymerase chain reaction.

Methods for assessing the efficacy of a treatment regimen, e.g., chemotherapy, radiation therapy, surgery, hormone therapy, or any other therapeutic approach useful for treating an oncologic disorder in a subject are also provided. In these methods the amount of marker in a pair of samples (a first sample not subjected to the treatment regimen and a second sample subjected to at least a portion of the treatment regimen) is assessed.

The invention also provides a method for determining whether a sarcoma is aggressive. The method comprises determining the amount of marker present in a cell and comparing the amount to a control amount of marker present in a control sample, defined in Definitions, thereby determining whether a sarcoma is aggressive.

The methods of the invention may also be used to select a compound that is capable of modulating, i.e., decreasing, the aggressiveness of a sarcoma. In this method, a cancer cell is contacted with a test compound, and the ability of the test compound to modulate the expression and/or activity of a marker of the invention in the sarcoma cell is determined, thereby selecting a compound that is capable of modulating aggressiveness of the sarcoma.

Using the methods described herein, a variety of molecules, particularly including molecules sufficiently small to be able to cross the cell membrane, may be screened in order to identify molecules which modulate, e.g., increase the expression and/or activity of a marker of the invention. Compounds so identified can be provided to a subject in order to inhibit the aggressiveness of a sarcoma in the subject, to prevent the recurrence of a sarcoma in the subject, or to treat a sarcoma in the subject.

### VI. Markers of the Invention

The invention relates to markers (hereinafter “markers” or “markers of the invention”), which are listed in Tables 2-9. The invention provides nucleic acids and proteins that are encoded by or correspond to the markers (hereinafter “marker nucleic acids” and “marker proteins,” respectively). These markers are particularly useful in screening for the presence of a sarcoma, in assessing aggressiveness and metastatic potential of a sarcoma, assessing whether a subject is afflicted with aa sarcoma, identifying a composition for treating a sarcoma, assessing the efficacy of an environmental influencer compound for treating a sarcoma, monitoring the progression of a sarcoma, prognosing the aggressiveness of a sarcoma, prognosing the survival of a subject with a sarcoma, prognosing the recurrence of a sarcoma and prognosing whether a subject is predisposed to developing a sarcoma.

A “marker” is a gene whose altered level of expression in a tissue or cell from its expression level in normal or healthy tissue or cell is associated with a disease state, such as a sarcoma. A “marker nucleic acid” is a nucleic acid (e.g., mRNA, cDNA) encoded by or corresponding to a marker of the invention. Such marker nucleic acids include DNA (e.g., cDNA) comprising the entire or a partial sequence of any of the genes that are markers of the invention or the complement of such a sequence. Such sequences are known to the one of skill in the art and can be found for example, on the NIH government pubmed website. The marker nucleic acids also include RNA comprising the entire or a partial sequence of any of the gene markers of the invention or the complement of such a sequence, wherein all thymidine residues are replaced with uridine residues. A “marker protein” is a protein encoded by or corresponding to a marker of the invention. A marker protein comprises the entire or a partial sequence of any of the marker proteins of the invention. Such sequences are known to the one of skill in the art and can be found for example, on the NIH government pubmed website. The terms “protein” and “polypeptide’ are used interchangeably.

An “sarcoma-associated” body fluid is a fluid which, when in the body of a patient, contacts or passes through sarcoma cells or into which cells or proteins shed from sarcoma cells are capable of passing. Exemplary sarcoma-associated body fluids include blood fluids (e.g. whole blood, blood serum, blood having platelets removed therefrom), and are described in more detail below. Many sarcoma disorder-associated body fluids can have sarcoma cells therein, particularly when the cells are metastasizing. Cell-containing fluids which can contain sarcoma cells include, but are not limited to, whole blood, blood having platelets removed therefrom, lymph, prostatic fluid, urine and semen.

The “normal” level of expression of a marker is the level of expression of the marker in cells of a human subject or patient not afflicted with sarcoma.

An “over-expression” or “higher level of expression” of a marker refers to an expression level in a test sample that is greater than the standard error of the assay employed to assess expression, and is preferably at least twice, and more preferably three, four, five, six, seven, eight, nine or ten times the expression level of the marker in a control sample (e.g., sample from a healthy subject not having the marker associated disease, i.e., sarcoma) and preferably, the average expression level of the marker in several control samples.

A “lower level of expression” of a marker refers to an expression level in a test sample that is at least twice, and more preferably three, four, five, six, seven, eight, nine or ten times lower than the expression level of the marker in a control sample (e.g., sample from a healthy subjects not having the marker associated disease, i.e., sarcoma) and preferably, the average expression level of the marker in several control samples.

A “transcribed polynucleotide” or “nucleotide transcript” is a polynucleotide (e.g. an mRNA, hnRNA, a cDNA, or an analog of such RNA or cDNA) which is complementary to or homologous with all or a portion of a mature mRNA made by transcription of a marker of the invention and normal post-transcriptional processing (e.g. splicing), if any, of the RNA transcript, and reverse transcription of the RNA transcript.

“Complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

“Homologous” as used herein, refers to nucleotide sequence similarity between two regions of the same nucleic acid strand or between regions of two different nucleic acid strands. When a nucleotide residue position in both regions is occupied by the same nucleotide residue, then the regions are homologous at that position. A first region is homologous to a second region if at least one nucleotide residue position of each region is occupied by the same residue. Homology between two regions is expressed in terms of the proportion of nucleotide residue positions of the two regions that are occupied by the same nucleotide residue. By way of example, a region having the nucleotide sequence 5′-ATTGCC-3′ and a region having the nucleotide sequence 5′-TATGGC-3′ share 50% homology. Preferably, the first region comprises a first portion and the second region comprises a second portion, whereby, at least about 50%, and preferably at least about 75%, at least about 90%, or at least about 95% of the nucleotide residue positions of each of the portions are occupied by the same nucleotide residue. More preferably, all nucleotide residue positions of each of the portions are occupied by the same nucleotide residue.

“Proteins of the invention” encompass marker proteins and their fragments; variant marker proteins and their fragments; peptides and polypeptides comprising an at least 15 amino acid segment of a marker or variant marker protein; and fusion proteins comprising a marker or variant marker protein, or an at least 15 amino acid segment of a marker or variant marker protein.

The invention further provides antibodies, antibody derivatives and antibody fragments which specifically bind with the marker proteins and fragments of the marker proteins of the present invention. Unless otherwise specified herewithin, the terms “antibody” and “antibodies” broadly encompass naturally-occurring forms of antibodies (e.g., IgG, IgA, IgM, IgE) and recombinant antibodies such as single-chain antibodies, chimeric and humanized antibodies and multi-specific antibodies, as well as fragments and derivatives of all of the foregoing, which fragments and derivatives have at least an antigenic binding site. Antibody derivatives may comprise a protein or chemical moiety conjugated to an antibody.

In certain embodiments, the markers of the invention include one or more genes (or proteins) selected from the group consisting of ANGPTL3, CCL2, CDH5, CXCL1, CXCL3, PRMT3, HDAC2, Nitric Oxide Synthase bNOS, Acetyl phospho Histone H3 AL9 S10, MTA 2, Glutamic Acid Decarboxylase GAD65 67, KSR, HDAC4, BOB1 OBF1, a1Syntrophin, BAP1, Importina 57, α E-Catenin, Grb2, Bax, Proteasome 26S subunit 13 (Endophilin B 1), Actin-like 6A (Eukaryotic Initiation Factor 4A11), Nuclear Chloride Channel protein, Proteasome 26S subunit, Dismutase Cu/Zn Superoxide, Translin-associated factor X, Arsenite translocating ATPase (Spermine synthetase), ribosomal protein SA, dCTP pyrophosphatase 1, proteasome beta 3, proteasome beta 4, acid phosphatase 1, diazepam binding inhibitor, alpha 2-HS glycoprotein (Bos Taurus, cow), ribosomal protein P2 (RPLP2); histone H2A, microtubule associated protein, proteasome alpha 3, eukaryotic translation elongation factor 1 delta, lamin B1, SMT 3 suppressor of mif two 3 homolog 2, heat shock protein 27 kD, hnRNP C1/C2, eukaryotc translation elongation factor 1 beta 2, Similar to HSPC-300, DNA directed DNA polymerase epislon 3; (canopy 2 homolog), LAMA5, PXLDC1, p300 CBP, P53R2, Phosphatidylserine Receptor, Cytokeratin Peptide 17, Cytokeratin peptide 13, Neurofilament 160 200, Rab5, Filensin, P53R2, MDM2, MSH6, Heat Shock Factor 2, AFX, FLIPg d, JAB 1, Myosine, MEKK4, cRaf pSer621, FKHR FOXO1a, MDM2, Fas Ligand, P53R2, Myosin Regulatory Light Chain, hnRNP C1/C2, Ubiquilin 1 (Phosphatase 2A), hnRNP C1/C2, alpha 2-HS glycoprotein (Bos Taurus, cow), beta actin, hnRNP C1/C2, heat shock protein 70 kD, beta tubulin, ATP dependent helicase II, eukaryotc translation elongation factor 1 beta 2, ER lipid raft associated 2 isoform 1 (beta actin), signal sequence receptor 1 delta, Eukaryotic translation initiation factor 3, subunit 3 gamma, Bilverdin reductase A (Transaldolase 1), Keratin 1,10 (Parathymosin), GST omega 1, chain B Dopamine Quinone Conjugation to Dj-1, Proteasome Activator Reg (alpha), T-complex protein 1 isoform A, Chain A Tapasin ERP57 (Chaperonin containing TCP1), Ubiquitin activating enzyme E1; Alanyl-tRNA synthetase, Dynactin 1, Heat shock protein 60 kd, Beta Actin, Spermidine synthase (Beta Actin), Heat Shock protein 70 kd, retinoblastoma binding protein 4 isoform A, TAR DNA binding protein, eukaryotic translation elongation factor 1 beta 2, chaperonin containing TCP1, subunit 3, cytoplasmic dynein IC-2, Angiotensin-converting enzyme (ACE), Caspase 3, GARS, Matrix Metalloproteinase 6 (MMP-6), Neurolysin (NLN)-Catalytic Domain, and Neurolysin (NLN), ADRB, CEACAM1, DUSP4, FOXC2, FOXP3, GCGR, GPD1, HMOX1, IL4R, INPPL1, IRS2, VEGFA, putative c-myc-responsive isoform 1, PDK 1, Caspase 12, Phospholipase D1, P34 cdc2, P53 BP1, BTK, ASC2, BUBR1, ARTS, PCAF, Raf1, MSK1, SNAP25, APRIL, DAPK, RAIDD, HAT1, PSF, HDAC1, Rad17, Surviving, SLIPR, MAG13, Caspase 10, Crk2, Cdc 6, P21 WAF 1 Cip 1, ASPP 1, HDAC 4, Cyclin B1, CD 40, GAD 65, TAP, Par4 (prostate apoptosis response 4), MRP1, MDC1, Laminin2 a2, bCatenin, FXR2, AnnexinV, SMAC Diablo, MBNL1, DImethyl Histone h3, Growth factor independence 1, U2AF65, mTOR, E2F2, Kaiso, Glycogen Synthase Kinase 3, ATF2, HDRP MITR, Neurabin I, AP1, and Apaf1. In some embodiments, the markers are a combination of at least two, three, four, five, six, seven, eight, nine, ten, eleven, twelve, thirteen, fourteen, fifteen, sixteen, seventeen, eighteen, nineteen, twenty, twenty-five, thirty, thirty-five, forty, forty-five, fifty or more of the foregoing genes (or proteins).

In some embodiments, the markers of the invention are genes or proteins that are upregulated upon treatment of a sarcoma cell with Coenzyme Q10. Markers that are upregulated upon treatment of a sarcoma with Coenzyme Q10 include LAMA5, PXLDC1, p300 CBP, P53R2, Phosphatidylserine Receptor, Cytokeratin Peptide 17, Cytokeratin peptide 13, Neurofilament 160 200, Rab5, Filensin, P53R2, MDM2, MSH6, Heat Shock Factor 2, AFX, FLIPg d, JAB 1, Myosine, MEKK4, cRaf pSer621, FKHR FOXO1a, MDM2, Fas Ligand, P53R2, Proteasome 26S subunit 13 (Endophilin B1), Myosin Regulatory Light Chain, hnRNP C1/C2, Ubiquilin 1 (Phosphatase 2A), hnRNP C1/C2, alpha 2-HS glycoprotein (Bos Taurus, cow), beta actin, hnRNP C1/C2, heat shock protein 70 kD, microtubule associated protein, beta tubulin, proteasome alpha 3, ATP dependent helicase II, eukaryotic translation elongation factor 1 delta, heat shock protein 27 kD, eukaryotc translation elongation factor 1 beta 2, Similar to HSPC-300, ER lipid raft associated 2 isoform 1 (beta actin), Dismutase Cu/Zn Superoxide, and signal sequence receptor 1 delta, ADRB, CEACAM1, DUSP4, FOXC2, FOXP3, GCGR, GPD1, HMOX1, IL4R, INPPL1, IRS2 and VEGFA, putative c-myc-responsive isoform 1, PDK 1, Caspase 12, Phospholipase D1, P34 cdc2, P53 BP1, BTK, ASC2, BUBR1, ARTS, PCAF, Raf1, MSK1, SNAP25, APRIL, DAPK, RAIDD, HAT1, PSF, HDAC1, Rad17, Surviving, SLIPR, MAG13, Caspase 10, Crk2, Cdc 6, P21 WAF 1 Cip 1, ASPP 1, HDAC 4, Cyclin B1, CD 40, GAD 65, TAP, Par4 (prostate apoptosis response 4), and MRP1. In some embodiments, the upregulated markers are a combination of at least two, three, four, five, six, seven, eight, nine, ten, eleven, twelve, thirteen, fourteen, fifteen, sixteen, seventeen, eighteen, nineteen, twenty, twenty-five, thirty or more of the foregoing genes (or proteins).

In further embodiments, the markers are genes or proteins that are down-regulated in a sarcoma cell upon treatment with CoQ10. Markers that are downregulated include ANGPTL3, CCL2, CDH5, CXCL1, CXCL3, PRMT3, HDAC2, Nitric Oxide Synthase bNOS, Acetyl phospho Histone H3 AL9 S10, MTA 2, Glutamic Acid Decarboxylase GAD65 67, KSR, HDAC4, BOB1 OBF1, a1Syntrophin, BAP1, Importina 57, α E-Catenin, Grb2, Bax, Proteasome 26S subunit 13 (Endophilin B1), Actin-like 6A (Eukaryotic Initiation Factor 4A11), Nuclear Chloride Channel protein, Proteasome 26S subunit, Dismutase Cu/Zn Superoxide, Translin-associated factor X, Arsenite translocating ATPase (Spermine synthetase), ribosomal protein SA, dCTP pyrophosphatase 1, proteasome beta 3, proteasome beta 4, acid phosphatase 1, diazepam binding inhibitor, ribosomal protein P2 (RPLP2); histone H2A, microtubule associated protein, proteasome alpha 3, eukaryotic translation elongation factor 1 delta, lamin B 1, SMT 3 suppressor of mif two 3 homolog 2, heat shock protein 27 kD, hnRNP C1/C2, eukaryotc translation elongation factor 1 beta 2, Similar to HSPC-300, DNA directed DNA polymerase epislon 3 (canopy 2 homolog), Angiotensin-converting enzyme (ACE), Caspase 3, GARS, Matrix Metalloproteinase 6 (MMP-6), Neurolysin (NLN)-Catalytic Domain, Neurolysin (NLN), MDC1, Laminin2 a2, bCatenin, FXR2, AnnexinV, SMAC Diablo, MBNL1, DImethyl Histone h3, Growth factor independence 1, U2AF65, mTOR, E2F2, Kaiso, Glycogen Synthase Kinase 3, ATF2, HDRP MITR, Neurabin I, AP1, and Apaf1. In some embodiments, the downregulated markers are a combination of at least two, three, four, five, six, seven, eight, nine, ten, eleven, twelve, thirteen, fourteen, fifteen, sixteen, seventeen, eighteen, nineteen, twenty, twenty-five, thirty or more of the foregoing genes (or proteins).

In one embodiment, the markers of the invention are genes or proteins associated with or involved in diabetes. Such genes or proteins involved in diabetes include, for example, ADRB, CEACAM1, DUSP4, FOX C2, FOXP3, GCGR, GPD1, HMOX1, IL4R, INPPL1, IRS2, VEGFA, ANGPTL3, CCL2, CDH5, CXCL1, CXCL3, LAMA5, and/or PXLDC1. In some embodiments, the markers of the invention are a combination of at least two, three, four, five, six, seven, eight, nine, ten, eleven, twelve, thirteen, fourteen, fifteen, sixteen, seventeen, eighteen, or all nineteen, of the foregoing genes (or proteins).

In one embodiment, the markers associated with or involved in diabetes are genes or proteins that are upregulated upon treatment of a sarcoma cell with CoQ10. Such markers include, for example, ADRB, CEACAM1, DUSP4, FOX C2, FOXP3, GCGR, GPD1, HMOX1, IL4R, INPPL1, IRS2, and/or VEGFA. In some embodiments, the upregulated markers involved in diabetes are a combination of at least two, three, four, five, six, seven, eight, nine, ten, eleven, or all twelve of the foregoing genes (or proteins).

In a further embodiment, the markers associated with or involved in diabetes are genes or proteins that are downregulated upon treatment of a sarcoma cell with CoQ10. Such genes include, for example, ANGPTL3, CCL2, CDH5, CXCL1, CXCL3, LAMA5, and/or PXLDC1. In some embodiments, the downregulated markers involved in diabetes are a combination of at least two, three, four, five, six, or all seven of the foregoing genes (or proteins).

In yet another embodiment, the markers of the invention are genes or proteins associated with or involved in angiogenesis. Such genes may include, for example, ANGPTL3, CCL2, CDH5, CXCL1, CXCL3, LAMA5, and/or PXLDC1. In some embodiments, the markers involved in angiogenesis are a combination of at least two, three, four, five, six, or all seven genes from the foregoing group.

In a further embodiment, the markers associated with or involved in angiogenesis are genes or proteins that are upregulated upon treatment of a sarcoma cell with CoQ10. Such genes may include, for example, ANGPTL3, CCL2, CDH5, CXCL1, and/or CXCL3. In some embodiments, the upregulate markers associated with angiogenesis are a combination of at least two, three, four, or all five, genes from the foregoing group.

In a further embodiment, the markers associated with or involved in angiogenesis are genes or proteins that are downregulated upon treatment of a sarcoma cell with CoQ10. Such genes may include, for example, LAMA5, and/or PXLDC1. In one embodiment, the downregulate markers are both LAMA5 and PXLDC1.

In another embodiment, the markers are genes or proteins involved in apoptosis. Such genes may include, for example, the genes listed in Tables 2-9. In one embodiment, the markers involved in apoptosis include JAB1, p53R2, phosphatidylserine receptor, Rab 5, AFX, MEKK4, HDAC2, HDAC4, PDK1, Caspase12, phospholipase D1, p34cdc2, BTK, ASC2, BubR1, PCAF, Raf1, MSK1, and mTOR.

Various aspects of the invention are described in further detail in the following subsections.

### 1. Isolated Nucleic Acid Molecules

One aspect of the invention pertains to isolated nucleic acid molecules, including nucleic acids which encode a marker protein or a portion thereof. Isolated nucleic acids of the invention also include nucleic acid molecules sufficient for use as hybridization probes to identify marker nucleic acid molecules, and fragments of marker nucleic acid molecules, e.g., those suitable for use as PCR primers for the amplification or mutation of marker nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

An “isolated” nucleic acid molecule is one which is separated from other nucleic acid molecules which are present in the natural source of the nucleic acid molecule. In one embodiment, an “isolated” nucleic acid molecule is free of sequences (preferably protein-encoding sequences) which naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kB, 4 kB, 3 kB, 2 kB, 1 kB, 0.5 kB or 0.1 kB of nucleotide sequences which naturally flank the nucleic acid molecule in genomic DNA of the cell from which the nucleic acid is derived. In another embodiment, an “isolated” nucleic acid molecule, such as a cDNA molecule, can be substantially free of other cellular material, or culture medium when produced by recombinant techniques, or substantially free of chemical precursors or other chemicals when chemically synthesized. A nucleic acid molecule that is substantially free of cellular material includes preparations having less than about 30%, 20%, 10%, or 5% of heterologous nucleic acid (also referred to herein as a “contaminating nucleic acid”).

A nucleic acid molecule of the present invention can be isolated using standard molecular biology techniques and the sequence information in the database records described herein. Using all or a portion of such nucleic acid sequences, nucleic acid molecules of the invention can be isolated using standard hybridization and cloning techniques (e.g., as described in Sambrook et al., ed., Molecular Cloning: A Laboratory Manual, 2nd ed., Cold Spring Harbor Laboratory Press, Cold Spring Harbor, N.Y., 1989).

A nucleic acid molecule of the invention can be amplified using cDNA, mRNA, or genomic DNA as a template and appropriate oligonucleotide primers according to standard PCR amplification techniques. The nucleic acid so amplified can be cloned into an appropriate vector and characterized by DNA sequence analysis. Furthermore, nucleotides corresponding to all or a portion of a nucleic acid molecule of the invention can be prepared by standard synthetic techniques, e.g., using an automated DNA synthesizer.

In another preferred embodiment, an isolated nucleic acid molecule of the invention comprises a nucleic acid molecule which has a nucleotide sequence complementary to the nucleotide sequence of a marker nucleic acid or to the nucleotide sequence of a nucleic acid encoding a marker protein. A nucleic acid molecule which is complementary to a given nucleotide sequence is one which is sufficiently complementary to the given nucleotide sequence that it can hybridize to the given nucleotide sequence thereby forming a stable duplex.

Moreover, a nucleic acid molecule of the invention can comprise only a portion of a nucleic acid sequence, wherein the full length nucleic acid sequence comprises a marker nucleic acid or which encodes a marker protein. Such nucleic acids can be used, for example, as a probe or primer. The probe/primer typically is used as one or more substantially purified oligonucleotides. The oligonucleotide typically comprises a region of nucleotide sequence that hybridizes under stringent conditions to at least about 7, preferably about 15, more preferably about 25, 50, 75, 100, 125, 150, 175, 200, 250, 300, 350, or 400 or more consecutive nucleotides of a nucleic acid of the invention.

Probes based on the sequence of a nucleic acid molecule of the invention can be used to detect transcripts or genomic sequences corresponding to one or more markers of the invention. The probe comprises a label group attached thereto, e.g., a radioisotope, a fluorescent compound, an enzyme, or an enzyme co-factor. Such probes can be used as part of a diagnostic test kit for identifying cells or tissues which mis-express the protein, such as by measuring levels of a nucleic acid molecule encoding the protein in a sample of cells from a subject, e.g., detecting mRNA levels or determining whether a gene encoding the protein has been mutated or deleted.

The invention further encompasses nucleic acid molecules that differ, due to degeneracy of the genetic code, from the nucleotide sequence of nucleic acids encoding a marker protein, and thus encode the same protein.

It will be appreciated by those skilled in the art that DNA sequence polymorphisms that lead to changes in the amino acid sequence can exist within a population (e.g., the human population). Such genetic polymorphisms can exist among individuals within a population due to natural allelic variation. An allele is one of a group of genes which occur alternatively at a given genetic locus. In addition, it will be appreciated that DNA polymorphisms that affect RNA expression levels can also exist that may affect the overall expression level of that gene (e.g., by affecting regulation or degradation).

As used herein, the phrase “allelic variant” refers to a nucleotide sequence which occurs at a given locus or to a polypeptide encoded by the nucleotide sequence.

As used herein, the terms “gene” and “recombinant gene” refer to nucleic acid molecules comprising an open reading frame encoding a polypeptide corresponding to a marker of the invention. Such natural allelic variations can typically result in 1-5% variance in the nucleotide sequence of a given gene. Alternative alleles can be identified by sequencing the gene of interest in a number of different individuals. This can be readily carried out by using hybridization probes to identify the same genetic locus in a variety of individuals. Any and all such nucleotide variations and resulting amino acid polymorphisms or variations that are the result of natural allelic variation and that do not alter the functional activity are intended to be within the scope of the invention.

In another embodiment, an isolated nucleic acid molecule of the invention is at least 7, 15, 20, 25, 30, 40, 60, 80, 100, 150, 200, 250, 300, 350, 400, 450, 550, 650, 700, 800, 900, 1000, 1200, 1400, 1600, 1800, 2000, 2200, 2400, 2600, 2800, 3000, 3500, 4000, 4500, or more nucleotides in length and hybridizes under stringent conditions to a marker nucleic acid or to a nucleic acid encoding a marker protein. As used herein, the term “hybridizes under stringent conditions” is intended to describe conditions for hybridization and washing under which nucleotide sequences at least 60% (65%, 70%, preferably 75%) identical to each other typically remain hybridized to each other. Such stringent conditions are known to those skilled in the art and can be found in sections 6.3.1-6.3.6 of Current Protocols in Molecular Biology, John Wiley & Sons, N.Y. (1989). A preferred, non-limiting example of stringent hybridization conditions are hybridization in 6× sodium chloride/sodium citrate (SSC) at about 45° C., followed by one or more washes in 0.2×SSC, 0.1% SDS at 50-65° C.

In addition to naturally-occurring allelic variants of a nucleic acid molecule of the invention that can exist in the population, the skilled artisan will further appreciate that sequence changes can be introduced by mutation thereby leading to changes in the amino acid sequence of the encoded protein, without altering the biological activity of the protein encoded thereby. For example, one can make nucleotide substitutions leading to amino acid substitutions at “non-essential” amino acid residues. A “non-essential” amino acid residue is a residue that can be altered from the wild-type sequence without altering the biological activity, whereas an “essential” amino acid residue is required for biological activity. For example, amino acid residues that are not conserved or only semi-conserved among homologs of various species may be non-essential for activity and thus would be likely targets for alteration. Alternatively, amino acid residues that are conserved among the homologs of various species (e.g., murine and human) may be essential for activity and thus would not be likely targets for alteration.

Accordingly, another aspect of the invention pertains to nucleic acid molecules encoding a variant marker protein that contain changes in amino acid residues that are not essential for activity. Such variant marker proteins differ in amino acid sequence from the naturally-occurring marker proteins, yet retain biological activity. In one embodiment, such a variant marker protein has an amino acid sequence that is at least about 40% identical, 50%, 60%, 70%, 80%, 90%, 91%, 92%, 93%, 94%, 95%, 96%, 97%, 98% or 99% identical to the amino acid sequence of a marker protein.

An isolated nucleic acid molecule encoding a variant marker protein can be created by introducing one or more nucleotide substitutions, additions or deletions into the nucleotide sequence of marker nucleic acids, such that one or more amino acid residue substitutions, additions, or deletions are introduced into the encoded protein. Mutations can be introduced by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed recombinantly and the activity of the protein can be determined.

The present invention encompasses antisense nucleic acid molecules, i.e., molecules which are complementary to a sense nucleic acid of the invention, e.g., complementary to the coding strand of a double-stranded marker cDNA molecule or complementary to a marker mRNA sequence. Accordingly, an antisense nucleic acid of the invention can hydrogen bond to (i.e. anneal with) a sense nucleic acid of the invention. The antisense nucleic acid can be complementary to an entire coding strand, or to only a portion thereof, e.g., all or part of the protein coding region (or open reading frame). An antisense nucleic acid molecule can also be antisense to all or part of a non-coding region of the coding strand of a nucleotide sequence encoding a marker protein. The non-coding regions (“5′ and 3′ untranslated regions”) are the 5′ and 3′ sequences which flank the coding region and are not translated into amino acids.

An antisense oligonucleotide can be, for example, about 5, 10, 15, 20, 25, 30, 35, 40, 45, or 50 or more nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N2-carboxypropyl) uracil, (acp3)w, and 2,6-diaminopurine. Alternatively, the antisense nucleic acid can be produced biologically using an expression vector into which a nucleic acid has been sub-cloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a marker protein to thereby inhibit expression of the marker, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. Examples of a route of administration of antisense nucleic acid molecules of the invention includes direct injection at a tissue site or infusion of the antisense nucleic acid into sarcoma-associated body fluid. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

An antisense nucleic acid molecule of the invention can be an α-anomeric nucleic acid molecule. An α-anomeric nucleic acid molecule forms specific double-stranded hybrids with complementary RNA in which, contrary to the usual α-units, the strands run parallel to each other (Gaultier et al., 1987, Nucleic Acids Res. 15:6625-6641). The antisense nucleic acid molecule can also comprise a 2′-o-methylribonucleotide (Inoue et al., 1987, Nucleic Acids Res. 15:6131-6148) or a chimeric RNA-DNA analogue (Inoue et al., 1987, FEBS Lett. 215:327-330).

The invention also encompasses ribozymes. Ribozymes are catalytic RNA molecules with ribonuclease activity which are capable of cleaving a single-stranded nucleic acid, such as an mRNA, to which they have a complementary region. Thus, ribozymes (e g, hammerhead ribozymes as described in Haselhoff and Gerlach, 1988, Nature 334:585-591) can be used to catalytically cleave mRNA transcripts to thereby inhibit translation of the protein encoded by the mRNA. A ribozyme having specificity for a nucleic acid molecule encoding a marker protein can be designed based upon the nucleotide sequence of a cDNA corresponding to the marker. For example, a derivative of a Tetrahymena L-19 IVS RNA can be constructed in which the nucleotide sequence of the active site is complementary to the nucleotide sequence to be cleaved (see Cech et al. U.S. Pat. No. 4,987,071; and Cech et al. U.S. Pat. No. 5,116,742). Alternatively, an mRNA encoding a polypeptide of the invention can be used to select a catalytic RNA having a specific ribonuclease activity from a pool of RNA molecules (see, e.g., Bartel and Szostak, 1993, Science 261:1411-1418).

The invention also encompasses nucleic acid molecules which form triple helical structures. For example, expression of a marker of the invention can be inhibited by targeting nucleotide sequences complementary to the regulatory region of the gene encoding the marker nucleic acid or protein (e.g., the promoter and/or enhancer) to form triple helical structures that prevent transcription of the gene in target cells. See generally Helene (1991) Anticancer Drug Des. 6(6):569-84; Helene (1992) Ann. N.Y. Acad. Sci. 660:27-36; and Maher (1992) Bioassays 14(12):807-15.

In various embodiments, the nucleic acid molecules of the invention can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids (see Hyrup et al., 1996, Bioorganic & Medicinal Chemistry 4(1): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al. (1996), supra; Perry-O'Keefe et al. (1996) Proc. Natl. Acad. Sci. USA 93:14670-675.

PNAs can be used in therapeutic and diagnostic applications. For example, PNAs can be used as antisense or antigene agents for sequence-specific modulation of gene expression by, e.g., inducing transcription or translation arrest or inhibiting replication. PNAs can also be used, e.g., in the analysis of single base pair mutations in a gene by, e.g., PNA directed PCR clamping; as artificial restriction enzymes when used in combination with other enzymes, e.g., S1 nucleases (Hyrup (1996), supra; or as probes or primers for DNA sequence and hybridization (Hyrup, 1996, supra; Perry-O'Keefe et al., 1996, Proc. Natl. Acad. Sci. USA 93:14670-675).

In another embodiment, PNAs can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras can be generated which can combine the advantageous properties of PNA and DNA. Such chimeras allow DNA recognition enzymes, e.g., RNase H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup, 1996, supra). The synthesis of PNA-DNA chimeras can be performed as described in Hyrup (1996), supra, and Finn et al. (1996) Nucleic Acids Res. 24(17):3357-63. For example, a DNA chain can be synthesized on a solid support using standard phosphoramidite coupling chemistry and modified nucleoside analogs. Compounds such as 5′-(4-methoxytrityl)amino-5′-deoxy-thymidine phosphoramidite can be used as a link between the PNA and the 5′ end of DNA (Mag et al., 1989, Nucleic Acids Res. 17:5973-88). PNA monomers are then coupled in a step-wise manner to produce a chimeric molecule with a 5′ PNA segment and a 3′ DNA segment (Finn et al., 1996, Nucleic Acids Res. 24(17):3357-63). Alternatively, chimeric molecules can be synthesized with a 5′ DNA segment and a 3′ PNA segment (Peterser et al., 1975, Bioorganic Med. Chem. Lett. 5:1119-11124).

In other embodiments, the oligonucleotide can include other appended groups such as peptides (e.g., for targeting host cell receptors in vivo), or agents facilitating transport across the cell membrane (see, e.g., Letsinger et al., 1989, Proc. Natl. Acad. Sci. USA 86:6553-6556; Lemaitre et al., 1987, Proc. Natl. Acad. Sci. USA 84:648-652; PCT Publication No. WO 88/09810) or the blood-brain bather (see, e.g., PCT Publication No. WO 89/10134). In addition, oligonucleotides can be modified with hybridization-triggered cleavage agents (see, e.g., Krol et al., 1988, Bio/Techniques 6:958-976) or intercalating agents (see, e.g., Zon, 1988, Pharm. Res. 5:539-549). To this end, the oligonucleotide can be conjugated to another molecule, e.g., a peptide, hybridization triggered cross-linking agent, transport agent, hybridization-triggered cleavage agent, etc.

The invention also includes molecular beacon nucleic acids having at least one region which is complementary to a nucleic acid of the invention, such that the molecular beacon is useful for quantitating the presence of the nucleic acid of the invention in a sample. A “molecular beacon” nucleic acid is a nucleic acid comprising a pair of complementary regions and having a fluorophore and a fluorescent quencher associated therewith. The fluorophore and quencher are associated with different portions of the nucleic acid in such an orientation that when the complementary regions are annealed with one another, fluorescence of the fluorophore is quenched by the quencher. When the complementary regions of the nucleic acid are not annealed with one another, fluorescence of the fluorophore is quenched to a lesser degree. Molecular beacon nucleic acids are described, for example, in U.S. Pat. No. 5,876,930.

### 2. Isolated Proteins and Antibodies

One aspect of the invention pertains to isolated marker proteins and biologically active portions thereof, as well as polypeptide fragments suitable for use as immunogens to raise antibodies directed against a marker protein or a fragment thereof. In one embodiment, the native marker protein can be isolated from cells or tissue sources by an appropriate purification scheme using standard protein purification techniques. In another embodiment, a protein or peptide comprising the whole or a segment of the marker protein is produced by recombinant DNA techniques. Alternative to recombinant expression, such protein or peptide can be synthesized chemically using standard peptide synthesis techniques.

An “isolated” or “purified” protein or biologically active portion thereof is substantially free of cellular material or other contaminating proteins from the cell or tissue source from which the protein is derived, or substantially free of chemical precursors or other chemicals when chemically synthesized. The language “substantially free of cellular material” includes preparations of protein in which the protein is separated from cellular components of the cells from which it is isolated or recombinantly produced. Thus, protein that is substantially free of cellular material includes preparations of protein having less than about 30%, 20%, 10%, or 5% (by dry weight) of heterologous protein (also referred to herein as a “contaminating protein”). When the protein or biologically active portion thereof is recombinantly produced, it is also preferably substantially free of culture medium, i.e., culture medium represents less than about 20%, 10%, or 5% of the volume of the protein preparation. When the protein is produced by chemical synthesis, it is preferably substantially free of chemical precursors or other chemicals, i.e., it is separated from chemical precursors or other chemicals which are involved in the synthesis of the protein. Accordingly such preparations of the protein have less than about 30%, 20%, 10%, 5% (by dry weight) of chemical precursors or compounds other than the polypeptide of interest.

Biologically active portions of a marker protein include polypeptides comprising amino acid sequences sufficiently identical to or derived from the amino acid sequence of the marker protein, which include fewer amino acids than the full length protein, and exhibit at least one activity of the corresponding full-length protein. Typically, biologically active portions comprise a domain or motif with at least one activity of the corresponding full-length protein. A biologically active portion of a marker protein of the invention can be a polypeptide which is, for example, 10, 25, 50, 100 or more amino acids in length. Moreover, other biologically active portions, in which other regions of the marker protein are deleted, can be prepared by recombinant techniques and evaluated for one or more of the functional activities of the native form of the marker protein.

Preferred marker proteins are encoded by nucleotide sequences comprising the sequences encoding any of the genes listed in Tables 2-9. Other useful proteins are substantially identical (e.g., at least about 40%, preferably 50%, 60%, 70%, 80%, 90%, 91%, 92%, 93%, 94%, 95%, 96%, 97%, 98% or 99%) to one of these sequences and retain the functional activity of the corresponding naturally-occurring marker protein yet differ in amino acid sequence due to natural allelic variation or mutagenesis.

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. Preferably, the percent identity between the two sequences is calculated using a global alignment. Alternatively, the percent identity between the two sequences is calculated using a local alignment. The percent identity between the two sequences is a function of the number of identical positions shared by the sequences (i.e., % identity=# of identical positions/total # of positions (e.g., overlapping positions)×100). In one embodiment the two sequences are the same length. In another embodiment, the two sequences are not the same length.

The determination of percent identity between two sequences can be accomplished using a mathematical algorithm. A preferred, non-limiting example of a mathematical algorithm utilized for the comparison of two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the BLASTN and BLASTX programs of Altschul, et al. (1990) J. Mol. Biol. 215:403-410. BLAST nucleotide searches can be performed with the BLASTN program, score=100, wordlength=12 to obtain nucleotide sequences homologous to a nucleic acid molecules of the invention. BLAST protein searches can be performed with the BLASTP program, score=50, wordlength=3 to obtain amino acid sequences homologous to a protein molecules of the invention. To obtain gapped alignments for comparison purposes, a newer version of the BLAST algorithm called Gapped BLAST can be utilized as described in Altschul et al. (1997) Nucleic Acids Res. 25:3389-3402, which is able to perform gapped local alignments for the programs BLASTN, BLASTP and BLASTX. Alternatively, PSI-Blast can be used to perform an iterated search which detects distant relationships between molecules. When utilizing BLAST, Gapped BLAST, and PSI-Blast programs, the default parameters of the respective programs (e.g., BLASTX and BLASTN) can be used. See http://www.ncbi.nlm.nih.gov. Another preferred, non-limiting example of a mathematical algorithm utilized for the comparison of sequences is the algorithm of Myers and Miller, (1988) CABIOS 4:11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Yet another useful algorithm for identifying regions of local sequence similarity and alignment is the FASTA algorithm as described in Pearson and Lipman (1988) Proc. Natl. Acad. Sci. USA 85:2444-2448. When using the FASTA algorithm for comparing nucleotide or amino acid sequences, a PAM120 weight residue table can, for example, be used with a k-tuple value of 2.

The percent identity between two sequences can be determined using techniques similar to those described above, with or without allowing gaps. In calculating percent identity, only exact matches are counted.

The invention also provides chimeric or fusion proteins comprising a marker protein or a segment thereof. As used herein, a “chimeric protein” or “fusion protein” comprises all or part (preferably a biologically active part) of a marker protein operably linked to a heterologous polypeptide (i.e., a polypeptide other than the marker protein). Within the fusion protein, the term “operably linked” is intended to indicate that the marker protein or segment thereof and the heterologous polypeptide are fused in-frame to each other. The heterologous polypeptide can be fused to the amino-terminus or the carboxyl-terminus of the marker protein or segment.

One useful fusion protein is a GST fusion protein in which a marker protein or segment is fused to the carboxyl terminus of GST sequences. Such fusion proteins can facilitate the purification of a recombinant polypeptide of the invention.

In another embodiment, the fusion protein contains a heterologous signal sequence at its amino terminus. For example, the native signal sequence of a marker protein can be removed and replaced with a signal sequence from another protein. For example, the gp67 secretory sequence of the baculovirus envelope protein can be used as a heterologous signal sequence (Ausubel et al., ed., Current Protocols in Molecular Biology, John Wiley & Sons, NY, 1992). Other examples of eukaryotic heterologous signal sequences include the secretory sequences of melittin and human placental alkaline phosphatase (Stratagene; La Jolla, Calif.). In yet another example, useful prokaryotic heterologous signal sequences include the phoA secretory signal (Sambrook et al., supra) and the protein A secretory signal (Pharmacia Biotech; Piscataway, N.J.).

In yet another embodiment, the fusion protein is an immunoglobulin fusion protein in which all or part of a marker protein is fused to sequences derived from a member of the immunoglobulin protein family. The immunoglobulin fusion proteins of the invention can be incorporated into pharmaceutical compositions and administered to a subject to inhibit an interaction between a ligand (soluble or membrane-bound) and a protein on the surface of a cell (receptor), to thereby suppress signal transduction in vivo. The immunoglobulin fusion protein can be used to affect the bioavailability of a cognate ligand of a marker protein Inhibition of ligand/receptor interaction can be useful therapeutically, both for treating proliferative and differentiative disorders and for modulating (e.g. promoting or inhibiting) cell survival. Moreover, the immunoglobulin fusion proteins of the invention can be used as immunogens to produce antibodies directed against a marker protein in a subject, to purify ligands and in screening assays to identify molecules which inhibit the interaction of the marker protein with ligands.

Chimeric and fusion proteins of the invention can be produced by standard recombinant DNA techniques. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and re-amplified to generate a chimeric gene sequence (see, e.g., Ausubel et al., supra). Moreover, many expression vectors are commercially available that already encode a fusion moiety (e.g., a GST polypeptide). A nucleic acid encoding a polypeptide of the invention can be cloned into such an expression vector such that the fusion moiety is linked in-frame to the polypeptide of the invention.

A signal sequence can be used to facilitate secretion and isolation of marker proteins. Signal sequences are typically characterized by a core of hydrophobic amino acids which are generally cleaved from the mature protein during secretion in one or more cleavage events. Such signal peptides contain processing sites that allow cleavage of the signal sequence from the mature proteins as they pass through the secretory pathway. Thus, the invention pertains to marker proteins, fusion proteins or segments thereof having a signal sequence, as well as to such proteins from which the signal sequence has been proteolytically cleaved (i.e., the cleavage products). In one embodiment, a nucleic acid sequence encoding a signal sequence can be operably linked in an expression vector to a protein of interest, such as a marker protein or a segment thereof. The signal sequence directs secretion of the protein, such as from a eukaryotic host into which the expression vector is transformed, and the signal sequence is subsequently or concurrently cleaved. The protein can then be readily purified from the extracellular medium by art recognized methods. Alternatively, the signal sequence can be linked to the protein of interest using a sequence which facilitates purification, such as with a GST domain.

The present invention also pertains to variants of the marker proteins. Such variants have an altered amino acid sequence which can function as either agonists (mimetics) or as antagonists. Variants can be generated by mutagenesis, e.g., discrete point mutation or truncation. An agonist can retain substantially the same, or a subset, of the biological activities of the naturally occurring form of the protein. An antagonist of a protein can inhibit one or more of the activities of the naturally occurring form of the protein by, for example, competitively binding to a downstream or upstream member of a cellular signaling cascade which includes the protein of interest. Thus, specific biological effects can be elicited by treatment with a variant of limited function. Treatment of a subject with a variant having a subset of the biological activities of the naturally occurring form of the protein can have fewer side effects in a subject relative to treatment with the naturally occurring form of the protein.

Variants of a marker protein which function as either agonists (mimetics) or as antagonists can be identified by screening combinatorial libraries of mutants, e.g., truncation mutants, of the protein of the invention for agonist or antagonist activity. In one embodiment, a variegated library of variants is generated by combinatorial mutagenesis at the nucleic acid level and is encoded by a variegated gene library. A variegated library of variants can be produced by, for example, enzymatically ligating a mixture of synthetic oligonucleotides into gene sequences such that a degenerate set of potential protein sequences is expressible as individual polypeptides, or alternatively, as a set of larger fusion proteins (e.g., for phage display). There are a variety of methods which can be used to produce libraries of potential variants of the marker proteins from a degenerate oligonucleotide sequence. Methods for synthesizing degenerate oligonucleotides are known in the art (see, e.g., Narang, 1983, Tetrahedron 39:3; Itakura et al., 1984, Annu. Rev. Biochem. 53:323; Itakura et al., 1984, Science 198:1056; Ike et al., 1983 Nucleic Acid Res. 11:477).

In addition, libraries of segments of a marker protein can be used to generate a variegated population of polypeptides for screening and subsequent selection of variant marker proteins or segments thereof. For example, a library of coding sequence fragments can be generated by treating a double stranded PCR fragment of the coding sequence of interest with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes amino terminal and internal fragments of various sizes of the protein of interest.

Several techniques are known in the art for screening gene products of combinatorial libraries made by point mutations or truncation, and for screening cDNA libraries for gene products having a selected property. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify variants of a protein of the invention (Arkin and Yourvan, 1992, Proc. Natl. Acad. Sci. USA 89:7811-7815; Delgrave et al., 1993, Protein Engineering 6(3):327-331).

Another aspect of the invention pertains to antibodies directed against a protein of the invention. In preferred embodiments, the antibodies specifically bind a marker protein or a fragment thereof. The terms “antibody” and “antibodies” as used interchangeably herein refer to immunoglobulin molecules as well as fragments and derivatives thereof that comprise an immunologically active portion of an immunoglobulin molecule, (i.e., such a portion contains an antigen binding site which specifically binds an antigen, such as a marker protein, e.g., an epitope of a marker protein). An antibody which specifically binds to a protein of the invention is an antibody which binds the protein, but does not substantially bind other molecules in a sample, e.g., a biological sample, which naturally contains the protein. Examples of an immunologically active portion of an immunoglobulin molecule include, but are not limited to, single-chain antibodies (scAb), F(ab) and F(ab′)₂ fragments.

An isolated protein of the invention or a fragment thereof can be used as an immunogen to generate antibodies. The full-length protein can be used or, alternatively, the invention provides antigenic peptide fragments for use as immunogens. The antigenic peptide of a protein of the invention comprises at least 8 (preferably 10, 15, 20, or 30 or more) amino acid residues of the amino acid sequence of one of the proteins of the invention, and encompasses at least one epitope of the protein such that an antibody raised against the peptide forms a specific immune complex with the protein. Preferred epitopes encompassed by the antigenic peptide are regions that are located on the surface of the protein, e.g., hydrophilic regions. Hydrophobicity sequence analysis, hydrophilicity sequence analysis, or similar analyses can be used to identify hydrophilic regions. In preferred embodiments, an isolated marker protein or fragment thereof is used as an immunogen.

An immunogen typically is used to prepare antibodies by immunizing a suitable (i e immunocompetent) subject such as a rabbit, goat, mouse, or other mammal or vertebrate. An appropriate immunogenic preparation can contain, for example, recombinantly-expressed or chemically-synthesized protein or peptide. The preparation can further include an adjuvant, such as Freund's complete or incomplete adjuvant, or a similar immunostimulatory agent. Preferred immunogen compositions are those that contain no other human proteins such as, for example, immunogen compositions made using a non-human host cell for recombinant expression of a protein of the invention. In such a manner, the resulting antibody compositions have reduced or no binding of human proteins other than a protein of the invention.

The invention provides polyclonal and monoclonal antibodies. The term “monoclonal antibody” or “monoclonal antibody composition”, as used herein, refers to a population of antibody molecules that contain only one species of an antigen binding site capable of immunoreacting with a particular epitope. Preferred polyclonal and monoclonal antibody compositions are ones that have been selected for antibodies directed against a protein of the invention. Particularly preferred polyclonal and monoclonal antibody preparations are ones that contain only antibodies directed against a marker protein or fragment thereof.

Polyclonal antibodies can be prepared by immunizing a suitable subject with a protein of the invention as an immunogen The antibody titer in the immunized subject can be monitored over time by standard techniques, such as with an enzyme linked immunosorbent assay (ELISA) using immobilized polypeptide. At an appropriate time after immunization, e.g., when the specific antibody titers are highest, antibody-producing cells can be obtained from the subject and used to prepare monoclonal antibodies (mAb) by standard techniques, such as the hybridoma technique originally described by Kohler and Milstein (1975) Nature 256:495-497, the human B cell hybridoma technique (see Kozbor et al., 1983, Immunol. Today 4:72), the EBV-hybridoma technique (see Cole et al., pp. 77-96 In Monoclonal Antibodies and Cancer Therapy, Alan R. Liss, Inc., 1985) or trioma techniques. The technology for producing hybridomas is well known (see generally Current Protocols in Immunology, Coligan et al. ed., John Wiley & Sons, New York, 1994). Hybridoma cells producing a monoclonal antibody of the invention are detected by screening the hybridoma culture supernatants for antibodies that bind the polypeptide of interest, e.g., using a standard ELISA assay.

Alternative to preparing monoclonal antibody-secreting hybridomas, a monoclonal antibody directed against a protein of the invention can be identified and isolated by screening a recombinant combinatorial immunoglobulin library (e.g., an antibody phage display library) with the polypeptide of interest. Kits for generating and screening phage display libraries are commercially available (e.g., the Pharmacia Recombinant Phage Antibody System, Catalog No. 27-9400-01; and the Stratagene SurfZAP Phage Display Kit, Catalog No. 240612). Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J. 12:725-734.

The invention also provides recombinant antibodies that specifically bind a protein of the invention. In preferred embodiments, the recombinant antibodies specifically binds a marker protein or fragment thereof. Recombinant antibodies include, but are not limited to, chimeric and humanized monoclonal antibodies, comprising both human and non-human portions, single-chain antibodies and multi-specific antibodies. A chimeric antibody is a molecule in which different portions are derived from different animal species, such as those having a variable region derived from a murine mAb and a human immunoglobulin constant region. (See, e.g., Cabilly et al., U.S. Pat. No. 4,816,567; and Boss et al., U.S. Pat. No. 4,816,397, which are incorporated herein by reference in their entirety.) Single-chain antibodies have an antigen binding site and consist of a single polypeptide. They can be produced by techniques known in the art, for example using methods described in Ladner et. al. U.S. Pat. No. 4,946,778 (which is incorporated herein by reference in its entirety); Bird et al., (1988) Science 242:423-426; Whitlow et al., (1991) Methods in Enzymology 2:1-9; Whitlow et al., (1991) Methods in Enzymology 2:97-105; and Huston et al., (1991) Methods in Enzymology Molecular Design and Modeling: Concepts and Applications 203:46-88. Multi-specific antibodies are antibody molecules having at least two antigen-binding sites that specifically bind different antigens. Such molecules can be produced by techniques known in the art, for example using methods described in Segal, U.S. Pat. No. 4,676,980 (the disclosure of which is incorporated herein by reference in its entirety); Holliger et al., (1993) Proc. Natl. Acad. Sci. USA 90:6444-6448; Whitlow et al., (1994) Protein Eng. 7:1017-1026 and U.S. Pat. No. 6,121,424.

Humanized antibodies are antibody molecules from non-human species having one or more complementarity determining regions (CDRs) from the non-human species and a framework region from a human immunoglobulin molecule. (See, e.g., Queen, U.S. Pat. No. 5,585,089, which is incorporated herein by reference in its entirety.) Humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. No. 4,816,567; European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Cancer Res. 47:999-1005; Wood et al. (1985) Nature 314:446-449; and Shaw et al. (1988) J. Natl. Cancer Inst. 80:1553-1559); Morrison (1985) Science 229:1202-1207; Oi et al. (1986) Bio/Techniques 4:214; U.S. Pat. No. 5,225,539; Jones et al. (1986) Nature 321:552-525; Verhoeyan et al. (1988) Science 239:1534; and Beidler et al. (1988) J. Immunol. 141:4053-4060.

More particularly, humanized antibodies can be produced, for example, using transgenic mice which are incapable of expressing endogenous immunoglobulin heavy and light chains genes, but which can express human heavy and light chain genes. The transgenic mice are immunized in the normal fashion with a selected antigen, e.g., all or a portion of a polypeptide corresponding to a marker of the invention. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA and IgE antibodies. For an overview of this technology for producing human antibodies, see Lonberg and Huszar (1995) Int. Rev. Immunol. 13:65-93). For a detailed discussion of this technology for producing human antibodies and human monoclonal antibodies and protocols for producing such antibodies, see, e.g., U.S. Pat. No. 5,625,126; U.S. Pat. No. 5,633,425; U.S. Pat. No. 5,569,825; U.S. Pat. No. 5,661,016; and U.S. Pat. No. 5,545,806. In addition, companies such as Abgenix, Inc. (Freemont, Calif.), can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a murine antibody, is used to guide the selection of a completely human antibody recognizing the same epitope (Jespers et al., 1994, Bio/technology 12:899-903).

The antibodies of the invention can be isolated after production (e.g., from the blood or serum of the subject) or synthesis and further purified by well-known techniques. For example, IgG antibodies can be purified using protein A chromatography. Antibodies specific for a protein of the invention can be selected or (e.g., partially purified) or purified by, e.g., affinity chromatography. For example, a recombinantly expressed and purified (or partially purified) protein of the invention is produced as described herein, and covalently or non-covalently coupled to a solid support such as, for example, a chromatography column. The column can then be used to affinity purify antibodies specific for the proteins of the invention from a sample containing antibodies directed against a large number of different epitopes, thereby generating a substantially purified antibody composition, i.e., one that is substantially free of contaminating antibodies. By a substantially purified antibody composition is meant, in this context, that the antibody sample contains at most only 30% (by dry weight) of contaminating antibodies directed against epitopes other than those of the desired protein of the invention, and preferably at most 20%, yet more preferably at most 10%, and most preferably at most 5% (by dry weight) of the sample is contaminating antibodies. A purified antibody composition means that at least 99% of the antibodies in the composition are directed against the desired protein of the invention.

In a preferred embodiment, the substantially purified antibodies of the invention may specifically bind to a signal peptide, a secreted sequence, an extracellular domain, a transmembrane or a cytoplasmic domain or cytoplasmic membrane of a protein of the invention. In a particularly preferred embodiment, the substantially purified antibodies of the invention specifically bind to a secreted sequence or an extracellular domain of the amino acid sequences of a protein of the invention. In a more preferred embodiment, the substantially purified antibodies of the invention specifically bind to a secreted sequence or an extracellular domain of the amino acid sequences of a marker protein.

An antibody directed against a protein of the invention can be used to isolate the protein by standard techniques, such as affinity chromatography or immunoprecipitation. Moreover, such an antibody can be used to detect the marker protein or fragment thereof (e.g., in a cellular lysate or cell supernatant) in order to evaluate the level and pattern of expression of the marker. The antibodies can also be used diagnostically to monitor protein levels in tissues or body fluids (e.g. in sarcoma-associated body fluid) as part of a clinical testing procedure, e.g., to, for example, determine the efficacy of a given treatment regimen. Detection can be facilitated by the use of an antibody derivative, which comprises an antibody of the invention coupled to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Antibodies of the invention may also be used as therapeutic agents in treating cancers. In a preferred embodiment, completely human antibodies of the invention are used for therapeutic treatment of human cancer patients, particularly those having a cancer. In another preferred embodiment, antibodies that bind specifically to a marker protein or fragment thereof are used for therapeutic treatment. Further, such therapeutic antibody may be an antibody derivative or immunotoxin comprising an antibody conjugated to a therapeutic moiety such as a cytotoxin, a therapeutic agent or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

The conjugated antibodies of the invention can be used for modifying a given biological response, for the drug moiety is not to be construed as limited to classical chemical therapeutic agents. For example, the drug moiety may be a protein or polypeptide possessing a desired biological activity. Such proteins may include, for example, a toxin such as ribosome-inhibiting protein (see Better et al., U.S. Pat. No. 6,146,631, the disclosure of which is incorporated herein in its entirety), abrin, ricin A, pseudomonas exotoxin, or diphtheria toxin; a protein such as tumor necrosis factor, .alpha.-interferon, β-interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophase colony stimulating factor (“GM-CSF”), granulocyte colony stimulating factor (“G-CSF”), or other growth factors.

Techniques for conjugating such therapeutic moiety to antibodies are well known, see, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982).

Accordingly, in one aspect, the invention provides substantially purified antibodies, antibody fragments and derivatives, all of which specifically bind to a protein of the invention and preferably, a marker protein. In various embodiments, the substantially purified antibodies of the invention, or fragments or derivatives thereof, can be human, non-human, chimeric and/or humanized antibodies. In another aspect, the invention provides non-human antibodies, antibody fragments and derivatives, all of which specifically bind to a protein of the invention and preferably, a marker protein. Such non-human antibodies can be goat, mouse, sheep, horse, chicken, rabbit, or rat antibodies. Alternatively, the non-human antibodies of the invention can be chimeric and/or humanized antibodies. In addition, the non-human antibodies of the invention can be polyclonal antibodies or monoclonal antibodies. In still a further aspect, the invention provides monoclonal antibodies, antibody fragments and derivatives, all of which specifically bind to a protein of the invention and preferably, a marker protein. The monoclonal antibodies can be human, humanized, chimeric and/or non-human antibodies.

The invention also provides a kit containing an antibody of the invention conjugated to a detectable substance, and instructions for use. Still another aspect of the invention is a pharmaceutical composition comprising an antibody of the invention. In one embodiment, the pharmaceutical composition comprises an antibody of the invention and a pharmaceutically acceptable carrier.

### 3. Predictive Medicine

The present invention pertains to the field of predictive medicine in which diagnostic assays, prognostic assays, pharmacogenomics, and monitoring clinical trails are used for prognostic (predictive) purposes to thereby treat an individual prophylactically. Accordingly, one aspect of the present invention relates to diagnostic assays for determining the level of expression of one or more marker proteins or nucleic acids, in order to determine whether an individual is at risk of developing a sarcoma. Such assays can be used for prognostic or predictive purposes to thereby prophylactically treat an individual prior to the onset of the disorder.

Yet another aspect of the invention pertains to monitoring the influence of agents (e.g., drugs or other compounds administered either to inhibit a sarcoma or to treat or prevent any other disorder {i.e. in order to understand any carcinogenic effects that such treatment may have}) on the expression or activity of a marker of the invention in clinical trials. These and other agents are described in further detail in the following sections.

A. Diagnostic Assays

An exemplary method for detecting the presence or absence of a marker protein or nucleic acid in a biological sample involves obtaining a biological sample (e.g. sarcoma-associated body fluid or tissue sample) from a test subject and contacting the biological sample with a compound or an agent capable of detecting the polypeptide or nucleic acid (e.g., mRNA, genomic DNA, or cDNA). The detection methods of the invention can thus be used to detect mRNA, protein, cDNA, or genomic DNA, for example, in a biological sample in vitro as well as in vivo. For example, in vitro techniques for detection of mRNA include Northern hybridizations and in situ hybridizations. In vitro techniques for detection of a marker protein include enzyme linked immunosorbent assays (ELISAs), Western blots, immunoprecipitations and immunofluorescence. In vitro techniques for detection of genomic DNA include Southern hybridizations. In vivo techniques for detection of mRNA include polymerase chain reaction (PCR), Northern hybridizations and in situ hybridizations. Furthermore, in vivo techniques for detection of a marker protein include introducing into a subject a labeled antibody directed against the protein or fragment thereof. For example, the antibody can be labeled with a radioactive marker whose presence and location in a subject can be detected by standard imaging techniques.

A general principle of such diagnostic and prognostic assays involves preparing a sample or reaction mixture that may contain a marker, and a probe, under appropriate conditions and for a time sufficient to allow the marker and probe to interact and bind, thus forming a complex that can be removed and/or detected in the reaction mixture. These assays can be conducted in a variety of ways.

For example, one method to conduct such an assay would involve anchoring the marker or probe onto a solid phase support, also referred to as a substrate, and detecting target marker/probe complexes anchored on the solid phase at the end of the reaction. In one embodiment of such a method, a sample from a subject, which is to be assayed for presence and/or concentration of marker, can be anchored onto a carrier or solid phase support. In another embodiment, the reverse situation is possible, in which the probe can be anchored to a solid phase and a sample from a subject can be allowed to react as an unanchored component of the assay.

There are many established methods for anchoring assay components to a solid phase. These include, without limitation, marker or probe molecules which are immobilized through conjugation of biotin and streptavidin. Such biotinylated assay components can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical). In certain embodiments, the surfaces with immobilized assay components can be prepared in advance and stored.

Other suitable carriers or solid phase supports for such assays include any material capable of binding the class of molecule to which the marker or probe belongs. Well-known supports or carriers include, but are not limited to, glass, polystyrene, nylon, polypropylene, nylon, polyethylene, dextran, amylases, natural and modified celluloses, polyacrylamides, gabbros, and magnetite.

In order to conduct assays with the above mentioned approaches, the non-immobilized component is added to the solid phase upon which the second component is anchored. After the reaction is complete, uncomplexed components may be removed (e.g., by washing) under conditions such that any complexes formed will remain immobilized upon the solid phase. The detection of marker/probe complexes anchored to the solid phase can be accomplished in a number of methods outlined herein.

In a preferred embodiment, the probe, when it is the unanchored assay component, can be labeled for the purpose of detection and readout of the assay, either directly or indirectly, with detectable labels discussed herein and which are well-known to one skilled in the art.

It is also possible to directly detect marker/probe complex formation without further manipulation or labeling of either component (marker or probe), for example by utilizing the technique of fluorescence energy transfer (see, for example, Lakowicz et al., U.S. Pat. No. 5,631,169; Stavrianopoulos, et al., U.S. Pat. No. 4,868,103). A fluorophore label on the first, ‘donor’ molecule is selected such that, upon excitation with incident light of appropriate wavelength, its emitted fluorescent energy will be absorbed by a fluorescent label on a second ‘acceptor’ molecule, which in turn is able to fluoresce due to the absorbed energy. Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, spatial relationships between the molecules can be assessed. In a situation in which binding occurs between the molecules, the fluorescent emission of the ‘acceptor’ molecule label in the assay should be maximal. An FET binding event can be conveniently measured through standard fluorometric detection means well known in the art (e.g., using a fluorimeter).

In another embodiment, determination of the ability of a probe to recognize a marker can be accomplished without labeling either assay component (probe or marker) by utilizing a technology such as real-time Biomolecular Interaction Analysis (BIA) (see, e.g., Sjolander, S, and Urbaniczky, C., 1991, Anal. Chem. 63:2338-2345 and Szabo et al., 1995, Curr. Opin. Struct. Biol. 5:699-705). As used herein, “BIA” or “surface plasmon resonance” is a technology for studying biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules.

Alternatively, in another embodiment, analogous diagnostic and prognostic assays can be conducted with marker and probe as solutes in a liquid phase. In such an assay, the complexed marker and probe are separated from uncomplexed components by any of a number of standard techniques, including but not limited to: differential centrifugation, chromatography, electrophoresis and immunoprecipitation. In differential centrifugation, marker/probe complexes may be separated from uncomplexed assay components through a series of centrifugal steps, due to the different sedimentation equilibria of complexes based on their different sizes and densities (see, for example, Rivas, G., and Minton, A. P., 1993, Trends Biochem Sci. 18(8):284-7). Standard chromatographic techniques may also be utilized to separate complexed molecules from uncomplexed ones. For example, gel filtration chromatography separates molecules based on size, and through the utilization of an appropriate gel filtration resin in a column format, for example, the relatively larger complex may be separated from the relatively smaller uncomplexed components. Similarly, the relatively different charge properties of the marker/probe complex as compared to the uncomplexed components may be exploited to differentiate the complex from uncomplexed components, for example through the utilization of ion-exchange chromatography resins. Such resins and chromatographic techniques are well known to one skilled in the art (see, e.g., Heegaard, N. H., 1998, J. Mol. Recognit. Winter 11(1-6):141-8; Hage, D. S., and Tweed, S. A. J Chromatogr B Biomed Sci Appl 1997 Oct. 10; 699(1-2):499-525). Gel electrophoresis may also be employed to separate complexed assay components from unbound components (see, e.g., Ausubel et al., ed., Current Protocols in Molecular Biology, John Wiley & Sons, New York, 1987-1999). In this technique, protein or nucleic acid complexes are separated based on size or charge, for example. In order to maintain the binding interaction during the electrophoretic process, non-denaturing gel matrix materials and conditions in the absence of reducing agent are typically preferred. Appropriate conditions to the particular assay and components thereof will be well known to one skilled in the art.

In a particular embodiment, the level of marker mRNA can be determined both by in situ and by in vitro formats in a biological sample using methods known in the art. The term “biological sample” is intended to include tissues, cells, biological fluids and isolates thereof, isolated from a subject, as well as tissues, cells and fluids present within a subject. Many expression detection methods use isolated RNA. For in vitro methods, any RNA isolation technique that does not select against the isolation of mRNA can be utilized for the purification of RNA from cells (see, e.g., Ausubel et al., ed., Current Protocols in Molecular Biology, John Wiley & Sons, New York 1987-1999). Additionally, large numbers of tissue samples can readily be processed using techniques well known to those of skill in the art, such as, for example, the single-step RNA isolation process of Chomczynski (1989, U.S. Pat. No. 4,843,155).

The isolated mRNA can be used in hybridization or amplification assays that include, but are not limited to, Southern or Northern analyses, polymerase chain reaction analyses and probe arrays. One preferred diagnostic method for the detection of mRNA levels involves contacting the isolated mRNA with a nucleic acid molecule (probe) that can hybridize to the mRNA encoded by the gene being detected. The nucleic acid probe can be, for example, a full-length cDNA, or a portion thereof, such as an oligonucleotide of at least 7, 15, 30, 50, 100, 250 or 500 nucleotides in length and sufficient to specifically hybridize under stringent conditions to a mRNA or genomic DNA encoding a marker of the present invention. Other suitable probes for use in the diagnostic assays of the invention are described herein. Hybridization of an mRNA with the probe indicates that the marker in question is being expressed.

In one format, the mRNA is immobilized on a solid surface and contacted with a probe, for example by running the isolated mRNA on an agarose gel and transferring the mRNA from the gel to a membrane, such as nitrocellulose. In an alternative format, the probe(s) are immobilized on a solid surface and the mRNA is contacted with the probe(s), for example, in an Affymetrix gene chip array. A skilled artisan can readily adapt known mRNA detection methods for use in detecting the level of mRNA encoded by the markers of the present invention.

An alternative method for determining the level of mRNA marker in a sample involves the process of nucleic acid amplification, e.g., by RT-PCR (the experimental embodiment set forth in Mullis, 1987, U.S. Pat. No. 4,683,202), ligase chain reaction (Barany, 1991, Proc. Natl. Acad. Sci. USA, 88:189-193), self sustained sequence replication (Guatelli et al., 1990, Proc. Natl. Acad. Sci. USA 87:1874-1878), transcriptional amplification system (Kwoh et al., 1989, Proc. Natl. Acad. Sci. USA 86:1173-1177), Q-Beta Replicase (Lizardi et al., 1988, Bio/Technology 6:1197), rolling circle replication (Lizardi et al., U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

For in situ methods, mRNA does not need to be isolated from the prior to detection. In such methods, a cell or tissue sample is prepared/processed using known histological methods. The sample is then immobilized on a support, typically a glass slide, and then contacted with a probe that can hybridize to mRNA that encodes the marker.

As an alternative to making determinations based on the absolute expression level of the marker, determinations may be based on the normalized expression level of the marker. Expression levels are normalized by correcting the absolute expression level of a marker by comparing its expression to the expression of a gene that is not a marker, e.g., a housekeeping gene that is constitutively expressed. Suitable genes for normalization include housekeeping genes such as the actin gene, or epithelial cell-specific genes. This normalization allows the comparison of the expression level in one sample, e.g., a patient sample, to another sample, e.g., a non-cancer sample, or between samples from different sources.

Alternatively, the expression level can be provided as a relative expression level. To determine a relative expression level of a marker, the level of expression of the marker is determined for 10 or more samples of normal versus cancer cell isolates, preferably 50 or more samples, prior to the determination of the expression level for the sample in question. The mean expression level of each of the genes assayed in the larger number of samples is determined and this is used as a baseline expression level for the marker. The expression level of the marker determined for the test sample (absolute level of expression) is then divided by the mean expression value obtained for that marker. This provides a relative expression level.

Preferably, the samples used in the baseline determination will be from non-cancer cells. The choice of the cell source is dependent on the use of the relative expression level. Using expression found in normal tissues as a mean expression score aids in validating whether the marker assayed is cancer specific (versus normal cells). In addition, as more data is accumulated, the mean expression value can be revised, providing improved relative expression values based on accumulated data. Expression data from cancer cells provides a means for grading the severity of the cancer state.

In another embodiment of the present invention, a marker protein is detected. A preferred agent for detecting marker protein of the invention is an antibody capable of binding to such a protein or a fragment thereof, preferably an antibody with a detectable label. Antibodies can be polyclonal, or more preferably, monoclonal. An intact antibody, or a fragment or derivative thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

Proteins from cells can be isolated using techniques that are well known to those of skill in the art. The protein isolation methods employed can, for example, be such as those described in Harlow and Lane (Harlow and Lane, 1988, Antibodies: A Laboratory Manual, Cold Spring Harbor Laboratory Press, Cold Spring Harbor, N.Y.).

A variety of formats can be employed to determine whether a sample contains a protein that binds to a given antibody. Examples of such formats include, but are not limited to, enzyme immunoassay (EIA), radioimmunoassay (RIA), Western blot analysis and enzyme linked immunoabsorbant assay (ELISA). A skilled artisan can readily adapt known protein/antibody detection methods for use in determining whether cells express a marker of the present invention.

In one format, antibodies, or antibody fragments or derivatives, can be used in methods such as Western blots or immunofluorescence techniques to detect the expressed proteins. In such uses, it is generally preferable to immobilize either the antibody or proteins on a solid support. Suitable solid phase supports or carriers include any support capable of binding an antigen or an antibody. Well-known supports or carriers include glass, polystyrene, polypropylene, polyethylene, dextran, nylon, amylases, natural and modified celluloses, polyacrylamides, gabbros, and magnetite.

One skilled in the art will know many other suitable carriers for binding antibody or antigen, and will be able to adapt such support for use with the present invention. For example, protein isolated from cancer cells can be run on a polyacrylamide gel electrophoresis and immobilized onto a solid phase support such as nitrocellulose. The support can then be washed with suitable buffers followed by treatment with the detectably labeled antibody. The solid phase support can then be washed with the buffer a second time to remove unbound antibody. The amount of bound label on the solid support can then be detected by conventional means.

The invention also encompasses kits for detecting the presence of a marker protein or nucleic acid in a biological sample. Such kits can be used to determine if a subject is suffering from or is at increased risk of developing sarcoma. For example, the kit can comprise a labeled compound or agent capable of detecting a marker protein or nucleic acid in a biological sample and means for determining the amount of the protein or mRNA in the sample (e.g., an antibody which binds the protein or a fragment thereof, or an oligonucleotide probe which binds to DNA or mRNA encoding the protein). Kits can also include instructions for interpreting the results obtained using the kit.

For antibody-based kits, the kit can comprise, for example: (1) a first antibody (e.g., attached to a solid support) which binds to a marker protein; and, optionally, (2) a second, different antibody which binds to either the protein or the first antibody and is conjugated to a detectable label.

For oligonucleotide-based kits, the kit can comprise, for example: (1) an oligonucleotide, e.g., a detectably labeled oligonucleotide, which hybridizes to a nucleic acid sequence encoding a marker protein or (2) a pair of primers useful for amplifying a marker nucleic acid molecule. The kit can also comprise, e.g., a buffering agent, a preservative, or a protein stabilizing agent. The kit can further comprise components necessary for detecting the detectable label (e.g., an enzyme or a substrate). The kit can also contain a control sample or a series of control samples which can be assayed and compared to the test sample. Each component of the kit can be enclosed within an individual container and all of the various containers can be within a single package, along with instructions for interpreting the results of the assays performed using the kit.

B. Pharmacogenomics

The markers of the invention are also useful as pharmacogenomic markers. As used herein, a “pharmacogenomic marker” is an objective biochemical marker whose expression level correlates with a specific clinical drug response or susceptibility in a patient (see, e.g., McLeod et al. (1999) Eur. J. Cancer 35(12): 1650-1652). The presence or quantity of the pharmacogenomic marker expression is related to the predicted response of the patient and more particularly the patient's a sarcoma to therapy with a specific drug or class of drugs. By assessing the presence or quantity of the expression of one or more pharmacogenomic markers in a patient, a drug therapy which is most appropriate for the patient, or which is predicted to have a greater degree of success, may be selected. For example, based on the presence or quantity of RNA or protein encoded by specific tumor markers in a patient, a drug or course of treatment may be selected that is optimized for the treatment of the specific tumor likely to be present in the patient. The use of pharmacogenomic markers therefore permits selecting or designing the most appropriate treatment for each cancer patient without trying different drugs or regimes.

Another aspect of pharmacogenomics deals with genetic conditions that alters the way the body acts on drugs. These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, a PM will show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Thus, the level of expression of a marker of the invention in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with a modulator of expression of a marker of the invention.

C. Monitoring Clinical Trials

Monitoring the influence of agents (e.g., drug compounds) on the level of expression of a marker of the invention can be applied not only in basic drug screening, but also in clinical trials. For example, the effectiveness of an agent to affect marker expression can be monitored in clinical trials of subjects receiving treatment for a sarcoma. In a preferred embodiment, the present invention provides a method for monitoring the effectiveness of treatment of a subject with an agent (e.g., an agonist, antagonist, peptidomimetic, protein, peptide, nucleic acid, small molecule, or other drug candidate) comprising the steps of (i) obtaining a pre-administration sample from a subject prior to administration of the agent; (ii) detecting the level of expression of one or more selected markers of the invention in the pre-administration sample; (iii) obtaining one or more post-administration samples from the subject; (iv) detecting the level of expression of the marker(s) in the post-administration samples; (v) comparing the level of expression of the marker(s) in the pre-administration sample with the level of expression of the marker(s) in the post-administration sample or samples; and (vi) altering the administration of the agent to the subject accordingly. For example, increased expression of the marker gene(s) during the course of treatment may indicate ineffective dosage and the desirability of increasing the dosage. Conversely, decreased expression of the marker gene(s) may indicate efficacious treatment and no need to change dosage.

D. Arrays

The invention also includes an array comprising a marker of the present invention. The array can be used to assay expression of one or more genes in the array. In one embodiment, the array can be used to assay gene expression in a tissue to ascertain tissue specificity of genes in the array. In this manner, up to about 7600 genes can be simultaneously assayed for expression. This allows a profile to be developed showing a battery of genes specifically expressed in one or more tissues.

In addition to such qualitative determination, the invention allows the quantitation of gene expression. Thus, not only tissue specificity, but also the level of expression of a battery of genes in the tissue is ascertainable. Thus, genes can be grouped on the basis of their tissue expression per se and level of expression in that tissue. This is useful, for example, in ascertaining the relationship of gene expression between or among tissues. Thus, one tissue can be perturbed and the effect on gene expression in a second tissue can be determined. In this context, the effect of one cell type on another cell type in response to a biological stimulus can be determined. Such a determination is useful, for example, to know the effect of cell-cell interaction at the level of gene expression. If an agent is administered therapeutically to treat one cell type but has an undesirable effect on another cell type, the invention provides an assay to determine the molecular basis of the undesirable effect and thus provides the opportunity to co-administer a counteracting agent or otherwise treat the undesired effect. Similarly, even within a single cell type, undesirable biological effects can be determined at the molecular level. Thus, the effects of an agent on expression of other than the target gene can be ascertained and counteracted.

In another embodiment, the array can be used to monitor the time course of expression of one or more genes in the array. This can occur in various biological contexts, as disclosed herein, for example development of sarcoma, progression of sarcoma, and processes, such a cellular transformation associated with sarcoma.

The array is also useful for ascertaining the effect of the expression of a gene on the expression of other genes in the same cell or in different cells. This provides, for example, for a selection of alternate molecular targets for therapeutic intervention if the ultimate or downstream target cannot be regulated.

The array is also useful for ascertaining differential expression patterns of one or more genes in normal and abnormal cells. This provides a battery of genes that could serve as a molecular target for diagnosis or therapeutic intervention.

### VII. Methods for Obtaining Samples

Samples useful in the methods of the invention include any tissue, cell, biopsy, or bodily fluid sample that expresses a marker of the invention. In one embodiment, a sample may be a tissue, a cell, whole blood, serum, plasma, buccal scrape, saliva, cerebrospinal fluid, urine, stool, or bronchoalveolar lavage. In preferred embodiments, the tissue sample is a sarcoma sample.

Body samples may be obtained from a subject by a variety of techniques known in the art including, for example, by the use of a biopsy or by scraping or swabbing an area or by using a needle to aspirate bodily fluids. Methods for collecting various body samples are well known in the art.

Tissue samples suitable for detecting and quantitating a marker of the invention may be fresh, frozen, or fixed according to methods known to one of skill in the art. Suitable tissue samples are preferably sectioned and placed on a microscope slide for further analyses. Alternatively, solid samples, i.e., tissue samples, may be solubilized and/or homogenized and subsequently analyzed as soluble extracts.

In one embodiment, a freshly obtained biopsy sample is frozen using, for example, liquid nitrogen or difluorodichloromethane. The frozen sample is mounted for sectioning using, for example, OCT, and serially sectioned in a cryostat. The serial sections are collected on a glass microscope slide. For immunohistochemical staining the slides may be coated with, for example, chrome-alum, gelatine or poly-L-lysine to ensure that the sections stick to the slides. In another embodiment, samples are fixed and embedded prior to sectioning. For example, a tissue sample may be fixed in, for example, formalin, serially dehydrated and embedded in, for example, paraffin.

Once the sample is obtained any method known in the art to be suitable for detecting and quantitating a marker of the invention may be used (either at the nucleic acid or at the protein level). Such methods are well known in the art and include but are not limited to western blots, northern blots, southern blots, immunohistochemistry, ELISA, e.g., amplified ELISA, immunoprecipitation, immunofluorescence, flow cytometry, immunocytochemistry, mass spectrometrometric analyses, e.g., MALDI-TOF and SELDI-TOF, nucleic acid hybridization techniques, nucleic acid reverse transcription methods, and nucleic acid amplification methods. In particular embodiments, the expression of a marker of the invention is detected on a protein level using, for example, antibodies that specifically bind these proteins.

Samples may need to be modified in order to make a marker of the invention accessible to antibody binding. In a particular aspect of the immunocytochemistry or immunohistochemistry methods, slides may be transferred to a pretreatment buffer and optionally heated to increase antigen accessibility. Heating of the sample in the pretreatment buffer rapidly disrupts the lipid bi-layer of the cells and makes the antigens (may be the case in fresh specimens, but not typically what occurs in fixed specimens) more accessible for antibody binding. The terms “pretreatment buffer” and “preparation buffer” are used interchangeably herein to refer to a buffer that is used to prepare cytology or histology samples for immunostaining, particularly by increasing the accessibility of a marker of the invention for antibody binding. The pretreatment buffer may comprise a pH-specific salt solution, a polymer, a detergent, or a nonionic or anionic surfactant such as, for example, an ethyloxylated anionic or nonionic surfactant, an alkanoate or an alkoxylate or even blends of these surfactants or even the use of a bile salt. The pretreatment buffer may, for example, be a solution of 0.1% to 1% of deoxycholic acid, sodium salt, or a solution of sodium laureth-13-carboxylate (e.g., Sandopan LS) or and ethoxylated anionic complex. In some embodiments, the pretreatment buffer may also be used as a slide storage buffer.

Any method for making marker proteins of the invention more accessible for antibody binding may be used in the practice of the invention, including the antigen retrieval methods known in the art. See, for example, Bibbo, et al. (2002) Acta. Cytol. 46:25-29; Saqi, et al. (2003) Diagn. Cytopathol. 27:365-370; Bibbo, et al. (2003) Anal. Quant. Cytol. Histol. 25:8-11, the entire contents of each of which are incorporated herein by reference.

Following pretreatment to increase marker protein accessibility, samples may be blocked using an appropriate blocking agent, e.g., a peroxidase blocking reagent such as hydrogen peroxide. In some embodiments, the samples may be blocked using a protein blocking reagent to prevent non-specific binding of the antibody. The protein blocking reagent may comprise, for example, purified casein. An antibody, particularly a monoclonal or polyclonal antibody that specifically binds to a marker of the invention is then incubated with the sample. One of skill in the art will appreciate that a more accurate prognosis or diagnosis may be obtained in some cases by detecting multiple epitopes on a marker protein of the invention in a patient sample. Therefore, in particular embodiments, at least two antibodies directed to different epitopes of a marker of the invention are used. Where more than one antibody is used, these antibodies may be added to a single sample sequentially as individual antibody reagents or simultaneously as an antibody cocktail. Alternatively, each individual antibody may be added to a separate sample from the same patient, and the resulting data pooled.

Techniques for detecting antibody binding are well known in the art. Antibody binding to a marker of the invention may be detected through the use of chemical reagents that generate a detectable signal that corresponds to the level of antibody binding and, accordingly, to the level of marker protein expression. In one of the immunohistochemistry or immunocytochemistry methods of the invention, antibody binding is detected through the use of a secondary antibody that is conjugated to a labeled polymer. Examples of labeled polymers include but are not limited to polymer-enzyme conjugates. The enzymes in these complexes are typically used to catalyze the deposition of a chromogen at the antigen-antibody binding site, thereby resulting in cell staining that corresponds to expression level of the biomarker of interest. Enzymes of particular interest include, but are not limited to, horseradish peroxidase (HRP) and alkaline phosphatase (AP).

In one particular immunohistochemistry or immunocytochemistry method of the invention, antibody binding to a marker of the invention is detected through the use of an HRP-labeled polymer that is conjugated to a secondary antibody. Antibody binding can also be detected through the use of a species-specific probe reagent, which binds to monoclonal or polyclonal antibodies, and a polymer conjugated to HRP, which binds to the species specific probe reagent. Slides are stained for antibody binding using any chromagen, e.g., the chromagen 3,3-diaminobenzidine (DAB), and then counterstained with hematoxylin and, optionally, a bluing agent such as ammonium hydroxide or TBS/Tween-20. Other suitable chromagens include, for example, 3-amino-9-ethylcarbazole (AEC). In some aspects of the invention, slides are reviewed microscopically by a cytotechnologist and/or a pathologist to assess cell staining, e.g., fluorescent staining (i.e., marker expression). Alternatively, samples may be reviewed via automated microscopy or by personnel with the assistance of computer software that facilitates the identification of positive staining cells.

Detection of antibody binding can be facilitated by coupling the anti-marker antibodies to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include ¹²⁵I, ¹³¹I, ³⁵S, ¹⁴C, or ³H.

In one embodiment of the invention frozen samples are prepared as described above and subsequently stained with antibodies against a marker of the invention diluted to an appropriate concentration using, for example, Tris-buffered saline (TBS). Primary antibodies can be detected by incubating the slides in biotinylated anti-immunoglobulin. This signal can optionally be amplified and visualized using diaminobenzidine precipitation of the antigen. Furthermore, slides can be optionally counterstained with, for example, hematoxylin, to visualize the cells.

In another embodiment, fixed and embedded samples are stained with antibodies against a marker of the invention and counterstained as described above for frozen sections. In addition, samples may be optionally treated with agents to amplify the signal in order to visualize antibody staining. For example, a peroxidase-catalyzed deposition of biotinyl-tyramide, which in turn is reacted with peroxidase-conjugated streptavidin (Catalyzed Signal Amplification (CSA) System, DAKO, Carpinteria, Calif.) may be used.

Tissue-based assays (i.e., immunohistochemistry) are the preferred methods of detecting and quantitating a marker of the invention. In one embodiment, the presence or absence of a marker of the invention may be determined by immunohistochemistry. In one embodiment, the immunohistochemical analysis uses low concentrations of an anti-marker antibody such that cells lacking the marker do not stain. In another embodiment, the presence or absence of a marker of the invention is determined using an immunohistochemical method that uses high concentrations of an anti-marker antibody such that cells lacking the marker protein stain heavily. Cells that do not stain contain either mutated marker and fail to produce antigenically recognizable marker protein, or are cells in which the pathways that regulate marker levels are dysregulated, resulting in steady state expression of negligible marker protein.

One of skill in the art will recognize that the concentration of a particular antibody used to practice the methods of the invention will vary depending on such factors as time for binding, level of specificity of the antibody for a marker of the invention, and method of sample preparation. Moreover, when multiple antibodies are used, the required concentration may be affected by the order in which the antibodies are applied to the sample, e.g., simultaneously as a cocktail or sequentially as individual antibody reagents. Furthermore, the detection chemistry used to visualize antibody binding to a marker of the invention must also be optimized to produce the desired signal to noise ratio.

In one embodiment of the invention, proteomic methods, e.g., mass spectrometry, are used for detecting and quantitating the marker proteins of the invention. For example, matrix-associated laser desorption/ionization time-of-flight mass spectrometry (MALDI-TOF MS) or surface-enhanced laser desorption/ionization time-of-flight mass spectrometry (SELDI-TOF MS) which involves the application of a biological sample, such as serum, to a protein-binding chip (Wright, G. L., Jr., et al. (2002) Expert Rev Mol Diagn 2:549; Li, J., et al. (2002) Clin Chem 48:1296; Laronga, C., et al. (2003) Dis Markers 19:229; Petricoin, E. F., et al. (2002) 359:572; Adam, B. L., et al. (2002) Cancer Res 62:3609; Tolson, J., et al. (2004) Lab Invest 84:845; Xiao, Z., et al. (2001) Cancer Res 61:6029) can be used to detect and quantitate the PY-Shc and/or p66-Shc proteins. Mass spectrometric methods are described in, for example, U.S. Pat. Nos. 5,622,824, 5,605,798 and 5,547,835, the entire contents of each of which are incorporated herein by reference.

In other embodiments, the expression of a marker of the invention is detected at the nucleic acid level. Nucleic acid-based techniques for assessing expression are well known in the art and include, for example, determining the level of marker mRNA in a sample from a subject. Many expression detection methods use isolated RNA. Any RNA isolation technique that does not select against the isolation of mRNA can be utilized for the purification of RNA from cells that express a marker of the invention (see, e.g., Ausubel et al., ed., (1987-1999) Current Protocols in Molecular Biology (John Wiley & Sons, New York). Additionally, large numbers of tissue samples can readily be processed using techniques well known to those of skill in the art, such as, for example, the single-step RNA isolation process of Chomczynski (1989, U.S. Pat. No. 4,843,155).

The term “probe” refers to any molecule that is capable of selectively binding to a marker of the invention, for example, a nucleotide transcript and/or protein. Probes can be synthesized by one of skill in the art, or derived from appropriate biological preparations. Probes may be specifically designed to be labeled. Examples of molecules that can be utilized as probes include, but are not limited to, RNA, DNA, proteins, antibodies, and organic molecules.

Isolated mRNA can be used in hybridization or amplification assays that include, but are not limited to, Southern or Northern analyses, polymerase chain reaction analyses and probe arrays. One method for the detection of mRNA levels involves contacting the isolated mRNA with a nucleic acid molecule (probe) that can hybridize to the marker mRNA. The nucleic acid probe can be, for example, a full-length cDNA, or a portion thereof, such as an oligonucleotide of at least 7, 15, 30, 50, 100, 250 or 500 nucleotides in length and sufficient to specifically hybridize under stringent conditions to marker genomic DNA.

In one embodiment, the mRNA is immobilized on a solid surface and contacted with a probe, for example by running the isolated mRNA on an agarose gel and transferring the mRNA from the gel to a membrane, such as nitrocellulose. In an alternative embodiment, the probe(s) are immobilized on a solid surface and the mRNA is contacted with the probe(s), for example, in an Affymetrix gene chip array. A skilled artisan can readily adapt known mRNA detection methods for use in detecting the level of marker mRNA.

An alternative method for determining the level of marker mRNA in a sample involves the process of nucleic acid amplification, e.g., by RT-PCR (the experimental embodiment set forth in Mullis, 1987, U.S. Pat. No. 4,683,202), ligase chain reaction (Barany (1991) Proc. Natl. Acad. Sci. USA 88:189-193), self sustained sequence replication (Guatelli et al. (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio/Technology 6:1197), rolling circle replication (Lizardi et al., U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. In particular aspects of the invention, marker expression is assessed by quantitative fluorogenic RT-PCR (i.e., the TaqMan™ System). Such methods typically utilize pairs of oligonucleotide primers that are specific for a marker of the invention. Methods for designing oligonucleotide primers specific for a known sequence are well known in the art.

The expression levels of a marker of the invention may be monitored using a membrane blot (such as used in hybridization analysis such as Northern, Southern, dot, and the like), or microwells, sample tubes, gels, beads or fibers (or any solid support comprising bound nucleic acids). See U.S. Pat. Nos. 5,770,722, 5,874,219, 5,744,305, 5,677,195 and 5,445,934, which are incorporated herein by reference. The detection of marker expression may also comprise using nucleic acid probes in solution.

In one embodiment of the invention, microarrays are used to detect the expression of a marker of the invention. Microarrays are particularly well suited for this purpose because of the reproducibility between different experiments. DNA microarrays provide one method for the simultaneous measurement of the expression levels of large numbers of genes. Each array consists of a reproducible pattern of capture probes attached to a solid support. Labeled RNA or DNA is hybridized to complementary probes on the array and then detected by laser scanning Hybridization intensities for each probe on the array are determined and converted to a quantitative value representing relative gene expression levels. See, U.S. Pat. Nos. 6,040,138, 5,800,992 and 6,020,135, 6,033,860, and 6,344,316, which are incorporated herein by reference. High-density oligonucleotide arrays are particularly useful for determining the gene expression profile for a large number of RNA's in a sample.

The amounts of marker, and/or a mathematical relationship of the amounts of a marker of the invention may be used to calculate the risk of recurrence of a sarcoma in a subject being treated for a sarcoma, the survival of a subject being treated for sarcoma, whether a sarcoma is aggressive, the efficacy of a treatment regimen for treating a sarcoma, and the like, using the methods of the invention, which may include methods of regression analysis known to one of skill in the art. For example, suitable regression models include, but are not limited to CART (e.g., Hill, T, and Lewicki, P. (2006) “STATISTICS Methods and Applications” StatSoft, Tulsa, Okla.), Cox (e.g., www.evidence-based-medicine.co.uk), exponential, normal and log normal (e.g., www.obgyn.cam.ac.uk/mrg/statsbook/stsurvan.html), logistic (e.g., www.en.wikipedia.org/wiki/Logistic_regression), parametric, non-parametric, semi-parametric (e.g., www.socserv.mcmaster.ca/j fox/Books/Companion), linear (e.g., www.en.wikipedia.org/wiki/Linear_regression), or additive (e.g., www.en.wikipedia.org/wiki/Generalized_additive_model).

In one embodiment, a regression analysis includes the amounts of marker. In another embodiment, a regression analysis includes a marker mathematical relationship. In yet another embodiment, a regression analysis of the amounts of marker, and/or a marker mathematical relationship may include additional clinical and/or molecular co-variates. Such clinical co-variates include, but are not limited to, nodal status, tumor stage, tumor grade, tumor size, treatment regime, e.g., chemotherapy and/or radiation therapy, clinical outcome (e.g., relapse, disease-specific survival, therapy failure), and/or clinical outcome as a function of time after diagnosis, time after initiation of therapy, and/or time after completion of treatment.

In another embodiment, the amounts of marker, and/or a mathematical relationship of the amounts of a marker may be used to calculate the risk of recurrence of a sarcoma in a subject being treated for a sarcoma, the survival of a subject being treated for a sarcoma, whether a sarcoma is aggressive, the efficacy of a treatment regimen for treating a sarcoma, and the like, using the methods of the invention, which may include methods of regression analysis known to one of skill in the art. For example, suitable regression models include, but are not limited to CART (e.g., Hill, T, and Lewicki, P. (2006) “STATISTICS Methods and Applications” StatSoft, Tulsa, Okla.), Cox (e.g., www.evidence-based-medicine.co.uk), exponential, normal and log normal (e.g., www.obgyn.cam.ac.uk/mrg/statsbook/stsurvan.html), logistic (e.g., www.en.wikipedia.org/wiki/Logistic_regression), parametric, non-parametric, semi-parametric (e.g., www.socserv.mcmaster.ca/jfox/Books/Companion), linear (e.g., www.en.wikipedia.org/wiki/Linear_regression), or additive (e.g., www.en.wikipedia.org/wiki/Generalized_additive_model).

In one embodiment, a regression analysis includes the amounts of marker. In another embodiment, a regression analysis includes a marker mathematical relationship. In yet another embodiment, a regression analysis of the amounts of marker, and/or a marker mathematical relationship may include additional clinical and/or molecular co-variates. Such clinical co-variates include, but are not limited to, nodal status, tumor stage, tumor grade, tumor size, treatment regime, e.g., chemotherapy and/or radiation therapy, clinical outcome (e.g., relapse, disease-specific survival, therapy failure), and/or clinical outcome as a function of time after diagnosis, time after initiation of therapy, and/or time after completion of treatment.

### VIII. Kits

The invention also provides compositions and kits for prognosing a sarcoma, recurrence of a sarcoma, or survival of a subject being treated for a sarcoma. These kits include one or more of the following: a detectable antibody that specifically binds to a marker of the invention, a detectable antibody that specifically binds to a marker of the invention, reagents for obtaining and/or preparing subject tissue samples for staining, and instructions for use.

The kits of the invention may optionally comprise additional components useful for performing the methods of the invention. By way of example, the kits may comprise fluids (e.g., SSC buffer) suitable for annealing complementary nucleic acids or for binding an antibody with a protein with which it specifically binds, one or more sample compartments, an instructional material which describes performance of a method of the invention and tissue specific controls/standards.

### IX. Screening Assays

Targets of the invention include, but are not limited to, the genes subsequently listed in Tables 2-9 herein. Based on the results of experiments described by Applicants herein, the key proteins modulated by Q10 are associated with or can be classified into different pathways or groups of molecules, including cytoskeletal components, transcription factors, apoptotic response, pentose phosphate pathway, biosynthetic pathway, oxidative stress (pro-oxidant), membrane alterations, and oxidative phosphorylation metabolism.

Accordingly, in one embodiment of the invention, a marker may include ANGPTL3, CCL2, CDH5, CXCL1, CXCL3, PRMT3, HDAC2, Nitric Oxide Synthase bNOS, Acetyl phospho Histone H3 AL9 S10, MTA 2, Glutamic Acid Decarboxylase GAD65 67, KSR, HDAC4, BOB1 OBF1, a1Syntrophin, BAP1, Importina 57, α E-Catenin, Grb2, Bax, Proteasome 26S subunit 13 (Endophilin B1), Actin-like 6A (Eukaryotic Initiation Factor 4A11), Nuclear Chloride Channel protein, Proteasome 26S subunit, Dismutase Cu/Zn Superoxide, Translin-associated factor X, Arsenite translocating ATPase (Spermine synthetase), ribosomal protein SA, dCTP pyrophosphatase 1, proteasome beta 3, proteasome beta 4, acid phosphatase 1, diazepam binding inhibitor, alpha 2-HS glycoprotein (Bos Taurus, cow), ribosomal protein P2 (RPLP2); histone H2A, microtubule associated protein, proteasome alpha 3, eukaryotic translation elongation factor 1 delta, lamin B1, SMT 3 suppressor of mif two 3 homolog 2, heat shock protein 27 kD, hnRNP C1/C2, eukaryotc translation elongation factor 1 beta 2, Similar to HSPC-300, DNA directed DNA polymerase epislon 3; (canopy 2 homolog), LAMA5, PXLDC1, p300 CBP, P53R2, Phosphatidylserine Receptor, Cytokeratin Peptide 17, Cytokeratin peptide 13, Neurofilament 160 200, Rab5, Filensin, P53R2, MDM2, MSH6, Heat Shock Factor 2, AFX, FLIPg d, JAB 1, Myosine, MEKK4, cRaf pSer621, FKHR FOXO1a, MDM2, Fas Ligand, P53R2, Myosin Regulatory Light Chain, hnRNP C1/C2, Ubiquilin 1 (Phosphatase 2A), hnRNP C1/C2, alpha 2-HS glycoprotein (Bos Taurus, cow), beta actin, hnRNP C1/C2, heat shock protein 70 kD, beta tubulin, ATP dependent helicase II, eukaryotc translation elongation factor 1 beta 2, ER lipid raft associated 2 isoform 1 (beta actin), signal sequence receptor 1 delta, Eukaryotic translation initiation factor 3, subunit 3 gamma, Bilverdin reductase A (Transaldolase 1), Keratin 1,10 (Parathymosin), GST omega 1, chain B Dopamine Quinone Conjugation to Dj-1, Proteasome Activator Reg (alpha), T-complex protein 1 isoform A, Chain A Tapasin ERP57 (Chaperonin containing TCP1), Ubiquitin activating enzyme E1; Alanyl-tRNA synthetase, Dynactin 1, Heat shock protein 60 kd, Beta Actin, Spermidine synthase (Beta Actin), Heat Shock protein 70 kd, retinoblastoma binding protein 4 isoform A, TAR DNA binding protein, eukaryotic translation elongation factor 1 beta 2, chaperonin containing TCP1, subunit 3, cytoplasmic dynein IC-2, Angiotensin-converting enzyme (ACE), Caspase 3, GARS, Matrix Metalloproteinase 6 (MMP-6), Neurolysin (NLN)-Catalytic Domain, and Neurolysin (NLN), ADRB, CEACAM1, DUSP4, FOXC2, FOXP3, GCGR, GPD1, HMOX1, IL4R, INPPL1, IRS2 and VEGFA.

Screening assays useful for identifying modulators of identified markers are described below.

The invention also provides methods (also referred to herein as “screening assays”) for identifying modulators, i.e., candidate or test compounds or agents (e.g., proteins, peptides, peptidomimetics, peptoids, small molecules or other drugs), which are useful for treating or preventing a sarcoma by modulating the expression and/or activity of a marker of the invention. Such assays typically comprise a reaction between a marker of the invention and one or more assay components. The other components may be either the test compound itself, or a combination of test compounds and a natural binding partner of a marker of the invention. Compounds identified via assays such as those described herein may be useful, for example, for modulating, e.g., inhibiting, ameliorating, treating, or preventing aggressiveness of a sarcoma.

The test compounds used in the screening assays of the present invention may be obtained from any available source, including systematic libraries of natural and/or synthetic compounds. Test compounds may also be obtained by any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive; see, e.g., Zuckermann et al., 1994, J. Med. Chem. 37:2678-85); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam, 1997, Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented in solution (e.g., Houghten, 1992, Biotechniques 13:412-421), or on beads (Lam, 1991, Nature 354:82-84), chips (Fodor, 1993, Nature 364:555-556), bacteria and/or spores, (Ladner, U.S. Pat. No. 5,223,409), plasmids (Cull et al, 1992, Proc Natl Acad Sci USA 89:1865-1869) or on phage (Scott and Smith, 1990, Science 249:386-390; Devlin, 1990, Science 249:404-406; Cwirla et al, 1990, Proc. Natl. Acad. Sci. 87:6378-6382; Felici, 1991, J. Mol. Biol. 222:301-310; Ladner, supra.).

The screening methods of the invention comprise contacting a sarcoma cell with a test compound and determining the ability of the test compound to modulate the expression and/or activity of a marker of the invention in the cell. The expression and/or activity of a marker of the invention can be determined as described herein.

In another embodiment, the invention provides assays for screening candidate or test compounds which are substrates of a marker of the invention or biologically active portions thereof. In yet another embodiment, the invention provides assays for screening candidate or test compounds which bind to a marker of the invention or biologically active portions thereof. Determining the ability of the test compound to directly bind to a marker can be accomplished, for example, by coupling the compound with a radioisotope or enzymatic label such that binding of the compound to the marker can be determined by detecting the labeled marker compound in a complex. For example, compounds (e.g., marker substrates) can be labeled with ¹³¹I, ¹²⁵I, ³⁵S, ¹⁴C, or ³H, either directly or indirectly, and the radioisotope detected by direct counting of radioemission or by scintillation counting. Alternatively, assay components can be enzymatically labeled with, for example, horseradish peroxidase, alkaline phosphatase, or luciferase, and the enzymatic label detected by determination of conversion of an appropriate substrate to product.

This invention further pertains to novel agents identified by the above-described screening assays. Accordingly, it is within the scope of this invention to further use an agent identified as described herein in an appropriate animal model. For example, an agent capable of modulating the expression and/or activity of a marker of the invention identified as described herein can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatment as described above.

### X. Pharmaceutical Compositions and Pharmaceutical Administration

The present invention provides compositions comprising a CoQ10 molecule, e.g., CoQ10. A CoQ10 molecule can be incorporated into pharmaceutical compositions suitable for administration to a subject. Typically, the pharmaceutical composition comprises a CoQ10 molecule and a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the environmental influencer.

The compositions of this invention may be in a variety of forms. These include, for example, liquid, semi-solid and solid dosage forms, such as liquid solutions (e.g., injectable and infusible solutions), dispersions or suspensions, tablets, pills, powders, creams, lotions, liniments, ointments or pastes, drops for administration to the eye, ear or nose, liposomes and suppositories. The preferred form depends on the intended mode of administration and therapeutic application.

CoQ10 molecules can be administered by a variety of methods known in the art. For many therapeutic applications, the preferred route/mode of administration is topical, subcutaneous injection, intravenous injection or infusion. As will be appreciated by the skilled artisan, the route and/or mode of administration will vary depending upon the desired results. In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978. In one embodiment, the mode of administration is parenteral (e.g., intravenous, subcutaneous, intraperitoneal, intramuscular). In one embodiment, the environmental influencer is administered by intravenous infusion or injection. In another embodiment, the environmental influencer is administered by intramuscular or subcutaneous injection. In a preferred embodiment, the environmental influencer is administered topically.

Therapeutic compositions typically must be sterile and stable under the conditions of manufacture and storage. The composition can be formulated as a solution, microemulsion, dispersion, liposome, or other ordered structure suitable to high drug concentration. Sterile injectable solutions can be prepared by incorporating the active compound (i.e., environmental influencer) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile, lyophilized powders for the preparation of sterile injectable solutions, the preferred methods of preparation are vacuum drying and spray-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

Techniques and formulations generally may be found in Remmington's Pharmaceutical Sciences, Meade Publishing Co., Easton, Pa. For systemic administration, injection is preferred, including intramuscular, intravenous, intraperitoneal, and subcutaneous. For injection, the compounds of the invention can be formulated in liquid solutions, preferably in physiologically compatible buffers such as Hank's solution or Ringer's solution. In addition, the compounds may be formulated in solid form and redissolved or suspended immediately prior to use. Lyophilized forms are also included.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., ationd oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound. For buccal administration the compositions may take the form of tablets or lozenges formulated in conventional manner. For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration bile salts and fusidic acid derivatives in addition, detergents may be used to facilitate permeation. Transmucosal administration may be through nasal sprays or using suppositories. For topical administration, the compound(s) of the invention are formulated into ointments, salves, gels, or creams as generally known in the art. A wash solution can be used locally to treat an injury or inflammation to accelerate healing.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

For therapies involving the administration of nucleic acids, the compound(s) of the invention can be formulated for a variety of modes of administration, including systemic and topical or localized administration. Techniques and formulations generally may be found in Remmington's Pharmaceutical Sciences, Meade Publishing Co., Easton, Pa. For systemic administration, injection is preferred, including intramuscular, intravenous, intraperitoneal, intranodal, and subcutaneous. For injection, the compound(s) of the invention can be formulated in liquid solutions, preferably in physiologically compatible buffers such as Hank's solution or Ringer's solution. In addition, the compound(s) may be formulated in solid form and redissolved or suspended immediately prior to use. Lyophilized forms are also included.

In a preferred embodiment of the invention, the compositions comprising a CoQ10 molecule, e.g., CoQ10, are administered topically. It is preferable to present the active ingredient, i.e. a CoQ10 molecule, as a pharmaceutical formulation. The active ingredient may comprise, for topical administration, from about 0.001% to about 20% w/w, by weight of the formulation in the final product, although it may comprise as much as 30% w/w, preferably from about 1% to about 20% w/w of the formulation. The topical formulations of the present invention, comprise an active ingredient together with one or more acceptable carrier(s) therefor and optionally any other therapeutic ingredients(s). The carrier(s) should be “acceptable” in the sense of being compatible with the other ingredients of the formulation and not deleterious to the recipient thereof.

In treating a patient exhibiting a disorder of interest, a therapeutically effective amount of an agent or agents such as these is administered. A therapeutically effective dose refers to that amount of the compound that results in amelioration of symptoms or a prolongation of survival in a patient.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic indices are preferred. The data obtained from these cell culture assays and animal studies can be used in formulating a range of dosage for use in human. The dosage of such compounds lies preferably within a range of circulating concentrations that include the ED₅₀ with little or no toxicity. The dosage may vary within this range depending upon the dosage form employed and the route of administration utilized.

For any compound used in the method of the invention, the therapeutically effective dose can be estimated initially from cell culture assays. For example, a dose can be formulated in animal models to achieve a circulating plasma concentration range that includes the IC₅₀ as determined in cell culture. Such information can be used to more accurately determine useful doses in humans. Levels in plasma may be measured, for example, by HPLC.

The exact formulation, route of administration and dosage can be chosen by the individual physician in view of the patient's condition. (See e.g. Fingl et al., in The Pharmacological Basis of Therapeutics, 1975, Ch. 1 p. 1). It should be noted that the attending physician would know how to and when to terminate, interrupt, or adjust administration due to toxicity, or to organ dysfunctions. Conversely, the attending physician would also know to adjust treatment to higher levels if the clinical response were not adequate (precluding toxicity). The magnitude of an administrated dose in the management of the oneogenic disorder of interest will vary with the severity of the condition to be treated and to the route of administration. The severity of the condition may, for example, be evaluated, in part, by standard prognostic evaluation methods. Further, the dose and perhaps dose frequency, will also vary according to the age, body weight, and response of the individual patient. A program comparable to that discussed above may be used in veterinary medicine.

Depending on the specific conditions being treated, such agents may be formulated and administered systemically or locally. Techniques for formulation and administration may be found in Remington's Pharmaceutical Sciences, 18^{th} ed., Mack Publishing Co., Easton, Pa. (1990). Suitable routes may include oral, rectal, transdermal, vaginal, transmucosal, or intestinal administration; parenteral delivery, including intramuscular, subcutaneous, intramedullary injections, as well as intrathecal, direct intraventricular, intravenous, intraperitoneal, intranasal, or intraocular injections, just to name a few.

The compositions described above may be administered to a subject in any suitable formulation. In addition to treatment of a sarcoma with topical formulations of a CoQ10 molecule, e.g., CoQ10, in other aspects of the invention a CoQ10 molecule might be delivered by other methods. For example, a CoQ10 molecule might be formulated for parenteral delivery, e.g., for subcutaneous, intravenous, intramuscular, or intratumoral injection. Other methods of delivery, for example, liposomal delivery or diffusion from a device impregnated with the composition might be used. The compositions may be administered in a single bolus, multiple injections, or by continuous infusion (for example, intravenously or by peritoneal dialysis). For parenteral administration, the compositions are preferably formulated in a sterilized pyrogen-free form. Compositions of the invention can also be administered in vitro to a cell (for example, to induce apoptosis in a cancer cell in an in vitro culture) by simply adding the composition to the fluid in which the cell is contained.

For injection, the agents of the invention may be formulated in aqueous solutions, preferably in physiologically compatible buffers such as Hanks's solution, Ringer's solution, or physiological saline buffer. For such transmucosal administration, penetrants appropriate to the barrier to be permeated are used in the formulation. Such penetrants are generally known in the art.

Use of pharmaceutically acceptable carriers to formulate the compounds herein disclosed for the practice of the invention into dosages suitable for systemic administration is within the scope of the invention. With proper choice of carrier and suitable manufacturing practice, the compositions of the present invention, in particular, those formulated as solutions, may be administered parenterally, such as by intravenous injection. The compounds can be formulated readily using pharmaceutically acceptable carriers well known in the art into dosages suitable for oral administration. Such carriers enable the compounds of the invention to be formulated as tablets, pills, capsules, liquids, gels, syrups, slurries, suspensions. and the like, for oral ingestion by a patient to be treated.

Agents intended to be administered intracellularly may be administered using techniques well known to those of ordinary skill in the art. For example, such agents may be encapsulated into liposomes, then administered as described above. Liposomes are spherical lipid bilayers with aqueous interiors. All molecules present in an aqueous solution at the time of liposome formation are incorporated into the aqueous interior. The liposomal contents are both protected from the external microenvironment and, because liposomes fuse with cell membranes, are efficiently delivered into the cell cytoplasm. Additionally, due to their hydrophobicity, small organic molecules may be directly administered intracellularly.

Pharmaceutical compositions suitable for use in the present invention include compositions wherein the active ingredients are contained in an effective amount to achieve its intended purpose. Determination of the effective amounts is well within the capability of those skilled in the art, especially in light of the detailed disclosure provided herein. In addition to the active ingredients, these pharmaceutical compositions may contain suitable pharmaceutically acceptable carriers comprising excipients and auxiliaries which facilitate processing of the active compounds into preparations which can be used pharmaceutically. The preparations formulated for oral administration may be in the form of tablets, dragees, capsules, or solutions. The pharmaceutical compositions of the present invention may be manufactured in a manner that is itself known, e.g., by means of conventional mixing, dissolving, granulating, dragee-making, levitating, emulsifying, encapsulating, entrapping or lyophilizing processes. Formulations suitable for topical administration include liquid or semi-liquid preparations suitable for penetration through the skin to the site of where treatment is required, such as liniments, lotions, creams, ointments or pastes, and drops suitable for administration to the eye, ear, or nose. Drops according to the present invention may comprise sterile aqueous or oily solutions or suspensions and may be prepared by dissolving the active ingredient in a suitable aqueous solution of a bactericidal and/or fungicidal agent and/or any other suitable preservative, and preferably including a surface active agent. The resulting solution may then be clarified and sterilized by filtration and transferred to the container by an aseptic technique. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

Lotions according to the present invention include those suitable for application to the skin or eye. An eye lotion may comprise a sterile aqueous solution optionally containing a bactericide and may be prepared by methods similar to those for the preparation of drops. Lotions or liniments for application to the skin may also include an agent to hasten drying and to cool the skin, such as an alcohol or acetone, and/or a moisturizer such as glycerol or an oil such as castor oil or arachis oil.

Creams, ointments or pastes according to the present invention are semi-solid formulations of the active ingredient for external application. They may be made by mixing the active ingredient in finely-divided or powdered form, alone or in solution or suspension in an aqueous or non-aqueous fluid, with the aid of suitable machinery, with a greasy or non-greasy basis. The basis may comprise hydrocarbons such as hard, soft or liquid paraffin, glycerol, beeswax, a metallic soap; a mucilage; an oil of natural origin such as almond, corn, arachis, castor or olive oil; wool fat or its derivatives, or a fatty acid such as stearic or oleic acid together with an alcohol such as propylene glycol or macrogels. The formulation may incorporate any suitable surface active agent such as an anionic, cationic or non-ionic surface active such as sorbitan esters or polyoxyethylene derivatives thereof. Suspending agents such as natural gums, cellulose derivatives or inorganic materials such as silicaceous silicas, and other ingredients such as lanolin, may also be included.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Pharmaceutical preparations for oral use can be obtained by combining the active compounds with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxy-methylcellulose, and/or polyvinyl pyrrolidone (PVP). If desired, disintegrating agents may be added, such as the cross-linked polyvinyl pyrrolidone, agar, or alginic acid or a salt thereof such as sodium alginate.

Dragee cores are provided with suitable coating. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added.

The composition can include a buffer system, if desired. Buffer systems are chosen to maintain or buffer the pH of compositions within a desired range. The term “buffer system” or “buffer” as used herein refers to a solute agent or agents which, when in a water solution, stabilize such solution against a major change in pH (or hydrogen ion concentration or activity) when acids or bases are added thereto. Solute agent or agents which are thus responsible for a resistance or change in pH from a starting buffered pH value in the range indicated above are well known. While there are countless suitable buffers, potassium phosphate monohydrate is a preferred buffer.

The final pH value of the pharmaceutical composition may vary within the physiological compatible range. Necessarily, the final pH value is one not irritating to human skin and preferably such that transdermal transport of the active compound, i.e. a CoQ10 molecule is facilitated. Without violating this constraint, the pH may be selected to improve a CoQ10 molecule stability and to adjust consistency when required. In one embodiment, the preferred pH value is about 3.0 to about 7.4, more preferably about 3.0 to about 6.5, most preferably from about 3.5 to about 6.0.

For preferred topical delivery vehicles the remaining component of the composition is water, which is necessarily purified, e.g., deionized water. Such delivery vehicle compositions contain water in the range of more than about 50 to about 95 percent, based on the total weight of the composition. The specific amount of water present is not critical, however, being adjustable to obtain the desired viscosity (usually about 50 cps to about 10,000 cps) and/or concentration of the other components. The topical delivery vehicle preferably has a viscosity of at least about 30 centipoises.

Other known transdermal skin penetration enhancers can also be used to facilitate delivery of a CoQ10 molecule. Illustrative are sulfoxides such as dimethylsulfoxide (DMSO) and the like; cyclic amides such as 1-dodecylazacycloheptane-2-one (Azone™, a registered trademark of Nelson Research, Inc.) and the like; amides such as N,N-dimethyl acetamide (DMA) N,N-diethyl toluamide, N,N-dimethyl formamide, N,N-dimethyl octamide, N,N-dimethyl decamide, and the like; pyrrolidone derivatives such as N-methyl-2-pyrrolidone, 2-pyrrolidone, 2-pyrrolidone-5-carboxylic acid, N-(2-hydroxyethyl)-2-pyrrolidone or fatty acid esters thereof, 1-lauryl-4-methoxycarbonyl-2-pyrrolidone, N-tallowalkylpyrrolidones, and the like; polyols such as propylene glycol, ethylene glycol, polyethylene glycol, dipropylene glycol, glycerol, hexanetriol, and the like; linear and branched fatty acids such as oleic, linoleic, lauric, valeric, heptanoic, caproic, myristic, isovaleric, neopentanoic, trimethyl hexanoic, isostearic, and the like; alcohols such as ethanol, propanol, butanol, octanol, oleyl, stearyl, linoleyl, and the like; anionic surfactants such as sodium laurate, sodium lauryl sulfate, and the like; cationic surfactants such as benzalkonium chloride, dodecyltrimethylammonium chloride, cetyltrimethylammonium bromide, and the like; non-ionic surfactants such as the propoxylated polyoxyethylene ethers, e.g., Poloxamer 231, Poloxamer 182, Poloxamer 184, and the like, the ethoxylated fatty acids, e.g., Tween 20, Myjr 45, and the like, the sorbitan derivatives, e.g., Tween 40, Tween 60, Tween 80, Span 60, and the like, the ethoxylated alcohols, e.g., polyoxyethylene (4) lauryl ether (Brij 30), polyoxyethylene (2) oleyl ether (Brij 93), and the like, lecithin and lecithin derivatives, and the like; the terpenes such as D-limonene, .alpha.-pinene, .beta.-carene, .alpha.-terpineol, carvol, carvone, menthone, limonene oxide, .alpha.-pinene oxide, eucalyptus oil, and the like. Also suitable as skin penetration enhancers are organic acids and esters such as salicyclic acid, methyl salicylate, citric acid, succinic acid, and the like.

In one embodiment, the present invention provides CoQ10 molecule compositions and methods of preparing the same. Preferably, the compositions comprise at least about 1% to about 25% of a CoQ10 molecule, e.g., CoQ10, w/w. CoQ10 can be obtained from Asahi Kasei N&P (Hokkaido, Japan) as UBIDECARENONE (USP). CoQ10 can also be obtained from Kaneka Q10 as Kaneka Q10 (USP UBIDECARENONE) in powdered form (Pasadena, Tex., USA). CoQ10 used in the methods exemplified herein have the following characteristics: residual solvents meet USP 467 requirement; water content is less than 0.0%, less than 0.05% or less than 0.2%; residue on ignition is 0.0%, less than 0.05%, or less than 0.2% less than; heavy metal content is less than 0.002%, or less than 0.001%; purity of between 98-100% or 99.9%, or 99.5%. Methods of preparing the compositions are provided in the examples section below.

In certain embodiments of the invention, methods are provided for treating or preventing sarcoma in a human by topically administering a Coenzyme Q10 molecule, e.g., CoQ10, to the human such that treatment or prevention occurs, wherein the human is administered a topical dose of a Coenzyme Q10 molecule, e.g., CoQ10, in a topical vehicle where the Coenzyme Q10 molecule is applied to the target tissue in the range of about 0.01 to about 0.5 milligrams of the coenzyme Q10 molecule, e.g., CoQ10, per square centimeter of skin. In one embodiment, the Coenzyme Q10 molecule, e.g., CoQ10, is applied to the target tissue in the range of about 0.09 to about 0.15 mg CoQ10 per square centimeter of skin. In various embodiments, the Coenzyme Q10 molecule, e.g., CoQ10, is applied to the target tissue in the range of about 0.001 to about 5.0, about 0.005 to about 1.0, about 0.005 to about 0.5, about 0.01 to about 0.5, about 0.025 to about 0.5, about 0.05 to about 0.4, about 0.05 to about 0.30, about 0.10 to about 0.25, or about 0.10 to 0.20 mg CoQ10 molecule, e.g., CoQ10, per square centimeter of skin. In other embodiments, the Coenzyme Q10 molecule, e.g., CoQ10, is applied to the target tissue at a dose of about 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49 or 0.5 mg CoQ10 per square centimeter of skin. In one embodiment, the Coenzyme Q10 molecule, e.g, CoQ10, is applied to the target tissue at a dose of about 0.12 mg of the CoQ10 molecule, e.g., CoQ10, per square centimeter of skin. It should be understood that ranges having any one of these values as the upper or lower limits are also intended to be part of this invention, e.g., about 0.03 to about 0.12, about 0.05 to about 0.15, about 0.1 to about 0.20, or about 0.32 to about 0.49 mg per square centimeter of skin.

In another embodiment of the invention, the Coenzyme Q10 molecule is administered in the form of a CoQ10 molecule cream at a dosage of between 0.5 and 10 milligrams of the CoQ10 molecule cream per square centimeter of skin, wherein the CoQ10 molecule cream comprises between 1 and 5% of the Coenzyme Q10 molecule, e.g., CoQ10. In one embodiment, the CoQ10 molecule, e.g., CoQ10, cream comprises about 3% of the Coenzyme Q10 molecule, e.g., CoQ10. In other embodiments, the CoQ10 molecule cream comprises about 1%, 1.5%, 2%, 2.5%, 3%, 3.5%, 4%, 4.5% or 5% of the Coenzyme Q10 molecule, e.g., CoQ10. In various embodiments, the CoQ10 molecule cream is administered at a dosage of about 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5 or 10 milligrams of CoQ10 molecule, e.g., CoQ10 cream per square centimeter of skin. It should be understood that ranges having any one of these values as the upper or lower limits are also intended to be part of this invention, e.g., between about 0.5 and about 5.0, about 1.5 and 2.5, or about 2.5 and 5.5 mg CoQ10 molecule, e.g., CoQ10, cream per square centimeter of skin.

In another embodiment, the Coenzyme Q10 molecule is administered in the form of a CoQ10 cream at a dosage of between 3 and 5 milligrams of the CoQ10 molecule, e.g., CoQ10, cream per square centimeter of skin, wherein the CoQ10 molecule, e.g., CoQ10, cream comprises between 1 and 5% of Coenzyme Q10. In one embodiment, the CoQ10 molecule, e.g., CoQ10, cream comprises about 3% of Coenzyme Q10. In other embodiments, the CoQ10 molecule, e.g., CoQ10, cream comprises about 1%, 1.5%, 2%, 2.5%, 3%, 3.5%, 4%, 4.5% or 5% of Coenzyme Q10. In various embodiments, the CoQ10 molecule, e.g., CoQ10, cream is administered at a dosage of about 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9 or 5.0 milligrams of CoQ10 molecule, e.g., CoQ10, cream per square centimeter of skin. It should be understood that ranges having any one of these values as the upper or lower limits are also intended to be part of this invention, e.g., between about 3.0 and about 4.0, about 3.3 and 5.3, or about 4.5 and 4.9 mg CoQ10 molecule, e.g., CoQ10, cream per square centimeter of skin.

Certain aspects of the invention provide methods for treating or preventing sarcoma in a human by topically administering Coenzyme Q10 to the human such that treatment or prevention occurs, wherein the Coenzyme Q10 is topically applied one or more times per 24 hours for six weeks or more.

Certain aspects of the invention provide methods for the preparation of a Coenzyme Q10 cream 3% which includes the steps of preparing a Phase A, B, C, D and E and combining all the phases such that an oil-in-water emulsion of 3% CoQ10 cream is formed.

In some embodiments, the Phase A ingredients include Alkyl C₁₂₋₁₅ benzoate NF at 4.00% w/w, cetyl alcohol NF at 2.00% w/w, glyceryl stearate/PEG-100 at 4.5% w/w and stearyl alcohol NF at 1.50% w/w while the Phase B ingredients include diethylene glycol monoethyl ether NF at 5.00% w/w, glycerin USP at 2.00% w/w, propylene glycol USP at 1.50% w/w, phenoxyethanol NF at 0.475% w/w, purified water USP at 16.725% w/w and Carbomer Dispersion 2% at 40.00% w/w and the Phace C ingredients include lactic acid USP at 0.50% w/w, sodium lactate solution USP at 2.00% w/w, trolamine NF at 1.30% w/w, and purified water USP at 2.50% w/w. Furthermore in these embodiments the Phase D ingredients include titanium dioxide USP at 1.00% w/w while the Phase E ingredients include CoQ10 21% concentrate at 15% w/w.

In certain other embodiments, the Phase A ingredients include capric/caprylic triglyceride at 4.00% w/w, cetyl alcohol NF at 2.00% w/w, glyceril stearate/PEG-100 at 4.5% and stearyl alcohol NF at 1.5% w/w while the Phase B ingredients include diethylene glycol monoethyl ether NF at 5.00% w/w, glycerin USP at 2.00% w/w, propylene glycol USP at 1.50% w/w, phenoxyethanol NF at 0.475% w/w, purified water USP at 16.725% w/w and Carbomer Dispersion 2% at 40.00% w/w and the Phace C ingredients include lactic acid USP at 0.50% w/w, sodium lactate solution USP at 2.00% w/w, trolamine NF at 1.30% w/w, and purified water USP at 2.50% w/w. Furthermore in these embodiments the Phase D ingredients include titanium dioxide USP at 1.00% w/w while the Phase E ingredients include CoQ10 21% concentrate at 15% w/w.

In certain embodiments of the invention, methods are provided for the preparation of a Coenzyme Q10 cream 3% which include the steps of (1) adding the Phase A ingredients to a suitable container and heating to 70-80 degrees C. in a water bath; (2) adding the Phase B ingredients, excluding the Carbomer Dispersion, to a suitable container and mixing to form a mixed Phase B; (3) placing the Phase E ingredients into a suitable container and melting them at 50-60 degrees C. using a water bath to form a melted Phase E; (4) adding the Carbomer Dispersion to a Mix Tank and heating to 70-80 degrees C. while mixing; (5) adding the mixed Phase B to the Mix Tank while maintaining the temperature at 70-80 degrees C.; (6) adding the Phase C ingredients to the Mix Tank while maintaining the temperature at 70-80 degrees C.; (7) adding the Phase D ingredients to the Mix Tank and then continue mixing and homogenizing the contents of the Mix Tank; then (8) stopping the homogenization and cooling the contents of the Mix Tank to 50-60 degrees C.; then (9) discontinuing the mixing and adding the melted Phase E to the Mix Tank to form a dispersion; (10) mixing is then resumed until the dispersion is smooth and uniform; then (11) cooling the contents of the Mix Tank to 45-50 degrees C.

In some other embodiments of the invention, a pharmaceutical composition comprising CoQ10 cream 3% is provided. The cream includes a phase A having C₁₂₋₁₅ alkyl benzoate at 4.00% w/w of the composition, cetyl alcohol at 2.00% w/w of the composition, stearyl alcohol at 1.5% w/w, glyceryl stearate and PEG-100 at 4.5% w/w; a phase B having glycerin at 2.00% w/w, propylene glycol at 1.5% w/w, ethoxydiglycol at 5.0% w/w, phenoxyethanol at 0.475% w/w, a carbomer dispersion at 40.00% w/w, purified water at 16.725% w/w; a phase C having triethanolamine at 1.300% w/w, lactic acid at 0.500% w/w, sodium lactate solution at 2.000% w/w, water at 2.5% w/w; a phase D having titanium dioxide at 1.000% w/w; and a phase E having CoQ10 21% concentrate at 15.000% w/w. In some embodiments the Carbomer Dispersion includes water, phenoxyethanol, propylene glycol and Carbomer 940.

In some other embodiments of the invention, a pharmaceutical composition comprising CoQ10 cream 3% is provided. The cream includes a phase A having Capric/Caprylic triglyceride at 4.00% w/w of the composition, cetyl alcohol at 2.00% w/w of the composition, stearyl alcohol at 1.5% w/w, glyceryl stearate and PEG-100 at 4.5% w/w; a phase B having glycerin at 2.00% w/w, propylene glycol at 1.5% w/w, ethoxydiglycol at 5.0% w/w, phenoxyethanol at 0.475% w/w, a carbomer dispersion at 40.00% w/w, purified water at 16.725% w/w; a phase C having triethanolamine at 1.300% w/w, lactic acid at 0.500% w/w, sodium lactate solution at 2.000% w/w, water at 2.5% w/w; a phase D having titanium dioxide at 1.000% w/w; and a phase E having CoQ10 21% concentrate at 15.000% w/w. In some embodiments the Carbomer Dispersion includes water, phenoxyethanol, propylene glycol and Carbomer 940.

In some other embodiments of the invention, a pharmaceutical composition comprising CoQ10 cream 1.5% is provided. The cream includes a phase A having C₁₂₋₁₅ alkyl benzoate at 5.000% w/w, cetyl alcohol at 2.000% w/w, stearyl alcohol at 1.5% w/w, glyceryl stearate and PEG-100 stearate at 4.500% w/w; a phase B having glycerin at 2.000% w/w, propylene at 1.750% w/w, ethoxydiglycol at 5.000% w/w, phenoxyethanol at 0.463% w/w, a carbomer dispersion at 50% w/w, and purified water at 11.377% w/w; a phase C having triethanolamine at 1.3% w/w, lactic acid at 0.400% w/w, sodium lactate solution at 2.000% w/w, and water at 4.210% w/w; a phase D having titanium dioxide at 1.000% w/w; and a phase E having CoQ10 21% concentrate at 1.500% w/w.

In some other embodiments of the invention, a pharmaceutical composition comprising CoQ10 cream 1.5% is provided. The cream includes a phase A having Capric/Caprylic triglyceride at 5.000% w/w, cetyl alcohol at 2.000% w/w, stearyl alcohol at 1.5% w/w, glyceryl stearate and PEG-100 stearate at 4.500% w/w; a phase B having glycerin at 2.000% w/w, propylene at 1.750% w/w, ethoxydiglycol at 5.000% w/w, phenoxyethanol at 0.463% w/w, a carbomer dispersion at 50% w/w, and purified water at 11.377% w/w; a phase C having triethanolamine at 1.3% w/w, lactic acid at 0.400% w/w, sodium lactate solution at 2.000% w/w, and water at 4.210% w/w; a phase D having titanium dioxide at 1.000% w/w; and a phase E having CoQ10 21% concentrate at 1.500% w/w. In some embodiments the Carbomer Dispersion includes water, phenoxyethanol and propylene glycol.

1. Combination Therapies

In certain embodiments, a CoQ10 molecule and/or pharmaceutical compositions thereof can be used in combination therapy with at least one other therapeutic agent. A CoQ10 molecule and/or pharmaceutical composition thereof and the other therapeutic agent can act additively or, more preferably, synergistically. In one embodiment, A CoQ10 molecule and/or a pharmaceutical composition thereof is administered concurrently with the administration of another therapeutic agent. In another embodiment, a compound and/or pharmaceutical composition thereof is administered prior or subsequent to administration of another therapeutic agent.

In one embodiment, the therapeutic methods of the invention comprise additional agents. For example, in one embodiment, an additional agent for use in the therapeutic methods of the invention of the invention is a chemotherapeutic agent.

Chemotherapeutic agents generally belong to various classes including, for example: 1. Topoisomerase II inhibitors (cytotoxic antibiotics), such as the antracyclines/anthracenediones, e.g., doxorubicin, epirubicin, idarubicin and nemorubicin, the anthraquinones, e.g., mitoxantrone and losoxantrone, and the podophillotoxines, e.g., etoposide and teniposide; 2. Agents that affect microtubule formation (mitotic inhibitors), such as plant alkaloids (e.g., a compound belonging to a family of alkaline, nitrogen-containing molecules derived from plants that are biologically active and cytotoxic), e.g., taxanes, e.g., paclitaxel and docetaxel, and the vinka alkaloids, e.g., vinblastine, vincristine, and vinorelbine, and derivatives of podophyllotoxin; 3. Alkylating agents, such as nitrogen mustards, ethyleneimine compounds, alkyl sulphonates and other compounds with an alkylating action such as nitrosoureas, dacarbazine, cyclophosphamide, ifosfamide and melphalan; 4. Antimetabolites (nucleoside inhibitors), for example, folates, e.g., folic acid, fiuropyrimidines, purine or pyrimidine analogues such as 5-fluorouracil, capecitabine, gemcitabine, methotrexate and edatrexate; 5. Topoisomerase I inhibitors, such as topotecan, irinotecan, and 9-nitrocamptothecin, and camptothecin derivatives; and 6. Platinum compounds/complexes, such as cisplatin, oxaliplatin, and carboplatin; Exemplary chemotherapeutic agents for use in the methods of the invention include, but are not limited to, amifostine (ethyol), cisplatin, dacarbazine (DTIC), dactinomycin, mechlorethamine (nitrogen mustard), streptozocin, cyclophosphamide, carmustine (BCNU), lomustine (CCNU), doxorubicin (adriamycin), doxorubicin lipo (doxil), gemcitabine (gemzar), daunorubicin, daunorubicin lipo (daunoxome), procarbazine, mitomycin, cytarabine, etoposide, methotrexate, 5-fluorouracil (5-FU), vinblastine, vincristine, bleomycin, paclitaxel (taxol), docetaxel (taxotere), aldesleukin, asparaginase, busulfan, carboplatin, cladribine, camptothecin, CPT-11, 1O-hydroxy-7-ethyl-camptothecin (SN38), dacarbazine, S-I capecitabine, ftorafur, 5′deoxyfluorouridine, UFT, eniluracil, deoxycytidine, 5-azacytosine, 5-azadeoxycytosine, allopurinol, 2-chloro adenosine, trimetrexate, aminopterin, methylene-10-deazaminopterin (MDAM), oxaplatin, picoplatin, tetraplatin, satraplatin, platinum-DACH, ormaplatin, CI-973, JM-216, and analogs thereof, epirubicin, etoposide phosphate, 9-aminocamptothecin, 10,11-methylenedioxycamptothecin, karenitecin, 9-nitrocamptothecin, TAS 103, vindesine, L-phenylalanine mustard, ifosphamidemefosphamide, perfosfamide, trophosphamide carmustine, semustine, epothilones A-E, tomudex, 6-mercaptopurine, 6-thioguanine, amsacrine, etoposide phosphate, karenitecin, acyclovir, valacyclovir, ganciclovir, amantadine, rimantadine, lamivudine, zidovudine, bevacizumab, trastuzumab, rituximab, 5-Fluorouracil, Capecitabine, Pentostatin, Trimetrexate, Cladribine, floxuridine, fludarabine, hydroxyurea, ifosfamide, idarubicin, mesna, irinotecan, mitoxantrone, topotecan, leuprolide, megestrol, melphalan, mercaptopurine, plicamycin, mitotane, pegaspargase, pentostatin, pipobroman, plicamycin, streptozocin, tamoxifen, teniposide, testolactone, thioguanine, thiotepa, uracil mustard, vinorelbine, chlorambucil, cisplatin, doxorubicin, paclitaxel (taxol) and bleomycin, and combinations thereof which are readily apparent to one of skill in the art based on the appropriate standard of care for a particular tumor or cancer.

In another embodiment, an additional agent for use in the combination therapies of the invention is a biologic agent.

Biological agents (also called biologies) are the products of a biological system, e.g., an organism, cell, or recombinant system. Examples of such biologic agents include nucleic acid molecules (e.g., antisense nucleic acid molecules), interferons, interleukins, colony-stimulating factors, antibodies, e.g., monoclonal antibodies, anti-angiogenesis agents, and cytokines. Exemplary biologic agents are discussed in more detail below and generally belong to various classes including, for example: 1. Hormones, hormonal analogues, and hormonal complexes, e.g., estrogens and estrogen analogs, progesterone, progesterone analogs and progestins, androgens, adrenocorticosteroids, antiestrogens, antiandrogens, antitestosterones, adrenal steroid inhibitors, and anti-leuteinizing hormones; and 2. Enzymes, proteins, peptides, polyclonal and/or monoclonal antibodies, such as interleukins, interferons, colony stimulating factor, etc.

In one embodiment, the biologic is an interfereon. Interferons (IFN) are a type biologic agent that naturally occurs in the body. Interferons are also produced in the laboratory and given to cancer patients in biological therapy. They have been shown to improve the way a cancer patient's immune system acts against cancer cells.

Interferons may work directly on cancer cells to slow their growth, or they may cause cancer cells to change into cells with more normal behavior. Some interferons may also stimulate natural killer cells (NK) cells, T cells, and macrophages which are types of white blood cells in the bloodstream that help to fight cancer cells.

In one embodiment, the biologic is an interleukin. Interleukins (IL) stimulate the growth and activity of many immune cells. They are proteins (cytokines and chemokines) that occur naturally in the body, but can also be made in the laboratory.

Some interleukins stimulate the growth and activity of immune cells, such as lymphocytes, which work to destroy cancer cells.

In another embodiment, the biologic is a colony-stimulating factor.

Colony-stimulating factors (CSFs) are proteins given to patients to encourage stem cells within the bone marrow to produce more blood cells. The body constantly needs new white blood cells, red blood cells, and platelets, especially when cancer is present. CSFs are given, along with chemotherapy, to help boost the immune system. When cancer patients receive chemotherapy, the bone marrow's ability to produce new blood cells is suppressed, making patients more prone to developing infections. Parts of the immune system cannot function without blood cells, thus colony-stimulating factors encourage the bone marrow stem cells to produce white blood cells, platelets, and red blood cells.

With proper cell production, other cancer treatments can continue enabling patients to safely receive higher doses of chemotherapy.

In another embodiment, the biologic is an antibody. Antibodies, e.g., monoclonal antibodies, are agents, produced in the laboratory, that bind to cancer cells.

When cancer-destroying agents are introduced into the body, they seek out the antibodies and kill the cancer cells. Monoclonal antibody agents do not destroy healthy cells. Monoclonal antibodies achieve their therapeutic effect through various mechanisms. They can have direct effects in producing apoptosis or programmed cell death. They can block growth factor receptors, effectively arresting proliferation of tumor cells. In cells that express monoclonal antibodies, they can bring about anti idiotype antibody formation.

Examples of antibodies which may be used in the combination treatment of the invention include anti-insulin-like growth factor receptor-1, anti-CD20 antibodies, such as, but not limited to, cetuximab, Tositumomab, rituximab, and Ibritumomab. Anti-HER2 antibodies may also be used in combination with an environmental influencer for the treatment of cancer. In one embodiment, the anti-HER2 antibody is Trastuzumab (Herceptin). Other examples of antibodies which may be used in combination with an environmental influencer for the treatment of cancer include anti-CD52 antibodies (e.g., Alemtuzumab), anti-CD-22 antibodies (e.g., Epratuzumab), and anti-CD33 antibodies (e.g., Gemtuzumab ozogamicin). Anti-VEGF antibodies may also be used in combination with an environmental influencer for the treatment of cancer. In one embodiment, the anti-VEGF antibody is bevacizumab. In other embodiments, the biologic agent is an antibody which is an anti-EGFR antibody e.g., cetuximab. Another example is the anti-glycoprotein 17-1A antibody edrecolomab.

In another embodiment, the biologic is a cytokine. Cytokine therapy uses proteins (cytokines) to help a subject's immune system recognize and destroy those cells that are cancerous. Cytokines are produced naturally in the body by the immune system, but can also be produced in the laboratory. This therapy is used with advanced melanoma and with adjuvant therapy (therapy given after or in addition to the primary cancer treatment). Cytokine therapy reaches all parts of the body to kill cancer cells and prevent tumors from growing.

In another embodiment, the biologic is a fusion protein. For example, recombinant human Apo2L/TRAIL (Genentech) may be used in a combination therapy. Apo2/TRAIL is the first dual pro-apoptotic receptor agonist designed to activate both pro-apoptotic receptors DR4 and DR5, which are involved in the regulation of apoptosis (programmed cell death).

In one embodiment, the biologic is an antisense nucleic acid molecule.

As used herein, an “antisense” nucleic acid comprises a nucleotide sequence which is complementary to a “sense” nucleic acid encoding a protein, e.g., complementary to the coding strand of a double-stranded cDNA molecule, complementary to an mRNA sequence or complementary to the coding strand of a gene. Accordingly, an antisense nucleic acid can hydrogen bond to a sense nucleic acid.

In one embodiment, a biologic agent is an siRNA molecule, e.g., of a molecule that enhances angiogenesis, e.g., bFGF, VEGF and EGFR. In one embodiment, a biologic agent that inhibits angiogenesis mediates RNAi. RNA interference (RNAi) is a post-transcriptional, targeted gene-silencing technique that uses double-stranded RNA (dsRNA) to degrade messenger RNA (mRNA) containing the same sequence as the dsRNA (Sharp, P. A. and Zamore, P. D. 287, 2431-2432 (2000); Zamore, P. D., et al. Cell 101, 25-33 (2000). Tuschl, T. et al. Genes Dev. 13, 3191-3197 (1999); Cottrell T R, and Doering T L. 2003. Trends Microbiol. 11:37-43; Bushman F. 2003. Mol Therapy. 7:9-10; McManus M T and Sharp P A. 2002. Nat Rev Genet. 3.737-47). The process occurs when an endogenous ribonuclease cleaves the longer dsRNA into shorter, e.g., 21- or 22-nucleotide-long RNAs, termed small interfering RNAs or siRNAs. The smaller RNA segments then mediate the degradation of the target mRNA. Kits for synthesis of RNAi are commercially available from, e.g. New England Biolabs or Ambion. In one embodiment one or more chemistries for use in antisense RNA can be employed in molecules that mediate RNAi.

The use of antisense nucleic acids to downregulate the expression of a particular protein in a cell is well known in the art (see e.g., Weintraub, H. et al., Antisense RNA as a molecular tool for genetic analysis, Reviews—Trends in Genetics, Vol. 1(1) 1986; Askari, F. K. and McDonnell, W. M. (1996) N. Eng. J. Med. 334:316-318; Bennett, M. R. and Schwartz, S. M. (1995) Circulation 92:1981-1993; Mercola, D. and Cohen, J. S. (1995) Cancer Gene Ther. 2:47-59; Rossi, J J. (1995) Br. Med. Bull. 51.217-225; Wagner, R. W. (1994) Nature 372:333-335). An antisense nucleic acid molecule comprises a nucleotide sequence that is complementary to the coding strand of another nucleic acid molecule (e.g., an mRNA sequence) and accordingly is capable of hydrogen bonding to the coding strand of the other nucleic acid molecule. Antisense sequences complementary to a sequence of an mRNA can be complementary to a sequence found in the coding region of the mRNA, the 5′ or 3′ untranslated region of the mRNA or a region bridging the coding region and an untranslated region (e.g., at the junction of the 5′ untranslated region and the coding region). Furthermore, an antisense nucleic acid can be complementary in sequence to a regulatory region of the gene encoding the mRNA, for instance a transcription initiation sequence or regulatory element. Preferably, an antisense nucleic acid is designed so as to be complementary to a region preceding or spanning the initiation codon on the coding strand or in the 3′ untranslated region of an mRNA.

Given the coding strand sequences of a molecule that enhances angiogenesis, antisense nucleic acids of the invention can be designed according to the rules of Watson and Crick base pairing. The antisense nucleic acid molecule can be complementary to the entire coding region of the mRNA, but more preferably is an oligonucleotide which is antisense to only a portion of the coding or noncoding region of the mRNA. For example, the antisense oligonucleotide can be complementary to the region surrounding the translation start site of the mRNA. An antisense oligonucleotide can be, for example, about 5, 10, 15, 20, 25, 30, 35, 40, 45 or 50 nucleotides in length.

An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyl uracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N2-carboxypropyl) uracil, (acp3)w, and 2,6-diaminopurine. To inhibit expression in cells, one or more antisense oligonucleotides can be used. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

In yet another embodiment, the antisense nucleic acid molecule of the invention is an a-anomeric nucleic acid molecule. An a-anomeric nucleic acid molecule forms specific double-stranded hybrids with complementary RNA in which, contrary to the usual a-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In another embodiment, an antisense nucleic acid of the invention is a compound that mediates RNAi. RNA interfering agents include, but are not limited to, nucleic acid molecules including RNA molecules which are homologous to the target gene or genomic sequence, “short interfering RNA” (siRNA), “short hairpin” or “small hairpin RNA” (shRNA), and small molecules which interfere with or inhibit expression of a target gene by RNA interference (RNAi). RNA interference is a post-transcriptional, targeted gene-silencing technique that uses double-stranded RNA (dsRNA) to degrade messenger RNA (mRNA) containing the same sequence as the dsRNA (Sharp, P. A. and Zamore, P. D. 287, 2431-2432 (2000); Zamore, P. D., et al. Cell 101, 25-33 (2000). Tuschl, T. et al. Genes Dev. 13, 3191-3197 (1999)). The process occurs when an endogenous ribonuclease cleaves the longer dsRNA into shorter, 21- or 22-nucleotide-long RNAs, termed small interfering RNAs or siRNAs. The smaller RNA segments then mediate the degradation of the target mRNA. Kits for synthesis of RNAi are commercially available from, e.g. New England Biolabs and Ambion. In one embodiment one or more of the chemistries described above for use in antisense RNA can be employed.

Nucleic acid molecules encoding molecules that, e.g., inhibit angiogenesis, may be introduced into the subject in a form suitable for expression of the encoded protein in the cells of the subject may also be used in the methods of the invention. Exemplary molecules that inhibit angiogenesis include, but are not limited to, TSP-I, TSP-2, IFN-g, IFN-a, angiostatin, endostatin, tumastatin, canstatin, VEGI, PEDF, vasohibin, and the 16 kDa fragment of prolactin 2-Methoxyestradiol (see, Kerbel (2004) J. Clin Invest 114:884, for review).

For example, a full length or partial cDNA sequence is cloned into a recombinant expression vector and the vector is transfected into a cell using standard molecular biology techniques. The cDNA can be obtained, for example, by amplification using the polymerase chain reaction (PCR) or by screening an appropriate cDNA library. The nucleotide sequences of the cDNA can be used for the design of PCR primers that allow for amplification of a cDNA by standard PCR methods or for the design of a hybridization probe that can be used to screen a cDNA library using standard hybridization methods. Following isolation or amplification of the cDNA, the DNA fragment is introduced into a suitable expression vector.

Exemplary biologic agents for use in the methods of the invention include, but are not limited to, gefitinib (Iressa), anastrazole, diethylstilbesterol, estradiol, premarin, raloxifene, progesterone, norethynodrel, esthisterone, dimesthisterone, megestrol acetate, medroxyprogesterone acetate, hydroxyprogesterone caproate, norethisterone, methyltestosterone, testosterone, dexamthasone, prednisone, Cortisol, solumedrol, tamoxifen, fulvestrant, toremifene, aminoglutethimide, testolactone, droloxifene, anastrozole, bicalutamide, flutamide, nilutamide, goserelin, flutamide, leuprolide, triptorelin, aminoglutethimide, mitotane, goserelin, cetuximab, erlotinib, imatinib, Tositumomab, Alemtuzumab, Trastuzumab, Gemtuzumab, Rituximab, Ibritumomab tiuxetan, Bevacizumab, Denileukin diftitox, Daclizumab, interferon alpha, interferon beta, anti-4-1BB, anti-4-1BBL, anti-CD40, anti-CD 154, anti-OX40, anti-OX40L, anti-CD28, anti-CD80, anti-CD86, anti-CD70, anti-CD27, anti-HVEM, anti-LIGHT, anti-GITR, anti-GITRL, anti-CTLA-4, soluble OX40L, soluble 4-IBBL, soluble CD154, soluble GITRL, soluble LIGHT, soluble CD70, soluble CD80, soluble CD86, soluble CTLA4-Ig, GVAX®, and combinations thereof which are readily apparent to one of skill in the art based on the appropriate standard of care for a particular tumor or cancer. The soluble forms of agents may be made as, for example fusion proteins, by operatively linking the agent with, for example, Ig-Fc region.

It should be noted that more than one additional agent, e.g., 1, 2, 3, 4, 5, may be administered in combination with a CoQ10 molecule. For example, in one embodiment two chemotherapeutic agents may be administered in combination with a CoQ10 molecule. In another embodiment, a chemotherapeutic agent, a biologic agent, and a CoQ10 molecule may be administered.

Various forms of the biologic agents may be used. These include, without limitation, such forms as proform molecules, uncharged molecules, molecular complexes, salts, ethers, esters, amides, and the like, which are biologically activated when implanted, injected or otherwise inserted into the tumor.

The present invention is further illustrated by the following examples which should not be construed as limiting in any way. The contents of all cited references, including literature references, issued patents, and published patent applications, as cited throughout this application are hereby expressly incorporated by reference.

### EXEMPLIFICATION OF THE INVENTION

### Example 1

### Identification of CoQ10 as a MIM

In order to evaluate CoQ10 as a potential MIM, CoQ10 in oxidized form was exogenously added to a panel of cell lines, including both cancer cell lines and normal control cell lines, and the changes induced to the cellular microenvironment profile for each cell line in the panel were assessed. Changes to cell morphology/physiology, and to cell composition, including both mRNA and protein levels, were evaluated and compared for the diseased cells as compared to normal cells. The results of these experiments identified CoQ10 and, in particular, the oxidized form of CoQ10, as a MIM.

In a first set of experiments, changes to cell morphology/physiology were evaluated by examining the sensitivity and apoptotic response of cells to CoQ10. A panel of skin cell lines including a control cell lines (primary culture of keratinocytes and melanocytes) and several skin cancers cell lines (SK-MEL-28, a non-metastatic skin melanoma; SK-MEL-2, a metastatic skin melanoma; or SCC, a squamous cell carcinoma; PaCa2, a pancreatic cancer cell line; or HEP-G2, a liver cancer cell line) were treated with various levels of Coenzyme Q10. The results of these experiments demonstrated that the cancer cell lines exhibited an altered dose dependent response as compared to the control cell lines, with an induction of apoptosis and cell death in the cancer cells only.

Assays were next employed to assess changes in the composition of the cell following treatment with CoQ10. Changes in gene expression at the mRNA level were analyzed using Real-Time PCR array methodology. In complementary experiments, changes in gene expression at the protein level were analyzed by using antibody microarray methodology, 2-dimensional gel electrophoresis followed by protein identificuation using mass spectrometry characterization, and by western blot analysis. The results from these assays demonstrated that significant changes in gene expression, both at the mRNA and protein levels, were induced in the cell lines examined due to the addition of the oxidized form of CoQ10. Genes modulated by CoQ10 treatment were found to be clustered into several cellular pathways, including apoptosis, cancer biology and cell growth, glycolysis and metabolism, molecular transport, and cellular signaling.

Experiments were carried out to confirm the entry of CoQ10 into cells and to determine the level and form of CoQ10 present in the cells. In particular, the level of Coenzyme Q10, as well as the form of CoQ10 (i.e., oxidized or reduced), present in the mitochondria was determined by analyzing mitochondrial enriched preparations from cells treated with CoQ10. The level of Coenzyme Q10 present in the mitochondria was confirmed to increase in a time and dose dependent manner with the addition of exogenous Q10. In a surprising and unexpected result, CoQ10 was determined to be present in the mitochondria primarily in oxidized form. In addition, changes in levels of proteins from mitochondria enriched samples were analyzed by using 2-D gel electrophoresis and protein identification by mass spectrometry characterization. The results from these experiments demonstrated that the levels of the oxidized form of CoQ10 in the mitochondria over the time course examined correlated with a wide variety of cellular changes, as evidenced by the modulation of mRNA and protein levels for specific proteins related to metabolic and apoptotic pathways.

The results described by Applicants herein identified the endogenous molecule CoQ10 and, in particular, the oxidized form of CoQ10, as a MIM. For example, the results identified CoQ10 as a MIM, since CoQ10 was observed to induce changes in gene expression at both the mRNA and protein level. The results identified CoQ10 as having multidimensional character, since CoQ10 induced differential changes in cell morphology/physiology and cell composition (e.g., differential changes in gene expression at both the mRNA and protein level), in a disease state (e.g., cancer) as compared to a normal (e.g., non-cancerous) state. Moreover, the results identified CoQ10 as having multidimensional character in that CoQ10 was capable of entering a cell, and thus exhibited both therapeutic and carrier effects.

### Example 2

### Methods for Identifying Relevant Processes and Biomarkers for Sarcomas

From the cell based assays in which cell lines, e.g., sarcoma cell lines, were treated with a molecule of interest, the differences in treated vs non-treated cells is evaluated by mRNA arrays, protein antibody arrays, and 2D gel electrophoresis. The proteins identified from comparative sample analysis to be modulated by the MIM or Epi-shifter, e.g., CoQ10, are evaluated from a Systems Biology perspective with pathway analysis (Ingenuity IPA software) and a review of the known literature. Proteins identified as potential therapeutic or biomarker targets are submitted to confirmatory assays such as Western blot analysis, siRNA knock-down, or recombinant protein production and characterization methods.

### Example 3

### Relative Sensitivities of Oncogenic and Normal Cells to Coenzyme Q10

The effects of Coenzyme Q10 treatment on a variety of oncogenic and normal cell lines were examined and compared. The sensitivity of cells to Coenzyme Q10 was assessed by monitoring induction of apoptosis. CoQ10 treatment of cells was carried out as described in detail below in the Materials and Methods. Induction of apoptosis was assessed in the treated cells by monitoring indicators of early apoptosis (e.g., Bcl-2 expression, caspase activation and by using annexin V assays) as described below. From these studies, the minimal CoQ10 dosage, e.g., concentration of CoQ10 and time of treatment, required to induce apoptosis in the panel of cell lines was determined.

In an unexpected and surprising result, the data demonstrated that efficacy of Coenzyme Q10 treatment was greater in cell types that exhibited increased oncogenicity and/or greater metastatic potential, i.e., cell types that were derived from more aggressive cancers or tumors. The results of these studies are summarized below in Table 1. The data demonstrates that CoQ10 is more effective in both a time and concentration dependent manner on cells in a more aggressive cancer state. Moreover, a surprising divergent effect was observed on normal cells as compared to oncogenic cells. Specifically, Coenzyme Q10 was unexpectedly found to exhibit a slightly supportive role in a normal tissue environment, wherein increased proliferation and migration was observed in normal cells, including keratinocytes and dermal fibroblasts.

The effect of Coenzyme Q10 on gene regulatory and protein mechanisms in cancer is different in a normal cell. Key cellular machinery and components, such as cytoskeletal architecture, membrane fluidity, transport mechanisms, immunomodulation, angiogenesis, cell cycle control, genomic stability, oxidative control, glycolytic flux, metabolic control and integrity of extracellular matrix proteins, are dysregulated and thus the genetic and molecular fingerprint of the cell is altered. The disease environment favors governance of cellular control processes. The data provided herein suggests that CoQ10 exerts a greater level of efficacy (e.g., in cancer cells vs. normal cells, and in cells of a more aggressive cancer state as compared to cells 1 of a less aggressive or non-aggressive cancer state) by normalizing some of the key aforementioned processes in a manner that allows for restored apoptotic potential.

TABLE 1
Minimal CoQ10 concentration and treatment time required for induction of
early apoptosis in various cell types.

| | Indication | | | Level of |
|---|---|---|---|---|
| | of Early | | | aggressiveness: |
| | apoptosis | | | 1 = normal |
| | (Bcl-2, annexin | Concen- | | tissue |
| Tissue Origin | V, or caspase | tration | Time | 2 = malignant |
| (Cell type) | activation) | (μM) | (hr) | 3 = metastatic |
| SKIN: | | | | |
| Keratinocytes (Heka, | None | N/A | N/A | 1 |
| Hekn) | | | | |
| Fibroblasts (nFib) | None | N/A | N/A | 1 |
| Melanocytes (Hema, | None | N/A | N/A | 1 |
| LP) | | | | |
| Melanoma | Strong | 20 | 24 | 2 |
| (Skmel 28) | | | | |
| Melanoma (Skmel 2) | Very Strong | 25 | 24 | 3 |
| SCC, Squamous cell | Very Strong | 25 | 24 | 3 |
| carcinoma | | | | |
| BREAST: | | | | |
| MCF-7 | Strong | 50 | 48 | 2 |
| SkBr-3 | Very Strong | 50 | 24 | 3 |
| BT-20 | Strong | 100 | 48 | 2 |
| ZR-75 | Slight | 200 | 72 | 2 |
| MDA MB 468 | Strong | 100 | 48 | 2 |
| Mammary fiboblasts: | None | N/A | | 1 |
| 184A1 and 184B5) | | | | |
| (Lawrence Berkeley) | | | | |
| PROSTATE: | | | | |
| PC3 | Very Strong | 25 | 24 | 3 |
| LIVER: | | | | |
| HepG2 | Very Strong | 50 | 24 | 3 |
| Hep3B | Very Strong | 50 | 24 | 3 |
| BONE: | | | | |
| Osteosarcoma (143b) | Very Strong | 50 | 48 | 2 |
| Ewing's sarcoma | Extremely | 5 | 1 | 3 |
| (NCI) | strong | | | |
| PANCREAS: | | | | 3 |
| PaCa2 | Very Strong | 25 | 24 | |
| Heart: | | | | |
| Aortic smooth muscle | None | N/A | N/A | 1 |
| (HASMC) | | | | |

### Materials and Methods

### Cell Preparation and Treatment

### Cells Prepared in Dishes or Flasks

Cells were cultured in T-75 flasks with relevant medium supplemented with 10% Fetal Bovine Serum (FBS), 1% PSA (penicillin, streptomycin, amphotericin B) (Invitrogen and Cellgro) in a 37° C. incubator with 5% CO₂ levels until 70-80% confluence was reached. To harvest cells for treatment, flasks were primed with 1 mL Trypsin, aspirated, trypsinized with an additional 3 mL, and incubated at 37° C. for 3-5 minutes. Cells were then neutralized with an equal volume of media and the subsequent solution was centrifuged at 10,000 rpm for 8 minutes. The supernatant was aspirated and the cells were resuspended with 8.5 ml of media. A mixture of 500 ul of the resuspension and 9.5 ml of isopropanol was read twice by a coulter counter and the appropriate number of cells to be seeded into each dish was determined. Control and concentration ranging from 0-200 μM groups were examined in triplicate. From a 500 μM CoQ-10 stock solution, serial dilutions were performed to achieve desired experimental concentration in appropriate dishes. Dishes were incubated in a 37° C. incubator with 5% CO₂ levels for 0-72 hours depending on cell type and experimental protocol.

### Protein Isolation and Quantification

### Cells Prepared in Dishes

Following cell treatment incubation period was complete, protein isolation was performed. Dishes of all treatment groups were washed twice with 2 ml, and once with 1 ml of ice cold 1× Phosphate Buffered Saline (PBS). The PBS was aspirated from the dishes after the initial 2 washes only. Cells were gently scraped and collected into microcentrifuge tubes using the final volume from the third wash and centrifuged at 10,000 rpm for 10 minutes. After centrifugation, the supernatant was aspirated and the pellet was lysed with 50 uL of lysis buffer (1 uL of protease and phosphotase inhibitor for every 100 uL of lysis buffer). Samples were then frozen overnight at −20° C.

### Cells Prepared in Flasks

After the cell treatment incubation period was complete, protein isolation was performed. Flasks of all treatment groups were washed twice with 5 mL, and once with 3 mL of ice cold 1×PBS. The PBS was aspirated from the flasks after the first 2 washes only. Cells were gently scraped and collected into 15 mL centrifuge tubes using the final volume from the third wash and centrifuged for at 10,000 rpm for 10 minutes. After centrifugation, the supernatant was aspirated and the pellet was lysed with an appropriate amount of lysis buffer (1 uL of protease and phosphotase inhibitor for every 100 uL of lysis buffer). Lysis buffer volume was dependent on pellet size. Samples were transferred in microcentrifuge tubes and frozen overnight at −20° C.

### Protein Quantification

Samples were thawed at −4° C. and sonicated to ensure homogenization the day following protein isolation. Protein quantification was performed using the micro BCA protein assay kit (Pierce). To prepare samples for Immuno-blotting, a 1:19 solution of betamercaptoethanol (Sigma) to sample buffer (Bio-Rad) was prepared. Samples were diluted 1:1 with the betamercaptoethanol-sample buffer solution, boiled at 95° C. for 5 minutes, and frozen overnight at −20° C.

### Immuno-Blotting

### Bcl-2, Caspase, 9, Cyotochrome c

The volume of sample to load per well was determined using the raw mean concentration of protein obtained from the BCA protein assay. Approximately 30-60 μg of protein were loaded for each treatment time point. Proteins were run in triplicate on 12% Tris-HCl ready gels (Bio-Rad) or hand cast gels in 1× running buffer at 85 and 100 volts. Proteins were then transferred onto nitrocellulose paper for an hour at 100 volts, and blocked for another hour in a 5% milk solution. Membranes were placed in primary antibody (1 uL Ab:1000 uL TBST) (Cell Signaling) overnight at −4° C. The following day, membranes were washed three times for ten minutes each with Tris-Buffered Saline Tween-20 (TBST), and secondary antibody (anti-rabbit; 1 uL Ab: 1000 uL TBST) was applied for an hour at −4° C. Membranes were washed again three times for ten minutes with TBST and chemoluminescence using Pico or Femto substrate was completed (Pierce). Membranes were then developed at time intervals that produced the best visual results. After developing, membranes were kept in TBST at −4° C. until Actin levels could be measured.

### Actin

Membranes were placed in primary Actin antibody (1 uL Ab:5000 uL TBST) (cell signaling) for 1 hour at −4° C., washed three times for ten minutes each with TBST, and secondary antibody (anti-mouse; 1 uL Ab: 1000 uL TBST) was applied for an hour at −4° C. Membranes were washed again three times for ten minutes each with TBST and chemoluminescence using Pico substrate was completed (Pierce). Membranes were then developed at time intervals that produced the best visual results.

Annexin V assay

Cells were washed twice in PBS10× and resuspended in Binding Buffer (0.1 M HEPES, pH 7.4; 1.4 M NaCl; 25 mM CaCl2). Samples of 100 μl were added to a culture tube with 5 μl of annexin-PE dye or 7-ADD. The cells were mixed and incubated without light at room temperature for 15 minutes. After which, 400 μl of 1×Binding Buffer was added to each sample and they were subjected to analysis by flow cytometry.

In Examples 4-7, below, the goal was to gain insights into mechanisms of CoQ10 action particular to the NCIES0808 cells. The NCIES0808 cell line is directly derived from a patient with Ewing's sarcoma and hence is the most relevant cell line to be used in the study. The thought underlying the project is that this study will be beneficial to the development of the API and present to the community a better understanding of its actions.

The intent of the experiments is to characterize changes within the cellular environment at the RNA and the protein level based on the following experiments. (1) PCR arrays

Angiogenesis

Diabetes

Mitochondrial

### (2) Antibody Arrays

(3) 2D gel analysis

### (4) Western Analysis

### Materials and Methods for Examples 4-8

### Coenzyme Q10 Stock

A 500 μM Coenzyme Q10 (5% isopropanol in cell growth media) was prepared as follows. A 10 mL 500 μM Coenzyme Q10 stock was made fresh every time.

### Molecular Weight: 863.34

(0.0005 mol/L)(0.010 L)(863.34 g/mol)=0.004317 g

To make 10 mL of 500 μM stock, 4.32 mg Coenzyme Q10 was weighted out in a 15 mL falcon tube, and 500 μL isopropanol was added. The solution was warmed in a 50-60° C. water bath while swirling to dissolve completely. To this solution, 9.5 mL of media (the same media in which the cells are grown) was added.

### NCIES0808 Cells.

NCIES0808 cells were grown in DMEM/F12 containing glutamax and 17 mM glucose along with 5% FBS, Penstrep and Amphotericin. Cells were passaged to obtained sufficient volume for the experiments.

### Coenzyme Q10 Treatment and Total Protein Isolation

Supplemented media was conditioned with Q10 to 50 and 100 micro molar concentrations. Cells were treated with control, 50 μM Q10, and 100 μM Q10 in triplicate. Protein was isolated from the treated and control flask after 3, 6 or 24 hours. For isolation of proteins, cells were washed three times with 5 mL of ice cold PBS at a pH of 7.4. The cells were then scraped in 3 mL of PBS, pelleted by centrifuge, and re-suspended in a lysis buffer at pH 7.4 (80 mM TRIS-HCl, 1% SDS, with protease and phosphotase inhibitors). Protein concentrations were quantified using the BCA method.

### RNA Isolation:

Cells were lysed for RNA isolation at different treatment times using the RNeasy Mini kit (Qiagen, Inc., Valencia Calif.) kit following the manufacturer's instructions. RNA was quantified by measuring Optical Density at 260 nm.

### First Strand Synthesis:

First strand cDNA was synthesized from 1 μg of total RNA using the RT2 First Strand Synthesis kit (SABiosciences., Frederick Md.) as per manufacturer's recommendations.

### Real-Time PCR:

Products from the first strand synthesis were diluted with water, mixed with the SYBR green master mix (SABiosciences., Frederick Md.) and loaded onto PCR arrays. Real time PCR was run on the PCR Arrays (Apoptosis Arrays, Diabetes Arrays, Oxidative stress and Antioxidant defense Arrays and Heat Shock Protein Arrays.) (SABiosciences, Frederick Md.) on a Biorad CFX96.

### PCR Arrays:

NCIES0808 cells were plated in T25 flasks at a density of 2×10⁶ cells per flask in media or media containing 50 uM/100 uM Q10. All treatment groups were run in triplicate. Cells were harvested at 0, 3, 6, 24 or 48 hours. Pictures were taken to examine cell morphology before harvesting. To harvest cells, media was removed but saved to be able to collect floating apoptotic cells. Cells were trypsinized with 1 ml of trypsin-EDTA and the enzyme action was stopped by addition of 4 ml complete media. Trypsinized cells were added to the appropriate tube containing the media with dead cells. Cells were centrifuged at 1200 rpm for 5 minutes and media was aspirated leaving behind the cell pellet for RNA extraction. RNA isolation from cell pellets was carried out with the RNeasy kit (Qiagen, Valencia Calif.) according to the manufacturer's instructions. RNA samples were eluted from spin columns in water; absorbance was measured at 260 nm, 230 nm and 280 nm. The purity of RNA was evaluated by the 260/230 and 280/230 ratios. The concentration of RNA in all of the samples was calculated from absorbance values at 230 nm. First strand cDNA was synthesized from 0.5 ug of all RNA samples using instructions provided with the First strand kit (SABiosciences, Frederick, Md.). The synthesized first strand from a sample was dispensed equally in a PCR array plate containing primers within a pathway (Angiogenesis, Diabetes and Mitochondria) (SABiosciences Corporation, Frederick, Md.). The arrays were amplified with real time PCR using the SYBR green detection methods using manufacturer approved protocols. The ct values from each of the samples were normalized to three housekeeping genes and fold regulation of Q10 treated groups was compared to time matched controls from cells grown in regular media was calculated.

### Sample Preparation for Proteomics:

NCIES0808 cells were plated in T25 flasks in experimental conditions similar to those described in the PCR array section. At the end of the treatment time, cells were trypsinized as described in the PCR array section and washed twice in ice cold TBS and snap frozen in liquid nitrogen. Further processing for Western blots was carried out at UMass.

NCIES0808 cells were treated with Q10 separately in larger volumes for isolation of sufficient mitochondria for proteomic analysis. Cells were treated with media, 50 uM Q10 or 100 uM Q10 for 0, 3, 6, 24 and 48 hours in T175 flasks. Two flasks were grown for each condition and cells from the two were pooled during harvesting. After the required treatment time, cells were trypsinized and washed twice in ice cold TBS. Pelleted cells were snap frozen in liquid nitrogen and frozen at −80° C. until mitochondria were isolated. Mitochondria were isolated using manufacturers instructions available with the MitoProfile Mitosciences Isolation Kit for Cultured Cells (Mitosciences Inc, Eugene, Oreg.).

### Western Blots Preparation:

Cells were grown and treated with CoQ10 at 50 uM and 100 uM, along with the proper controls. The total cell lysates (as prepared above) were processed and evaluated by Western blot analysis. Proteins from each treatment group were resolved on SDS-PAGE and transferred onto PVDF membranes. They were then hybridized with antibodies.

### Immunoblotting:

Either 5 or 10 μg of protein was assayed per sample by immunoblotting. Proteins were separated on 10-20% Tris-HCl gels or 4-12% Bis-Tris gels, transferred via electrophoresis to PVDF membranes and blocked using a 5% GE/Amersham ECF blocker and TBST solution prior to incubation with primary antibodies. The primary antibodies were incubated overnight at 4 degrees C. in a 5% BSA and TBST solution. Secondary antibodies were incubated for one hour at room temperature. All antibodies were purchased from commercial vendors. Antibodies were used at the manufacturers' recommended dilution, with the control βActin at a dilution of 1:5000. Blots were developed using GE/Amersham ECF reagent, and results were quantified using the Fuji FL-5100 laser scanner and Bio-Rad Quantity One densitometry analysis software. All blots were also probed for and normalized to their respective βActin expression.

### Two-Dimensional Electrophoresis:

Before isoelectric focusing (IEF), samples were solubilized in 40 mM Tris, 7 M urea, 2 M thiourea, and 1% C7 zwitterionic detergent, reduced with tributylphosphine, and alkylated with 10 mM acrylamide for 90 min at room temperature. After the sample was run through a 10-kDa cutoff Amicon Ultra device with at least 3 volumes of the resuspension buffer, consisting of 7 M urea, 2 M thiourea, and 2% CHAPS to reduce the conductivity of the sample. One hundred micrograms of protein were subjected to IEF on 11-cm pH 3 to 10, pH 4 to 7 or pH 6 to 11 immobilized pH gradient strips (GE, Amersham, USA) to 100,000 volts hour. After IEF, immobilized pH gradient strips were equilibrated in 6 M urea, 2% SDS, 50 mM Tris-acetate buffer, pH 7.0, and 0.01% bromphenol blue and subjected to SDS-polyacrylamide gel electrophoresis on 8 to 16% Tris-HCl Precast Gel, 1 mm (Bio-Rad, USA). The gels were run in duplicate. They were fixed, stained in SYPRO Ruby, 80 mL/gel (Invitrogen, USA) and imaged on Fuji FLA-5100 laser scanner.

### Image Analysis:

Analysis of all gel images was performed using Progenesis Discovery and Pro (Nonlinear Dynamics Inc., Newcastle upon Tyne, UK). After spot detection, matching, background subtraction, normalization, and filtering, data for SYPRO Ruby gel images was exported. Pairwise comparisons between groups were performed using the Student's t test in Progenesis Discovery to identify spots whose expression was significantly altered (p>0.05). Manual annotation of each statistically significant spots was performed to assure accurate detection.

### Mass Spectrometry:

Tryptic peptides extracted from respective gel plugs were dried down to a 10 ul volume and acidified with 1-2 ul of 1% TFA. Samples were loaded on an uC18 Zip Tip (Millipore, Corp) after pre-equilibration in 0.1% TFA. After washing with 2×10 ul aliquots of 0.1% TFA, samples were deposited directly onto the MALDI sample target using 1 ul of Matrix solution 15 mg/ml of 2,5 Dihydroxybenzoic Acid (MassPrep DHB, Waters Corp.) in 50:50 Acetonitrile: 0.1% TFA. Samples were allowed to air dry prior to insertion into the mass spectrometer. Analysis were performed on a Kratos Axima QIT (Shimadzu Instruments) matrix-assisted-laser desorption/ionization (MALDI) mass spectrometer. Peptides were analyzed in positive ion mode in mid mass range (700-3000 Da). The instrument was externally calibrated with Angiotensin II (1046.54), P14R (1533.86) and ACTH (18-39) 2465.20 Da. Precursors were selected based on signal intensity at a mass resolution width of 250 for CID fragmentation using Argon as the collision gas. Database searches were performed in house with Mascot (Matrix Sciences, Ltd.) using the Peptide Mass Fingerprint program for MS data and the MS/MS Ion Search program for CID data. All identifications were confirmed or established with CID (MS/MS) data.

### Antibody Arrays:

NCIES0808 cells were received from SBH in T165 flasks (×55). The cells were approximately 90-95% confluent and the media had a typical pink color. The cell morphology was examined closely under a microscope and the cells were noted to appear healthy with no visual signs of contamination or intracellular inclusions.

A 500 μM Q10 stock was made using the same protocol outlined for the PCR arrays. The media was exchanged in every flask with 50 μM and 100 μM Q10 media being placed into the appropriate flasks. The cells were incubated for 3 hr and 6 hr in the Q10 formulated media and the cells were harvested. Each flask was washed with 10 ml of ice cold PBS and trypsinized with 5 ml of trypsin-EDTA. The cells were harvested by gentle pipetting and the enzyme action was stopped by addition of 30 ml complete media. The cells were centrifuged at 1200 rpm for 5 minutes and media was aspirated from the tube leaving behind the cell pellet for protein extraction.

The proteins were extracted from the cells as per page 2; sub-category IA; of the manufacture's Product Information Sheet, Sigma®, Panarama® Antibody Microarray EPRESS Profiler725, cat#: XP725. The protein material from the whole cell lysates was conjugated with Cy3 and Cy5 dyes, GE Healthcare, product #: 25-8009-86 Cy3 and 25-8009-87 Cy5 as per the manufacturer's instructions outlined in the above mentioned product sheet sub-category IIA. The Antibody Array chips prepared once again following the manufacturer's instructions given in sub-category III of the product sheet and left to dry for 24 hr. in a dark room. The arrays were analyzed using a Fuji FLA-5100 UV scanner at the 532 nm for the Cy3 dye and 635 nm for the Cy5 dye. Data was collected on media only, 50 μM Q10 and 100 μM Q10 samples at 3 hr. and 6 hr. all in triplicate.

### IPA Analysis:

The output from the experiments described below was combined together using Ingenuity Pathway Analysis (http://www.ingenuity.com) as a tool to elucidate potential pathways modulated by Q10.

### Example 4

### Sensitivity of NCI-ES-0808 Cells to CoQ10 Treatment

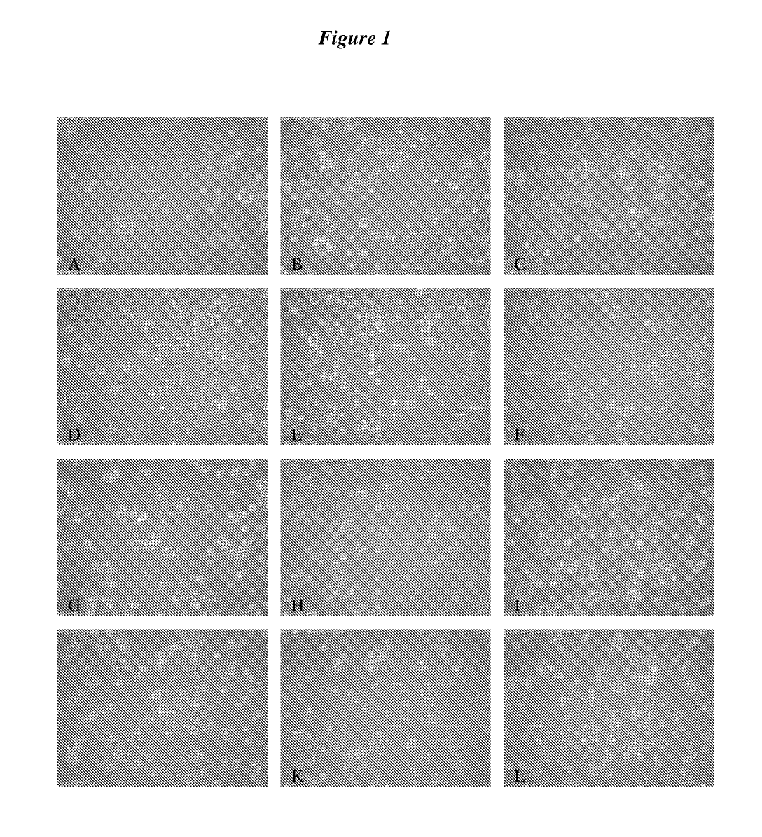

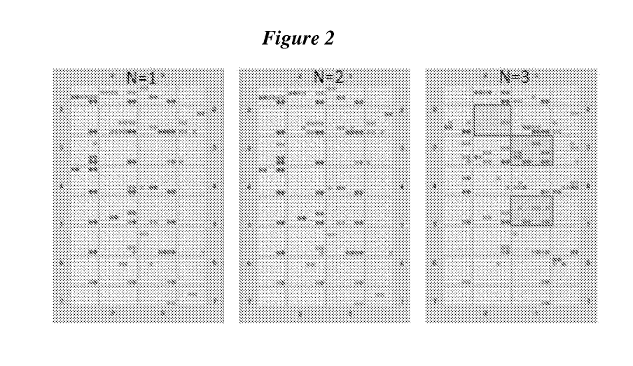

The morphology of NCI-ES-0808 cells was monitored following treatment with CoQ10. Pictures of NCI-ES-0808 cells were taken through the microscope 3, 6, 24 or 48 hours after Q10 treatment and just prior to harvesting. Cells were partially attached at 3 hours after treatment, but by six hours, they appeared to be completed attached. No differences in morphology, number of visually ascertainable apoptotic cells or cell number seemed apparent by microscopy among treatment groups during the time scale of the experiment which was 3 hrs and 6 hrs post treatment (FIG. 1).

### Example 5

### Real-Time PCR Arrays

The experiments described in this example were performed to test the overall hypothesis that Q10 would have an impact on expression of multiple genes in Ewing's sarcoma cells. The mRNA from NCIES0808 cells treated with 50 μM or 100 μM Q10 for various times was evaluated by RT-PCR against a panel of target proteins involved in human diabetes, human angiogenesis or human mitochondrial pathways.

Ct values obtained from a real time thermocycler were loaded onto the analysis tool on the SABiosciences website for calculation of fold regulation compared to cells with media. The genes that are modulated by CoQ10 on analysis of the Human Diabetes Arrays are summarized in Table 2. The genes that are modulated by CoQ10 on analysis of the Human Angiogenesis Arrays are summarized in Table 3. The genes that are included in the tables below are those that show a p value of close to 0.05. Analysis of the Human Mitochondrial arrays did not reveal any modulated genes at the CoQ10 doses and time points examined.

TABLE 2
Genes from Human Diabetes Arrays
Regulated in Major mRNA level changes to NCIES0808 cells
treated with 100 μM CoQ10.

| Gene | Pattern of regulation. |
|---|---|
| ADRB | Upregulation at 24 hours with 100 uM Q10. |
| CEACAM1 | Upregulation at 48 hours with 100 uM Q10. |
| DUSP4 | Upregulation at 24 hours with 100 uM Q10. |
| FOX C2 | Upregulation at 24 hours with 100 uM Q10 |
| FOXP3 | Upregulation at 6 hours with 50 uM and 100 uM Q10. |
| GCGR | Upregulation at 6 hours with 100 uM Q10. |
| GPD1 | Upregulation at 6 hours with 100 uM Q10. |
| HMOX1 | Upregulation at 24 and 48 hours with 100 uM Q10. |
| IL4R | Upregulation at 48 hours with 100 uM Q10. |
| INPPL1 | Upregulation at 6 hours with 100 uM Q10. |
| IRS2 | Upregulation at 6 hours with 100 uM Q10. |
| VEGFA | Upregulation at 24 hours with 100 uM Q10 and 48 hours |
| | with 50 uM Q10. |

TABLE 3
Genes from Human Angiogenesis Arrays
Regulated in Major mRNA level changes to NCIES0808 cells
treated with 100 μM CoQ10.

| Gene | Pattern of regulation. |
|---|---|
| ANGPTL3 | Down regulation at 3 hours with 100 uM Q10. |
| CCL2 | Down regulation at 3 hours with 100 uM Q10. |
| CDH5 | Down regulation at 3 and 24 hours with 100 uM Q10. |
| CXCL1 | Down regulation at 3 hours with 100 uM Q10 |
| CXCL3 | Down regulation at 3 hours with 100 uM Q10. |
| LAMA5 | Up regulation at 6 hours with 100 uM Q10. |
| PXLDC1 | Up regulation at 48 hours with 100 uM Q10. |

### Example 6

### Antibody MicroArray Analysis

The evaluation of changes in protein concentration due to the presence of Q10 was evaluated through the utilization of antibody microarray methods. The microarray contained antibodies for over 700 proteins, sampling a broad range of protein types and potential pathway markers.

For an initial analysis of the efficiency and reproducibility of chip preparation a general overview of each chip (n=1, 2, 3) for all data sets was performed. A pattern analysis of the 50 μM Q10, 3 hr data series shows that although n=1 and n=2 are very similar n=3 has a much different pattern. For this reason the n=3 data was disregarded in the statistical evaluation of the array data.

Once data sets were collected for all arrays, the data was scrutinized against three major parameters. First the data was normalized using the summed fluorescent intensities method described in sub-category V of the manufactures instructions. After the normalization process any data points with a zero value for the normalized Cy3/Cy5 ratio were considered statistically irrelevant and removed from the test set. An evaluation of the positive and negative Cy3/Cy5 data (included as controls on the chip) and a visual inspection of the spectral density for a given spot it was determined that and array data point with a spectral density less than 10 was approaching the background level and deemed statistically irrelevant and removed from the data series. The resulting data was considered the base data set for further evaluation. Each data set was sorted according to the normalized spectral density ratio and the top 45 up-regulated and down-regulated proteins were evaluated. Only the proteins that noted to appear in the all replicate studies (n=1, 2, 3) were nominated as being statistically relevant and fall within the 95% confidence range of these statistical evaluations. It should be noted that there was a significant variance within each data set of the 3 hr. time trials. It is likely that at this time point the cells have not converged to a point where conclusions can be drawn from the data with a high percent of statistical relevance. However the data obtained from the 6 hr. time points satisfy all of our statistical analysis and are present in a replicate experiments (n=1, 2, 3) and the data for these analysis are presented below in Tables 4 and 5.

TABLE 4
Proteins Upregulated in NCIES0808 cells
treated for 6 Hours with 50 or 100 μM CoQ10.

| | 50 μM CoQ10 6 hr | 100 μM CoQ10 6 hr |
|---|---|---|
| 1 | p300 CBP | FKHR FOXO1a |
| 2 | P53R2 | MDM2 |
| 3 | Phosphatidylserine Receptor | Fas Ligand |
| 4 | Cytokeratin Peptide 17 | P53R2 |
| 5 | Cytokeratin peptide 13 | Caspase 10 |
| 6 | Neurofilament 160 200 | Crk2 |
| 7 | Rab5 | Cdc 6 |
| 8 | Filensin | P21 WAF 1 Cip 1 |
| 9 | P53R2 | ASPP 1 |
| 10 | MDM2 | HDAC 4 |
| 11 | MSH6 | Cyclin B1 |
| 12 | Heat Shock Factor 2 | CD 40 |
| 13 | AFX | GAD 65 |
| 14 | FLIPg d | TAP |
| 15 | JAB 1 | Par4 (prostate apoptosis response 4) |
| 16 | Myosine | MRP1 |
| 17 | MEKK4 | |
| 18 | cRaf pSer621 | |
| 19 | PDK 1 | |
| 20 | Caspase 12 | |
| 21 | Phospholipase D1 | |
| 22 | P34 cdc2 | |
| 23 | P53 BP1 | |
| 24 | BTK | |
| 25 | ASC2 | |
| 26 | BUBR1 | |
| 27 | ARTS | |
| 28 | PCAF | |
| 29 | Raf1 | |
| 30 | MSK1 | |
| 31 | SNAP25 | |
| 32 | APRIL | |
| 33 | DAPK | |
| 34 | RAIDD | |
| 35 | HAT1 | |
| 36 | PSF | |
| 37 | HDAC1 | |
| 38 | Rad17 | |
| 39 | Surviving | |
| 40 | SLIPR | |
| 41 | MAG13 | |

TABLE 5
Proteins Downregulated in NCIES0808 cells
treated for 6 Hours with 50 and 100 μM CoQ10.

| | 50 μM CoQ10 6 hr | 100 μM CoQ10 6 hr |
|---|---|---|
| 1 | PRMT3 | α E-Catenin |
| 2 | HDAC2 | Grb2 |
| 3 | Nitric Oxide Synthase bNOS | Bax |
| 4 | Acetyl phospho Histone H3 AL9 | E2F2 |
| | S10 | |
| 5 | MTA 2 | Kaiso |
| 6 | Glutamic Acid Decarboxylase | Glycogen Synthase Kinase 3 |
| | GAD65 67 | |
| 7 | KSR | ATF2 |
| 8 | HDAC4 | HDRP MITR |
| 9 | BOB1 OBF1 | Neurabin I |
| 10 | a1Syntrophin | AP1 |
| 11 | BAP1 | Apaf1 |
| 12 | Importina 57 | |
| 13 | MDC1 | |
| 14 | Laminin2 a2 | |
| 15 | bCatenin | |
| 16 | FXR2 | |
| 17 | AnnexinV | |
| 18 | SMAC Diablo | |
| 19 | MBNL1 | |
| 20 | DImethyl Histone h3 | |
| 21 | Growth factor independence 1 | |
| 22 | U2AF65 | |
| 23 | mTOR | |

### Example 7

### Two-Dimensional Gel Analysis

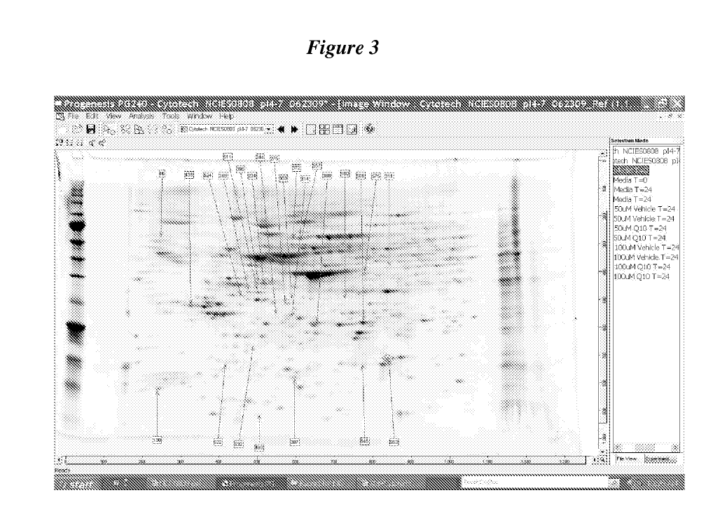

NCIES0808 cells treated for 3, 6 and 24 hours were subjected to 2-D gel electrophoreses and were analyzed to identify protein-level changes relative to the control media samples. A comparative analysis of spots across multiple duplicated gels was performed, comparing the “control media sample” against all of the treated samples at both the 50 uM and 100 uM doses. The analysis included the identification of spot changes over the time course due to an increase, decrease, or post-translational modification. Representative examples of gel images are shown in FIG. 3 and the proteins that are modulated are shown in Table 6.

TABLE 6
Proteins Modulated in NCIES0808 cells
treated for 3, 6, and 24 Hours with 50 and 100 μM CoQ10.

| Protein Identification: | Spot | 50 uM Q10 | 100 uM Q10 |
|---|---|---|---|
| | NCI-0808: 3 hours Q-10: | | |
| | 3 hours | | |
| Proteasome 26S subunit 13; | 240 | −1.3 | 1.5 |
| Endophilin B1 | | | |
| Myosin Regulatory Light Chain | 612 | 1.4 | 1.4 |
| hnRNP C1/C2 | 583 | 1.4 | 1.5 |
| Ubiquilin 1; Phosphatase 2A | 940 | 1 | 1.3 |
| hnRNP C1/C2 | 685 | 1.2 | 1.2 |
| Actin-like 6A; Eukaryotic | 746 | −1.3 | −1.2 |
| Initiation Factor 4All | | | |
| Nuclear Chloride Channel protein | 938 | −1.2 | −1.2 |
| Proteasome 26S subunit | 284 | −1.1 | −1.6 |
| Dismutase Cu/Zn Superoxide | 667 | −1.1 | −1.4 |
| Translin-associated factor X | 154 | 1 | −1.9 |
| | NCI-0808: 6 hours Q-10: | | |
| | 6 hours | | |
| Arsenite translocating ATPase; | 1057 | −1.1 | −1.2 |
| Spermine synthetase | | | |
| ribosomal protein SA | 530 | −1.2 | −1.4 |
| dCTP pyrophosphatase 1 | 720 | −1.2 | −1.2 |
| proteasome beta 3 | 652 | −1.1 | −1.3 |
| proteasome beta 4 | 773 | −1.1 | −1.2 |
| acid phosphatase 1 | 452 | −1.3 | −1.5 |
| diazepam binding inhibitor | 477 | −1.4 | −1.2 |
| | NCI-0808: 24 hours Q-10: | | |
| | 24 hours | | |
| alpha 2-HS glycoprotein | 16 | 1.4 | 5.9 |
| (Bos Taurus, cow) | | | |
| ribosomal proten P2; histone H2A | 130 | −1.3 | −4.2 |
| beta actin | 180 | 1.3 | 3 |
| hnRNP C1/C2 | 234 | −1.2 | −2.6 |
| heat shock protein 70 kD | 244 | 1.1 | 1.7 |
| microtubule associated protein | 275 | 1.1 | −1.8 |
| beta tubulin | 311 | 1.7 | 2.6 |
| proteasome alpha 3 | 314 | 1.1 | −2.2 |
| ATP dependent helicase II | 363 | 1.2 | 2.1 |
| eukaryotic translation elongation | 369 | 1.1 | −2 |
| factor 1 delta | | | |
| lamin B1 | 372 | 1 | −2.1 |
| SMT 3 suppressor of mif two 3 | 387 | 1 | −1.95 |
| homolog 2 | | | |
| heat shock protein 27 kD | 388 | 1.1 | −1.7 |
| hnRNP C1/C2 | 396 | −1.4 | −2 |
| eukaryotc translation elongation | 436 | 1 | −1.9 |
| factor 1 beta 2 | | | |
| Similar to HSPC-300 | 490 | −1.2 | 1.5 |
| heat shock protein 27 kD | 506 | −1.2 | −1.9 |
| eukaryotic translation elongation | 511 | 1 | −1.6 |
| factor 1 delta | | | |
| eukaryotic translation elongation | 524 | 1 | −1.7 |
| factor 1 delta | | | |
| putative c-myc-responsive isoform 1 | 532 | 1 | 1.4 |
| lamin B1 | 557 | 1 | −1.6 |
| ER lipid raft associated 2 | 575 | 1.1 | 1.6 |
| isoform 1; beta actin | | | |
| Dismutase Cu/Zn Superoxide | 583 | 1.1 | −1.3 |
| DNA directed DNA polymerase | 622 | −1.1 | −1.6 |
| epislon 3; (canopy 2 homolog) | | | |
| signal sequence receptor 1 delta | 646 | 1 | 1.5 |

+ = up regulation by Q10
− = down regulation by Q10
Note:
A “1” indicates that there is no change in the amount of the protein.

From the MASCOT analysis top tier spots were identified earlier. In the second stage of analysis the level two spots were analyzed and based on visual inspection and QC were also submitted for MS identification.

Below, in Table 7 is a list of protein IDs for those proteins the amount of which were modulated in NCIES0808 cells treated with CoQ10 after 3 hours which were identified as “level 2” spots.

TABLE 7
Proteins Modulated in NCIES0808 cells
treated for 3 Hours with CoQ10.

| | |
|---|---|
| 545 | Too Low signal (no ID) |
| 522 | Eukaryotic translation initiation factor 3, subunit 3 gamma |
| 673 | Bilverdin reductase A, Transaldolase 1 |
| 504 | Keratin 1,10; Parathymosin |
| 491 | GST omega 1 |
| 348 | chain B Dopamine Quinone Conjugation to Dj-1 |
| 201 | Proteasome Activator Reg (alpha) |
| 270 | No significant signals (no ID) |
| 233 | T-complex protein 1 isoform A |
| 289 | Beta Actin |
| 401 | Chain A Tapasin ERP57; Chaperonin containing TCP1 |
| 429 | Ubiquitin activating enzyme E1 |
| 346 | Ubiquitin activating enzyme E1; Alanyl-tRNA synthetase |
| 254 | Dynactin 1 |
| 323 | Heat shock protein 60 kd |
| 275 | Beta Actin |
| 356 | Spermidine synthase; Beta Actin |
| 385 | Heat Shock protein 70 kd |

A mitochondrial preparation of NICES0808 sample was also analyzed for proteins and below, in Table 8, is the list of proteins the amount of which was modulated following treatment with CoQ10.

TABLE 8
Proteins Modulated in NCIES0808 cells treated with CoQ10.

| | |
|---|---|
| 108 | retinoblastoma binding protein 4 isoform A |
| 1000 | TAR DNA binding protein |
| 37 | eukaryotic translation elongation factor 1 beta 2 |
| 227 | chaperonin containing TCP1, subunit 3 |
| 172 | cytoplasmic dynein IC-2 |

### Example 8

### Western Blot Analysis

NCIES0808 cells treated for 24 hours with 50 or 100 μM Q10 were subjected to Western blot analysis and were analyzed to identify protein-level changes relative to the control media samples.

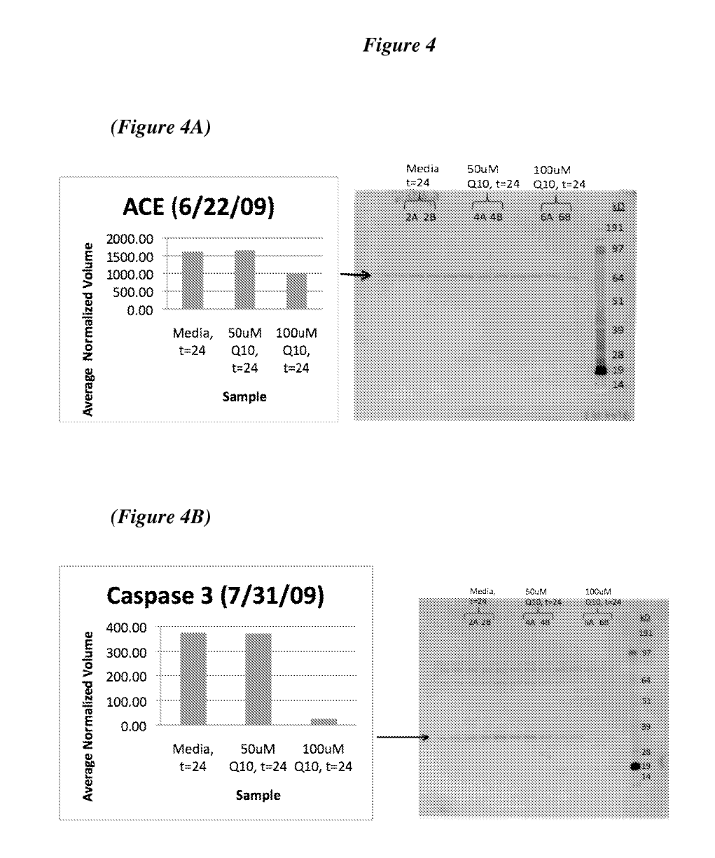

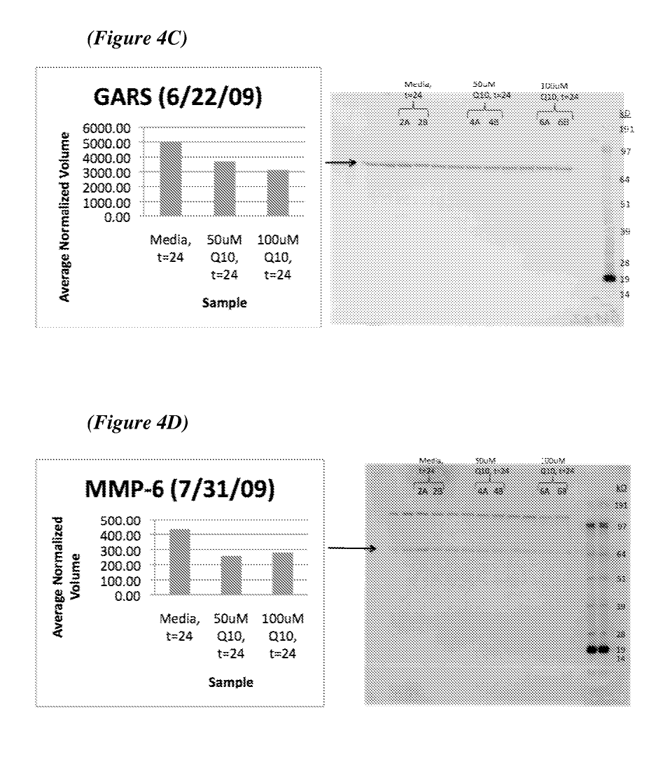

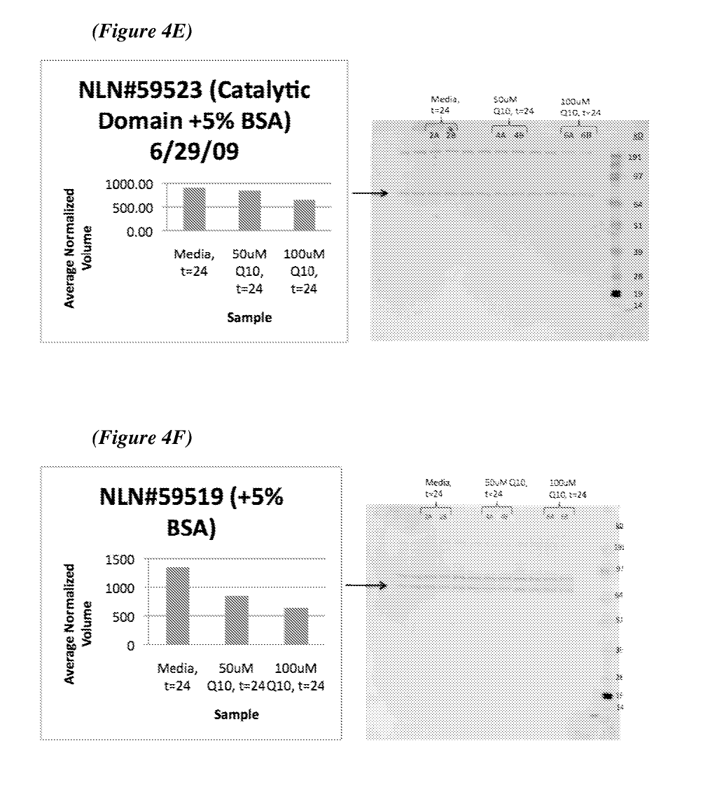

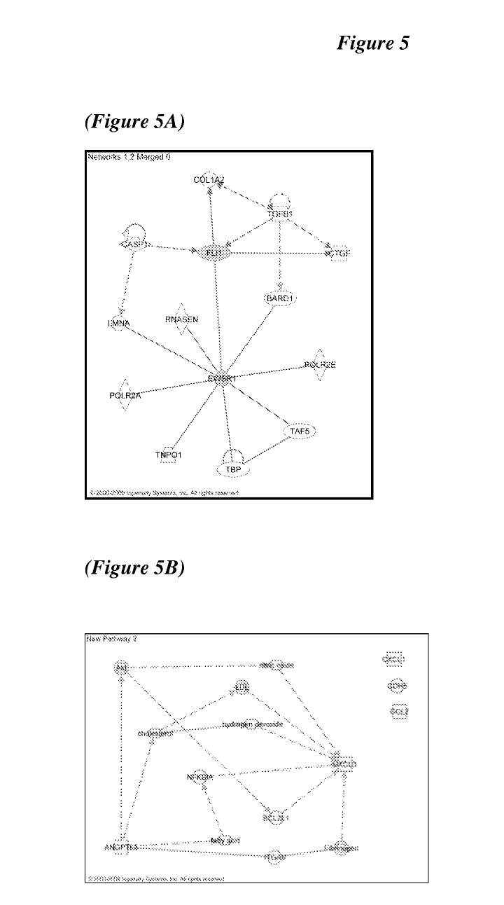

Protein obtained from the treated cells was evaluated by Western blot analysis against an antibody for Angiotensin-converting enzyme (ACE) (FIG. 4A), an antibody for Caspase 3 (FIG. 4B), an antibody for GARS (FIG. 4C), an antibody for Matrix Metalloproteinase 6 (MMP-6) (FIG. 4D) and a series of antibodies for Neurolysin (NLN) (FIGS. 4E-F). The results from these experiments demonstrated that all of the examined proteins were downregulated as a result of cell treatment with Q10. In particular, there was a marked downregulation of Caspase 3 at 24 hours of treatment with 100 μM Q10.

TABLE 9
Proteins modulated in NCIES0808 cells analyzed by Western analysis

| | |
|---|---|
| Angiotensin-converting | downregulated |
| enzyme (ACE) | |
| Caspase 3 | downregulated |
| GARS | downregulated |
| Matrix | downregulated |
| Metalloproteinase 6 | |
| Neurolysin | downregulated |

### Discussion of Examples 4-8

Ewing Sarcoma is a highly aggressive cancer incidence of which does not appear to be associated with Mendelian inheritance, environmental or drug exposure. The most consistent feature of ES is the presence of a fusion gene as a result of chromosomal translocation between the EWSR1 locus and the ETS transcription factor gene. The EWS-ETS fusion genes encode transcription factors such as the EWS-FLI1, aberrant functioning of which is associated with ES pathogenesis. Recent advances in the use of high-throughput (HTS) technologies have begun to provide an understanding of the functional consequence of EWS-FLI1.

The results provided in the Examples above describe the analysis of proteomic data demonstrating the influence of Coenzyme Q10 on key genetic markers that characterize the etiology of Ewing Sarcoma. A combination of antibody array, 2-dimensional gel electrophoresis/mass spectroscopy and real time polymerase chain reaction microarray identified over 90 gene products expression of which appears to be significantly influenced in Ewing Sarcoma cell lines (JDT, 0808) in response to CoQ10 treatment. Of these, expression pattern of approximately 60% of the gene products identified were up-regulated and 40% were down-regulated. Functional groups were identified using “The Database for Annotation, Visualization and Integrated Discovery’ [DAVID] that subdivided the genes in 42 major clusters. Maximum number of genes from the list were segregated within the “Regulation of Cellular Process” and “Metabolic Process” functional groups with the other proteins spread over functional groups including regulation of transcription, programmed cell death, cell development, cytoskeleton, nucleus, proteosome and organ development. Functional assessment of protein and their modulation of cellular events suggest that Ewing cells exposed to CoQ10 induces global expression of cytoskeletal proteins, the resulting destabilization of structural architecture initiates a cellular program culminating in a rapid and robust apoptotic response.

### A. Coenzyme Q10 Modulates Expression of Several Cytoskeletal Proteins: Disruption of Cellular Architecture in the Initiation of Apotosis Response.

Treatment of Ewing Sarcoma cell line with CoQ10 resulted in the altered expression of numerous cytoskeletal components including microfilaments (beta actin, myosin regulatory light chain, actin-related protein ACTL6), intermediate filaments (keratin 1, 10, 13, 17) and microtubules (beta tubulin, microtubule associated protein, dynein), interacting proteins (dynactin) and chaperones (chaperonin containing TCP1). This phenomenon is supported in part by the observed increase in ribosomal proteins (RPLP2), eukaryotic translation initiation factors (EIF3G, EIF4A2) and eukaryotic translation elongation factors (EEF1B2, EEF1D). The corresponding increase in expression of heat shock proteins (HSP27, HSP60, HSP70), and well documented ability of HSP27 to up-regulate expression of actin and stabilize the microtubular structure suggests that CoQ10 mediated alteration in the expression of structural proteins destabilizes the cytoskeletal architecture (Robitaille et al, 2009; Mounier & Arrigo, 2002). The involvement of the cytoskeleton associated changes in the execution of apoptosis e.g. cell rounding, membrane blebbing and chromatin condensation is well established (Mills et al, 1999). However, recent studies suggest that disruption or modulation of the cytoskeleton is a required step in the process of apoptosis (Pawalak & Helfman, 2001). Cytoskeletal disruption by cytochalasin D results in an increase in caspase 3 activation and accelerates DNA-damage induced apoptosis. This effect is recapitulated by the observation that 100 μM CoQ10 caused a 30% increase in Caspase 3 expression within one hour after exposure in Ewing JDT cell line. Given that microtubules such as dyenin (expression of which is increased in response to CoQ10) facilitate transport of p53 to the nucleus in response to DNA damage and tubulin and microtubule associated proteins play an essential role in the process of mitosis, it is suggested that CoQ10 disrupts/destabilizes the cytoskeletal architecture and cell cycle resulting in the activation of programmed cell death.

### B. CoQ10 Dis-Inhibits the EWS-ETS Mediated Repression of Apoptosis Via the CBP/p300 Pathways

One of the proteins up-regulated in response to CoQ10 exposure in the NCIES0808 cell line is the CBP/p300, the CREB-binding protein and its E1A binding protein homologue both of which are well characterized transcriptional co-activators (Chirivia J C et al, 1995; Eckner R et al, 1994). CBP and p300 have similar, interchangeable cellular functions regulating cell growth and development (Janknecht R, 2002; Goodman & Smolik. 2000). CBP/p300 functions as a co-activator for numerous transcriptional factors and appear to serve as bridge/scaffold within the transcriptional machinery (Smolik & Goodman, 2000). There is evidence that the transcriptional activity of EWSR1 gene product in maintenance of normal cellular function is mediated in part via the interaction with the CBP/p300 (Araya et al, 2003; Rossow & Janknecht, 2001). Furthermore, using deletion mutants it was demonstrated that Fli-1 alone and EWS-Fli1 fusion binds to CBP and interferes with the nuclear-receptor transcriptional activity (Ramakrishnan et al, 2004). Evidence of indirect modulation of EWS-ETS fusion proteins by CBP/p300 is based on its ability to interaction with RNA helicase A (RHA), a member of the DEXH family of RNA helicases and RNA polymerase II to modulate transcription (Nakajima T, 1997). Expression of RHA was found in ES cell lines and tumor and a physical interaction between RHA and EWS-FLI1 fusion appears to be enhance the transcriptional and transformational potential of the EWS-FLI1 protein (Toretsky et al, 2006). In fact, it has been proposed that targeting the activity of transcriptional cofactors such as CBP by the EWS-ETS may be responsible in part for the cell transformation (Fujimura et al, 1996). This concept is supported by the observation that the EWS-FLI1 suppressed apoptotic pathways by influencing CBP/p300 pathway (Ramakrishnan et al, 2004). In the same study it was also demonstrated that increasing cellular levels of CBP/p300 sensitized cells to retinoic-acid apoptosis (Ramakrishnan et al, 2004). In the present study, treatment of ES0808 cell line with CoQ10 resulted in an increase in the expression of CBP/p300 (compared to baseline). It is proposed that the CoQ10 mediated increase in CBP/p300 reactivates (i.e. disinhibits) the apoptotic pathways that is usually repressed by EWS-ETS proteins in Ewing Sarcoma.

### C. CoQ10 Induced Cell Death in Ewing Sarcoma Cell Lines is Due to the Activation of the p53 Transcription Factor Regulated Apoptosis.

Multiple lines of evidence support a role for apoptosis in CoQ10 induced cell death in the Ewing Sarcoma model cell lines. The most prominent of these is the involvement of p53 activation demonstrated by a significant increase in its expression in Ewing JDT cell lines one hour after treatment with CoQ10. It is well established that p53 transcription factor is activated in response to cell damage/stress, activating gene expression pathways leading to either cell cycle arrest or apoptosis (Levine, 1997; Giaccia and Kastan, 1998). Furthermore, CBP/p300 interact with p53 and transcriptionally activate p53 dependent MDM2, p21 and Bax promoters (Avantaggiati et al, 1997; Gu et al, 1997; Lill et al, 1997) and acetylate specific lysine residues and augment DNA binding property of p53 (Gu & Roeder, 1997). Thus, CoQ10 directly and/or indirectly increases the expression of p53 in Ewing Sarcoma cell line.

A decrease in Ku70 (also referred to in the art and herein as ATP dependent helicase II) was observed in Ewing Sarcoma ES0808 cell lines treated with CoQ10. Ku70 is associated the proapoptotic protein Bax and has dequbiquitin enzymatic activity (Rathaus et al, 2009). Recent evidence suggest that acetylated p53 has the ability to prevent and disrupt the Ku70-Bax complex to enhance apoptosis (Yamaguchi et al, 2009). Thus, it is suggested that CoQ10 induced decrease in Ku70 in consort with increased p53 activity could augment the pro-apoptotic activity of Bax.

Treatment with CoQ10 resulted in the down-regulation of the heterogenous nuclear ribonucleoprotein C (hnRNP C1/C2) expression that persisted up to 24 hours. The hnRNP C1/C2 proteins are part of the complex that forms the X-linked inhibitor of apoptosis (XIAP) and the internal ribosome entry site (IRES) (Holcik et al, 2003). XIAP is the most powerful intrinsic inhibitor of apoptosis and binds caspase 3, caspase 7 and caspase 9 and inhibit their activities (Deveraux et al, 1997). The over-expression of hnRNP C1/C2 specifically enhanced translation of the XIAP IRES suggesting a role in the modulation of XIAP expression (Holcik et al, 2003). It is proposed that reduction in hnRNP C1/C2 expression decreases XIAP expression and augments the sensitivity of Ewing Sarcoma cell line to CoQ10 induced apoptosis. This hypothesis is supported by the significant increase in Caspase 3 expression observed in Ewing JDT Sarcoma cell line one hour after CoQ10 treatment. The observation that hnRNP C1/C2 co-purified with EWS protein (Zinszner et al, 1994) suggest a novel pathway for the regulation of XIAP and the anti-apoptotic potential of EWS-FLI1 fusions.

Ewing Sarcoma ES0808 cell line treated with CoQ10 demonstrated sustained increases in the expression of various subunits that make-up the proteosome including proteosome subunits PSMA3, PSMB3, PSMB4 and ubiquitin enzymes (ubiquilin). The proteosome is a large multi protein complex that recognizes, bind and degrades proteins marked by a polyubiquitin tag. Since the process of apoptosis is accompanied by progressive decrease in cell size, the proteosomes are essential for degradation of the cytoplasmic and nuclear proteins (Wojcik, 1999). Indeed, activation of the proteosome system during apoptosis has been previously reported (Drexler, 1998; Piedimonte, 1999).

Other proteins the modulation of which supports a role for apoptotic and other pathways, such as destabilization of cell structural architecture, in CoQ10-induced cytotoxicity (e.g., inhibition of tumor cell growth or activation of apoptosis) of Ewing Sarcoma cells include:
(a) Increase in JAB1 expression: JAB 1 (Jun activation binding domain or CSN5) is part of the COP9 signalosome regulating multiple signaling pathways. JAB 1 is a BcLGs-specific binding protein ad enhances the BH3 domain dependent proapoptotic pathways (Liu X, et al. Cell Signaling 20(1): 230-240, 2008.).
(b) Increase in p53R2 expression: Ribonucleoside diphosphate reductase is an enzyme involved in nuclear and mitochondrial DNA synthesis and repair. p53R2 expression is induced by p53 following DNA damage. Over expression of p53R2 interferes with regulation of p53 dependent DNA repair pathway and increases sensitivity of cells to anticancer drugs (Yamaguchi T, et al. Cancer Res. 2001 Nov. 15; 61(22):8256-62.; Nakamura Y: Cancer Sci. 95(1):7-11, 2004.; Pontarin G, et al. Proc Natl Acad Sci USA. 105(46):17801-6, 2008.).
(c) Increase in expression of phosphatidylserine receptor: These receptors are expressed on cell surface of antigen presenting cells (APCs) like macrophages and dendritic cells. These can potentially interact with phosphatidylserine or secreted phoshphatidylserine that emanates from apoptotic cells and promote the anti-inflammatory response by aiding in the recruitment of tumor macrophages (Kim J S, et al. Experimental Molecular Med. 37(6):575-87, 2005.).
(d)
(e) Increase in expression of cytokeratin peptides 13 and 17: Cytokeratin peptides belong to a family of intra-cytoplasmic cytoskeletal proteins, dysregulated expression of which has been implicated in basal cell carcinomas (BCC) (Lo B K, et al. Am J Pathol. 176(5):2435-46, 2010.). Cytokeratins expression is one of the most consistent markers for diagnosis of lung and colorectal adenocarcinomas (Kummar S, et al. Br J Cancer. 86(12):1884-7, 2002.). Although cytokeratin peptides (e.g. 18) is known to be an end-product of caspase 3 proteolysis, not much has been reported about peptides 17 and 13 as products of the apoptosis cascade. CK 17 however has been shown to colocalize with chemokine receptor that have a role in leucocyte chemotaxis in BCC tumorigenesis. It is likely that these products are either the effect of increased apoptosis or the cause of altered tumorigenesis in treated NCI0808 cells.
(f) Increase in expression of neurofilament 160 and 200: Neurofilaments 160 and 200 are respective isoforms of intermediate filament proteins expressed in neuronal cells. Ewing sarcomas are of neuronal origin, abnormal expression of the 200 kD isoform has been observed in a EWS cell line (Lizard-Nacol S, et al. Tumour Biol. 13(1-2):36-43, 1992.).
(g) Increase in expression of Rab5: Rab 5 is a small GTPase involved in autophagy and processing of apoptotic cells in phagosomes (Kinchen J M, et al. Nature. 464(7289):778-82, 2010.). Its increased expression in NCI0808 cells treated with CoQ10 represents the terminal stages of post apoptotic events.
(h) Increase in expression of AFX: Also known as FOXO4, it is a member of the fork head transcription factor family. FOXO4 is regulated by NAD dependent deacetylase SIRT1 and acetyl transferases, CBP/p300. FOXO4 activates oxidative stress response (MnSOD), DNA repair (GADD45), Cell cycle arrest (p27Kip1) and apoptosis (Bim and Fas ligand) genes (Giannakou M E, et al. Trends Cell Biol. 14(8):408-12, 2004). Increased expression of AFX is consistent with increased susceptibility of NCl0808 cells to CoQ10 induced cell death. FOXO1a is also up regulated upon treatment with 100 μM CoQ10,
(i) Increase in expression of MEKK4: Also known as MAP3K4, it is a mitogen activated protein kinase kinase 4, that regulates its downstream mitogen activated kinases, p38 and cJun N terminal kinase (JNK). Activation of MEKK4 in cardiomyocytes has been shown to cause increased levels of apoptosis (Mizote I, et al. J Mol Cell Cardiol. 48(2):302-9, 2010). Increase in MEKK4 expression in NCI0808 cells treated with 50 μM CoQ10 might be representative of ongoing apoptosis in response to the treatment.
(j) Decrease HDAC2 expression: CBP/p300 interacts with HDAC2 to increase promoter activity of Bcl2, the activity is mitigated in the presence of HDAC inhibitors (Duan H, et al. Molecular and Cellular Biology. 25(5): 1608-1619, 2005). Thus, a decrease in HDAC2 expression in response to CoQ10 should decrease promoter activity (and associate antiapoptotic function) of Bcl2.
(k) Decrease HDAC4 expression: CBP/p300 interaction with HDAC4 is involved in the transcriptional regulation of HIF-1α (Seo H-W, et al. FEBS Letters 583:55-60, 2009; Buchwald M, et al. Cancer Letters. 280: 160-167, 2009.). Thus, decrease in the expression of HDAC4 by CoQ10 should decrease the transcriptional activation of HIF-1α and down-stream signaling cascades associated with cellular transformation and oncogenesis.
(l) Increase in PDK1 expression: Phosphoinositide 3 phosphate dependent kinase 1 (PDK1) is the master regulator of AKT and plays a role in cell survival through AKT signaling. Recently constitutive activation of MEK/ERK and PI3K/AKT signaling complexes has been reported in Ewing Sarcoma Family Tumors (ESFT) (Benini S. et al. Int J Cancer. 108(3):358-66, 2004; Liu L Z et al. Cancer Res. 67(13):6325-32, 2007). The elevated expression of these signaling enzymes including PDK1 in response to anti-cancer therapeutics has also been reported (Kawaguchi W, et al.: Cancer Sci. 98(12):2002-2008, 2007, Liu S Q, et al. Dig Liver Dis. 2006 May; 38(5):310-318, 2006) Inhibition of PDK1 and MAPK in combination with anti-cancer drugs in ESFT has been a very successful strategy in development of cancer therapeutics (Yamamoto T, et al. J Cancer Res Clin Oncol. 135(8):1125-36, 2009).
(m) Increase in Caspase12 expression: These belong to the broad family of cysteine proteases that are important mediators of ER stress specific apoptosis. Although ER stress is not known to be an important component in EWS, it is postulated that CoQ10 treatment triggers the ER stress. Previous studies with anti-cancer agents like cisplatin has been shown to lead to an increase of caspase 12 mediated ER stress specific apoptosis (Liu H, et al. J Am Soc Nephrol. 16(7):1985-92, 2005).
(n) Increase in expression of phospholipase D1: This is a phosphatidylcholine specific phospholipase D that is involved in signaling events that regulate mitosis/cell proliferation and membrane trafficking. A study involving over expression and RNAi knockdown of the EWS/FLi or FLi demonstrated that only PLD2 and not PLD1 gene expression was altered (Kikuchi R, et al. Oncogene. 26(12):1802-10, 2007). They also showed that the 5′ promoter in the PLD1 gene lacked the binding sequence for the EWS/FLi fusion proteins. However PLD1 has been shown to be essential for cell survival and protection from apoptosis. Cleavage of PLD1 by caspases promotes apoptosis via modulation of p53 dependent cell death pathways (Jang Y H, et al. Cell Death Differ. 15(11):1782-93, 2008).
(O) Increase in expression of p34 cdc2 kinase & p34 BP1: p34cdc2 is a kinase that regulates the entry of cells into the M phase. Premature activation of p34cdc2 causes cell cycle arrest and initiation of apoptosis. Anti-cancer agents like taxol induces premature activation of p34cdc2 leading to apoptosis in EWS (Duan, H., et al., 2005; Lee S., et al. Cancer Res. 62(20):5703-10, 2002.). An increase in p34cdc2 and binding protein (p34 BP1) expression in response to CoQ10 suggests an increase in CoQ10 induced apoptotic activity in NCl0808 cells.
(p) Increase in expression of Bruton agammaglobulinemia Tyrosine Kinase (BTK): BTK is involved in activation of phospholipase γ2, leading up to intracellular calcium release, extracellular calcium influx and PKC activation. BTK have been reported to directly bind and interact with EWS protein (Bajpai U D, et al. J Exp Med. 191(10):1735-44, 2000), although its exact role in EWS is not known. Since BTKs activate PKC suggests that they mediate the calcium triggered apoptosis in cancer cells (Zhu, D-M., et al. Clin. Cancer Res., 5: 355-360, 1999.).
(q) Increase in expression of ASC2: Apoptosis-associated speck like protein containing a CARD domain (caspase recruitment domain)—belongs to the class of pyrine domain containing proteins and are key components of pathways that regulate inflammation, apoptosis and cytokine processing. These proteins utilize the pyrine domain to activate NFkb and caspase 1 (Stehlik C, et al. Biochem J. 373(Pt 1):101, 2003). It is proposed that these proteins are involved in mediating apoptosis in NCI0808 in response to CoQ10.
(r) Increase in expression of BubR1: BubR1 serves as a mitotic check point serine/threonine protein kinase that is essential for regulating the Anaphase promoting complex (APC/C) (Choi et al 2009). Disruption of this protein leads mitotic arrest and apoptosis of cancer cells (Xu H Z, et al. Cell Cycle. 9(14):2897-907, 2010) Impaired spindle checkpoint has been described in many forms of cancer and an increased expression of BubR1 is likely to be consistent with response to CoQ10.
(s) Increase in expression of PCAF: PCAF is a histone acetyl transferase enzyme that acetylates both histone and non histone proteins. It is involved in mediating a variety of functions including apoptosis.
(t) Increase in expression of Raf1: Raf1 is a proto-oncogene and functions as a serine threonine protein kinase that regulates G2/M exit from the cell cycle. It is involved in the transduction of mitogenic signals from the cell membrane to the nucleus, represents a subset of the Ras-dependent signaling pathway from receptors to the nucleus.
(u) Increase in expression of MSK1: MSK1 is a mitogen and stress activated protein kinase 1 that is directly activated by MAPK and SAPK/p38 and in turn may activate CREB proteins (Deak M, et al. EMBO J. 17(15):4426-41, 1998). Suppression of active CREB induces apoptosis and inhibits cell growth in human non small cell lung cancer.
(v) Increase in expression of SNAP25: The SNAP-25 protein is a component of the SNARE complex, and is involved in assembly of channels in presynaptic neuronal membrane. EWS/Fli chimeric proteins inhibits neuronal differentiation and expression of SNAP25 by regulating Brn-3a, a transcription factor that regulates SNAP25 (Gascoyne D M, et al. Oncogene. 23(21):3830-40, 2004). CoQ10 may inhibit the activities of the EWS/Fli chimeric protein in treated NCIES 0808 cells.
(v) Decrease in expression of mTOR: Mammalian target of rapamycin also known as mechanistic target of rapamycin or FK506 binding protein 12-rapamycin associated protein 1 (FRAP1) is a protein which in humans is encoded by the FRAP1 gene (Brown E J, et al. Nature 369 (6483): 756-8, 1994; Moore P A, et al. Genomics 33 (2): 331-2, 1996). mTOR is a serine/threonine protein kinase that regulates cell growth, cell proliferation, cell motility, cell survival, protein synthesis, transcription and belongs to the phosphatidylinositol 3-kinase-related kinase protein family (Hay N, et al. Genes Dev 18 (16): 1926-45, 2004; Beevers C., et al. Int J Cancer 119 (4): 757-64, 2006). mTOR plays a central role in signaling caused by nutrients and mitogens such as growth factors to regulate translation. mTOR integrates the input from upstream pathways, including insulin, growth factors (such as IGF-1 and IGF-2), and mitogens. mTOR also senses cellular nutrient and energy levels and redox status (Hay N, et al., 2004). Given its primary role in regulating cellular metabolic/bioenergtic status and the observation that dysregulation of mTOR is associated with cancer, the decrease in expression of mTOR in response to CoQ10 in NCIES 0808 cell line is suggestive of its ability to influence cellular metabolic/bioenergetic status in Ewing Sarcoma.

### Example 9

### Method of Preparing a 0.5 Kg Batch of CoQ10 Cream 3% which Includes CoQ10 21% Concentrate and Alkyl Benzoate

A 0.5 kg batch of CoQ10 cream 3.0% composition was prepared by combining the following phases. Phase A included C₁₂₋₁₅ alkyl benzoate at 4.00% w/w, cetyl alcohol NF at 2.00% w/w, glyceryl stearate/PEG-100 stearate at 4.50% w/w and stearyl alcohol NF at 1.5% w/w. The percentages and amounts are listed in the following table.

TABLE 47

| | | | | Amount |
|---|---|---|---|---|
| Phase | Trade Name | CTFA Name | Percent | (kg) |
| A | CAPRYLIC | C₁₂₋₁₅ alkyl benzoate | 4.000 | 0.0200 |
| A | RITA CA | CETYL ALCOHOL | 2.000 | 0.0100 |
| A | RITA SA | STEARYL ALCOHOL | 1.500 | 0.0075 |
| A | RITAPRO 165 | GLYCERYL STEARATE | 4.500 | 0.0225 |
| | | AND PEG-100 STEARATE | | |

Phase B included diethylene glycol monoethyl ether NF at 5.00% w/w, glycerin USP at 2.00% w/w, propylene glycol USP at 1.50% w/w, phenoxyethanol NF at 0.475% w/w, purified water USP at 16.725% w/w and Carbomer Dispersion, 2% at 40% w/w. The percentages and amounts are listed in the corresponding phase table below.

TABLE 48

| | | | | Amount |
|---|---|---|---|---|
| Phase | Trade Name | CTFA Name | Percent | (kg) |
| B | RITA GLYCERIN | GLYCERIN | 2.000 | 0.0100 |
| B | PROPYLENE | PROPYLENE GLYCOL | 1.500 | 0.0075 |
| | GLYCOL | | | |
| B | TRANSCUTOL P | ETHOXYDIGLYCOL | 5.000 | 0.0250 |
| B | PHENOXY- | PHENOXYETHANOL | 0.475 | 0.0024 |
| | ETHANOL | | | |
| B | ACRITAMER 940, | WATER, | 40.000 | 0.2000 |
| | 2% DISPERSION | PHENOXYETHANOL, | | |
| | | PROPYLENE GLYCOL, | | |
| | | CARBOMER 940 | | |
| B | PURIFIED | WATER | 16.725 | 0.0836 |
| | WATER, USP | | | |

Phase C included lactic acid USP at 0.50% w/w, sodium lactate solution USP at 2.00% w/w, triethanolamine NF at 1.30% w/w and purified water USP at 2.50% w/w. The percentages, amounts and further details are listed in the following table.

TABLE 49

| | | | | Amount |
|---|---|---|---|---|
| Phase | Trade Name | CTFA Name | Percent | (kg) |
| C | TEALAN 99% | TRIETHANOLAMINE | 1.300 | 0.0065 |
| C | RITALAC LA | LACTIC ACID | 0.500 | 0.0025 |
| C | RITALAC NAL | SODIUM LACTATE, | 2.000 | 0.0100 |
| | | WATER | | |
| C | PURIFIED | WATER | 2.500 | 0.0125 |
| | WATER, USP | | | |

Phase D included titanium dioxide USP at 1.00% w/w while Phase E included CoQ10 21% concentrate at 15.00% w/w. The percentages, amounts and further details are listed in the following table.

TABLE 50

| | | | | Amount |
|---|---|---|---|---|
| Phases | Trade Name | CTFA Name | Percent | (kg) |
| D | TITANIUM | TITANIUM DIOXIDE | 1.000 | 0.0050 |
| | DIOXIDE, #3328 | | | |
| E | CoQ10 21% | PROPYLENE | 15.000 | 0.0750 |
| | CONCENTRATE | GLYCOL, | | |
| | | POLYSORBATE 80, | | |
| | | WATER, | | |
| | | UBIQUINONE, | | |
| | | LECITHIN, | | |
| | | PHENOXYETHANOL | | |

All weight percentages are relative to the weight of the entire CoQ10 cream 3.0% composition.

The Phase A ingredients were added to a suitable container and heated to between 70 and 80° C. in a water bath. The Phase B ingredients, not including the Carbomer Dispersion, were added to a suitable container and mixed. The Phase C ingredients were also added to a suitable container and then heated to between 70 and 80° C. in a water bath. The CoQ10 21% concentrate of Phase E was placed in a suitable container and melted between 50 and 60° C. using a water bath. The ingredients were mixed as necessary to assure uniformity. The Carbomer Dispersion was then added to a suitable container (Mix Tank) and heated to between 70 and 80° C. while being mixed. While the ingredients were being mixed, the Phase B ingredients were added to the contents of the Mix Tank while maintaining the temperature. The contents were continually mixed and homogenized. The mixer was then turned off, however, homogenization was sustained. While the homogenization continued, the titanium dioxide of Phase D was added to the Mix Tank. The mixer was then turned on and the contents were mixed and further homogenized until completely uniform and fully extended (check color). Homogenization was then stopped and the batch was cooled to between 50 and 60° C. The mixer was then turned off and the melted CoQ10 21% concentrated was added to the Mix Tank. The mixer was subsequently turned on and the contents mixed/recirculated until dispersion was smooth and uniform. The contents of the Mix Tank were then cooled to between 45 and 50° C. The contents were then transferred to a suitable container for storage until unpacking.

### Example 10

### Treatment of Ewing's Sarcoma Tumors In Vivo

Experiments are carried out to evaluate the efficacy of topical Coenzyme Q10 treatment for Ewing's sarcoma tumors in vivo in an animal model. One or more of the following Ewing's sarcoma cell lines are used in these experiments: TC71, TC32, RD-ES, 5838, A4573, EWS-925, NCI-EWS-94, and NCI-EWS-95 (Kontny H U et al., Simultaneous expression of Fas and nonfunctional Fas ligand in Ewings's sarcoma. Cancer Res 1998; 58:5842-9). NCI-EWS-011 and NCI-EWS-021 cell lines were generated at the National Cancer Institute from tumor tissue obtained from recurrent Ewing's sarcomas. Both resected tumors and the generated cell lines are positive for the t(11; 22) EWS/FLI-1 translocation. The rhabdomyosarcoma line RD4A (Kalebic T, et al., Metastatic human rhabdomyosarcoma: molecular, cellular and cytogenetic analysis of a novel cellular model, Invasion Metastasis 1996; 16:83-96) and the neuroblastoma cell lines CHP-212 and KCNR (Thiele C. Neuroblastoma. In: Masters J, Palsson B, editors. Human cell culture. Vol 1. Boston (MA): Kluwer Academic Publishers; 1999. p. 21-53) are used as negative controls. Cell lines are grown in RPMI-1640 medium supplemented with 2 mM L-glutamine and 0.1% or 10% fetal calf serum (Life Technologies, Gaithersburg, Md.).

Tumor cells are cultured to a confluence of 75%, harvested with trypsin/EDTA, and then washed twice with PBS. Two million Ewing's sarcoma cells are injected in 100 μL of PBS into the gastrocnemius of 4- to 8-week-old female SCID/bg mice (Taconic, Germantown, N.Y.). Each mouse generally develops a single palpable tumor evident at 21-28 days after inoculation. At a tumor volume of 100-500 mm³, mice are randomly assigned to receive topical Coenzyme Q10 at various doses as described herein (e.g., 0.01 to about 0.5 milligrams of coenzyme Q10 per square centimeter of skin or the appropriate equivalent for administration to mice) or vehicle alone (5 or 10 mice per group). Topical doses of Coenzyme Q10 are administered to the mice in a single administration or in multiple (e.g., two, three, four, five or more) cycles or rounds of administration. Tumor dimensions are measured every 1 or 2 days with digital calipers to obtain two diameters of the tumor sphere. The lower extremity volume at the site of the tumor is determined by the formula (D×d²/6)×π, where D is the longer diameter and d is the shorter diameter. Lower extremity volumes without tumor are approximately 50 mm³. Tumor dimensions are compared over time in mice topically treated with Coenzyme Q10 and with vehicle alone to evaluate the efficacy of Coenzyme Q10 in inhibiting growth or proliferation of Ewing's sarcoma tumor cells in vivo.

### REFERENCES

Araya N., et al. Journal of Biological Chemistry. 278:5427-5432, 2003.
Avantaggiati M L, et al. Cell 89:1175-1184, 1997.
Bajpai U D, et al. J Exp Med. 191(10):1735-44, 2000.
Beevers C., et al. Int J Cancer 119 (4): 757-64, 2006.
Benini S et al. Int J Cancer. 108(3):358-66, 2004.
Brown E J, et al. Nature 369 (6483): 756-8, 1994.
Buchwald M, et al. Cancer Letters. 280: 160-167, 2009.
Choi E, Choe H, Min J, Choi J Y, Kim J, Lee H: EMBO J. 28(14):2077-89, 2009.
Chrivia J C, et al. Nature 365:855-859, 1995.
Cohen H Y, et al. Mol Cell. 2004 Mar. 12; 13(5):627-38.
Deak M, et al. EMBO J. 17(15):4426-41, 1998.
Deveraux, Q. L. et al. Nature 388: 300-304, 1997.
Duan H, et al. “Molecular and Cellular Biology. 25(5): 1608-1619, 2005.
Drexler HCA. Apoptosis 3:1-7, 1998.
Eckner R, et al. Genes & Development. 8(8): 869-84, 1994.
Fujimura Y, et al. Oncogene 12(1): 159-167, 1996.
Gascoyne D M, et al. Oncogene. 23(21):3830-40, 2004.
Giaccia, A. J., et al. Genes & Development 12, 2973-2983, 1998.
Giannakou M E, et al. Trends Cell Biol. 14(8):408-12, 2004.
Goodman R H et al. Genes and Development. 14:1553-1577, 2000.
Gu W, et al. Nature 387:819-823, 1997.
Hay N, et al. Genes Dev 18 (16): 1926-45, 2004.
Hirano G, et al. Mol Cancer Res. 8(6):864-72, 2010.
Holcík M, et al. Molecular Cell Biology 23(1):280-8, 2003.
Jang Y H, et al.: Cell Death Differ. 15(11):1782-93, 2008.
Janknecht R. “Histology and Histopathology. 17:657-668, 2002.
Kawaguchi W, et al.: Cancer Sci. 98(12):2002-2008, 2007.
Kikuchi R, et al. Oncogene. 26(12):1802-10, 2007.
Kim J S, et al. Experimental Molecular Med. 37(6):575-87, 2005.
Kinchen J M, et al. Nature. 464(7289):778-82, 2010.
Kummar S, et al. Br J Cancer. 86(12):1884-7, 2002.
Lee S., et al. Cancer Res. 62(20):5703-10, 2002.
Levine, A. J. Cell 88, 323-331 1997.
Lill N L et al. Nature 387:823-827, 1997.
Liu H, et al. J Am Soc Nephrol. 16(7):1985-92, 2005.
Liu L Z et al. Cancer Res. 67(13):6325-32, 2007.
Liu S Q, et al. Dig Liver Dis. 2006 May; 38(5):310-318, 2006.
Liu X, et al. Cell Signaling 20(1): 230-240, 2008.
Lizard-Nacol S, et al.: Tumour Biol. 13(1-2):36-43, 1992.
Lo B K, et al. Am J Pathol. 176(5):2435-46, 2010.
Mills J C, et al. Journal of Cell Biology 146:703-707, 1999.
Mizote I, et al. J Mol Cell Cardiol. 48(2):302-9, 2010.
Moore P A, et al. Genomics 33 (2): 331-2, 1996.
Mounier N, et al. Cell Stress and Chaperons 7(2): 167-176, 2002.
Nakajima T, et al. Cell. 90(6): 1107-12, 1997.
Nakamura Y: Cancer Sci. 95(1):7-11, 2004.
Pawalak G, et al. Current Opinions in Genetics & Development. 11:41-47, 2001.
Piedimonte G, et al. Experimental Cell Research 248:381-390, 1999.
Pontarin G, F et al.: Proc Natl Acad Sci USA. 105(46):17801-6, 2008.
Rossow K L et al. I 61(6): 2690-2695, 2001.
Ramakrishnan R, et al. Oncogene. 23(42): 7087-7094, 2004.
Rathaus M, et al. Cell Cycle 8(12):1843-52, 2009
Robitaille H, et al. J Invest Dermatol. 2009 Aug. 13.
Seo H-W, et al. FEBS Letters 583:55-60, 2009.
Stehlik C, et al. Biochem J. 373(Pt 1):101, 2003.
Toretsky J A, et al. Cancer Research. 66(11): 5574-5581, 2006.
Wojcik C. Cellular and Molecular Life Sciences 56:908-917, 1999.
Xu H Z, et al.: Cell Cycle. 9(14):2897-907, 2010.
Yamaguchi H, et al. Journal of Biological Chemistry. 284(17):11171-11183, 2009.
Yamaguchi T, et al. Cancer Res. 2001 Nov. 15; 61(22):8256-62.
Yamamoto T, et al.: J Cancer Res Clin Oncol. 135(8):1125-36, 2009.
Yap D B S, et al. Oncogene 18:7681-7689, 1999.
Ying M Z, et al. Cancer Biol Ther. 2010 Feb. 25; 9(4).
Zhu, D-M., et al. Clin. Cancer Res., 5: 355-360, 1999.
Zinszner, H.; et al. Genes & Development 8(21), 2513-26, 1994

### EQUIVALENTS

Those skilled in the art will recognize, or be able to ascertain using no more than routine experimentation, many equivalents to the specific embodiments and methods described herein. Such equivalents are intended to be encompassed by the scope of the following claims.

1. A method for treating or preventing a sarcoma in a human, comprising:
topically administering a Coenzyme Q10 molecule to the human such that treatment or prevention occurs.

2. The method of claim 1, wherein the topical administration is via a dose selected for providing efficacy in humans for the sarcoma being treated.

3. The method of claim 1, wherein the sarcoma being treated is not a sarcoma typically treated via topical administration with the expectation of systemic delivery of an active agent at therapeutically effective levels.

4. The method of claim 1, wherein the concentration of said Coenzyme Q10 molecule in the tissues of the humans being treated is different than that of a control standard of human tissue representative of a healthy or normal state.

5. The method of claim 1, wherein the form of said Coenzyme Q10 molecule administered to the human is different than the predominant form found in systemic circulation within the human.

6. The method of claim 1, wherein the treatment occurs via an interaction of the Coenzyme Q10 molecule with a gene selected from the group consisting of ANGPTL3, CCL2, CDH5, CXCL1, CXCL3, PRMT3, HDAC2, Nitric Oxide Synthase bNOS, Acetyl phospho Histone H3 AL9 S 10, MTA 2, Glutamic Acid Decarboxylase GAD65 67, KSR, HDAC4, BOB1 OBF1, a1Syntrophin, BAP1, Importina 57, α E-Catenin, Grb2, Bax, Proteasome 26S subunit 13 (Endophilin B1), Actin-like 6A (Eukaryotic Initiation Factor 4A11), Nuclear Chloride Channel protein, Proteasome 26S subunit, Dismutase Cu/Zn Superoxide, Translin-associated factor X, Arsenite translocating ATPase (Spermine synthetase), ribosomal protein SA, dCTP pyrophosphatase 1, proteasome beta 3, proteasome beta 4, acid phosphatase 1, diazepam binding inhibitor, alpha 2-HS glycoprotein (Bos Taurus, cow), ribosomal protein P2 (RPLP2); histone H2A, microtubule associated protein, proteasome alpha 3, eukaryotic translation elongation factor 1 delta, lamin B1, SMT 3 suppressor of mif two 3 homolog 2, heat shock protein 27 kD, hnRNP C1/C2, eukaryotc translation elongation factor 1 beta 2, Similar to HSPC-300, DNA directed DNA polymerase epislon 3; (canopy 2 homolog), LAMA5, PXLDC1, p300 CBP, P53R2, Phosphatidylserine Receptor, Cytokeratin Peptide 17, Cytokeratin peptide 13, Neurofilament 160 200, Rab5, Filensin, P53R2, MDM2, MSH6, Heat Shock Factor 2, AFX, FLIPg d, JAB 1, Myosine, MEKK4, cRaf pSer621, FKHR FOXO1a, MDM2, Fas Ligand, P53R2, Myosin Regulatory Light Chain, hnRNP C1/C2, Ubiquilin 1 (Phosphatase 2A), hnRNP C1/C2, alpha 2-HS glycoprotein (Bos Taurus, cow), beta actin, hnRNP C1/C2, heat shock protein 70 kD, beta tubulin, ATP dependent helicase II, eukaryotc translation elongation factor 1 beta 2, ER lipid raft associated 2 isoform 1 (beta actin), signal sequence receptor 1 delta, Eukaryotic translation initiation factor 3, subunit 3 gamma, Bilverdin reductase A (Transaldolase 1), Keratin 1,10 (Parathymosin), GST omega 1, chain B Dopamine Quinone Conjugation to Dj-1, Proteasome Activator Reg (alpha), T-complex protein 1 isoform A, Chain A Tapasin ERP57 (Chaperonin containing TCP1), Ubiquitin activating enzyme E1; Alanyl-tRNA synthetase, Dynactin 1, Heat shock protein 60 kd, Beta Actin, Spermidine synthase (Beta Actin), Heat Shock protein 70 kd, retinoblastoma binding protein 4 isoform A, TAR DNA binding protein, eukaryotic translation elongation factor 1 beta 2, chaperonin containing TCP1, subunit 3, cytoplasmic dynein IC-2, Angiotensin-converting enzyme (ACE), Caspase 3, GARS, Matrix Metalloproteinase 6 (MMP-6), Neurolysin (NLN)-Catalytic Domain, and Neurolysin (NLN), ADRB, CEACAM1, DUSP4, FOXC2, FOXP3, GCGR, GPD1, HMOX1, IL4R, INPPL1, IRS2, VEGFA, putative c-myc-responsive isoform 1, PDK 1, Caspase 12, Phospholipase D1, P34 cdc2, P53 BP1, BTK, ASC2, BUBR1, ARTS, PCAF, Raf1, MSK1, SNAP25, APRIL, DAPK, RAIDD, HAT1, PSF, HDAC1, Rad17, Surviving, SLIPR, MAG13, Caspase 10, Crk2, Cdc 6, P21 WAF 1 Cip 1, ASPP 1, HDAC 4, Cyclin B1, CD 40, GAD 65, TAP, Par4 (prostate apoptosis response 4), MRP1, MDC1, Laminin2 a2, bCatenin, FXR2, AnnexinV, SMAC Diablo, MBNL1, DImethyl Histone h3, Growth factor independence 1, U2AF65, mTOR, E2F2, Kaiso, Glycogen Synthase Kinase 3, ATF2, HDRP MITR, Neurabin I, AP1, and Apaf1.

7. The method of claim 2, wherein the Coenzyme Q10 molecule is applied in a topical vehicle to a target tissue at a dose in the range of about 0.01 to about 0.5 milligrams of coenzyme Q10 per square centimeter of skin.

8. (canceled)

9. (canceled)

10. The method of claim 1, wherein the sarcoma is a type of sarcoma in Ewing's family of tumors.

11. The method of claim 10, wherein the type of sarcoma in Ewing's family of tumors is Ewing's sarcoma.

12. A method for inhibiting the activity of the EWS-FLI1 fusion protein in a human comprising:
selecting a human subject suffering from a sarcoma, and
administering to said human a therapeutically effective amount of a coenzyme Q10 molecule, thereby inhibiting the activity of the EWS-FLI1 fusion protein.

13. A method for treating or preventing a sarcoma in a human, comprising:
administering a Coenzyme Q10 molecule to a human in need thereof in a dosing regimen such that the permeability of the cell membranes of the human is modulated and treatment or prevention occurs.

14. The method of any one of claims 1, 12 and 13, further comprising:
upregulating the level of expression of one or more genes selected from the group consisting of LAMA5, PXLDC1, p300 CBP, P53R2, Phosphatidylserine Receptor, Cytokeratin Peptide 17, Cytokeratin peptide 13, Neurofilament 160 200, Rab5, Filensin, P53R2, MDM2, MSH6, Heat Shock Factor 2, AFX, FLIPg d, JAB 1, Myosine, MEKK4, cRaf pSer621, FKHR FOXO1a, MDM2, Fas Ligand, P53R2, Proteasome 26S subunit 13 (Endophilin B1), Myosin Regulatory Light Chain, hnRNP C1/C2, Ubiquilin 1 (Phosphatase 2A), hnRNP C1/C2, alpha 2-HS glycoprotein (Bos Taurus, cow), beta actin, hnRNP C1/C2, heat shock protein 70 kD, microtubule associated protein, beta tubulin, proteasome alpha 3, ATP dependent helicase II, eukaryotic translation elongation factor 1 delta, heat shock protein 27 kD, eukaryotc translation elongation factor 1 beta 2, Similar to HSPC-300, ER lipid raft associated 2 isoform 1 (beta actin), Dismutase Cu/Zn Superoxide, and signal sequence receptor 1 delta, ADRB, CEACAM1, DUSP4, FOXC2, FOXP3, GCGR, GPD1, HMOX1, IL4R, INPPL1, IRS2 and VEGFA, putative c-myc-responsive isoform 1, PDK 1, Caspase 12, Phospholipase D1, P34 cdc2, P53 BP1, BTK, ASC2, BUBR1, ARTS, PCAF, Raf1, MSK1, SNAP25, APRIL, DAPK, RAIDD, HAT1, PSF, HDAC1, Rad17, Surviving, SLIPR, MAG13, Caspase 10, Crk2, Cdc 6, P21 WAF 1 Cip 1, ASPP 1, HDAC 4, Cyclin B1, CD 40, GAD 65, TAP, Par4 (prostate apoptosis response 4), and MRP1;
and/or
downregulating the level of expression of one or more genes selected from the group consisting of ANGPTL3, CCL2, CDH5, CXCL1, CXCL3, PRMT3, HDAC2, Nitric Oxide Synthase bNOS, Acetyl phospho Histone H3 AL9 S10, MTA 2, Glutamic Acid Decarboxylase GAD65 67, KSR, HDAC4, BOB1 OBF1, a1Syntrophin, BAP1, Importina 57, α E-Catenin, Grb2, Bax, Proteasome 26S subunit 13 (Endophilin B1), Actin-like 6A (Eukaryotic Initiation Factor 4A11), Nuclear Chloride Channel protein, Proteasome 26S subunit, Dismutase Cu/Zn Superoxide, Translin-associated factor X, Arsenite translocating ATPase (Spermine synthetase), ribosomal protein SA, dCTP pyrophosphatase 1, proteasome beta 3, proteasome beta 4, acid phosphatase 1, diazepam binding inhibitor, ribosomal protein P2 (RPLP2); histone H2A, microtubule associated protein, proteasome alpha 3, eukaryotic translation elongation factor 1 delta, lamin B1, SMT 3 suppressor of mif two 3 homolog 2, heat shock protein 27 kD, hnRNP C1/C2, eukaryotc translation elongation factor 1 beta 2, Similar to HSPC-300, DNA directed DNA polymerase epislon 3 (canopy 2 homolog), Angiotensin-converting enzyme (ACE), Caspase 3, GARS, Matrix Metalloproteinase 6 (MMP-6), Neurolysin (NLN)-Catalytic Domain, Neurolysin (NLN), MDC1, Laminin2 a2, bCatenin, FXR2, AnnexinV, SMAC Diablo, MBNL1, DImethyl Histone h3, Growth factor independence 1, U2AF65, mTOR, E2F2, Kaiso, Glycogen Synthase Kinase 3, ATF2, HDRP MITR, Neurabin I, AP1, and Apaf1.

15. The method of claim 12 or 13, wherein the treatment occurs via an interaction of said Coenzyme Q10 molecule with a gene selected from the group consisting of ANGPTL3, CCL2, CDH5, CXCL1, CXCL3, PRMT3, HDAC2, Nitric Oxide Synthase bNOS, Acetyl phospho Histone H3 AL9 S 10, MTA 2, Glutamic Acid Decarboxylase GAD65 67, KSR, HDAC4, BOB1 OBF1, a1Syntrophin, BAP1, Importina 57, α E-Catenin, Grb2, Bax, Proteasome 26S subunit 13 (Endophilin B1), Actin-like 6A (Eukaryotic Initiation Factor 4A11), Nuclear Chloride Channel protein, Proteasome 26S subunit, Dismutase Cu/Zn Superoxide, Translin-associated factor X, Arsenite translocating ATPase (Spermine synthetase), ribosomal protein SA, dCTP pyrophosphatase 1, proteasome beta 3, proteasome beta 4, acid phosphatase 1, diazepam binding inhibitor, alpha 2-HS glycoprotein (Bos Taurus, cow), ribosomal protein P2 (RPLP2); histone H2A, microtubule associated protein, proteasome alpha 3, eukaryotic translation elongation factor 1 delta, lamin B1, SMT 3 suppressor of mif two 3 homolog 2, heat shock protein 27 kD, hnRNP C1/C2, eukaryotc translation elongation factor 1 beta 2, Similar to HSPC-300, DNA directed DNA polymerase epislon 3; (canopy 2 homolog), LAMA5, PXLDC1, p300 CBP, P53R2, Phosphatidylserine Receptor, Cytokeratin Peptide 17, Cytokeratin peptide 13, Neurofilament 160 200, Rab5, Filensin, P53R2, MDM2, MSH6, Heat Shock Factor 2, AFX, FLIPg d, JAB 1, Myosine, MEKK4, cRaf pSer621, FKHR FOXO1a, MDM2, Fas Ligand, P53R2, Myosin Regulatory Light Chain, hnRNP C1/C2, Ubiquilin 1 (Phosphatase 2A), hnRNP C1/C2, alpha 2-HS glycoprotein (Bos Taurus, cow), beta actin, hnRNP C1/C2, heat shock protein 70 kD, beta tubulin, ATP dependent helicase II, eukaryotc translation elongation factor 1 beta 2, ER lipid raft associated 2 isoform 1 (beta actin), signal sequence receptor 1 delta, Eukaryotic translation initiation factor 3, subunit 3 gamma, Bilverdin reductase A (Transaldolase 1), Keratin 1,10 (Parathymosin), GST omega 1, chain B Dopamine Quinone Conjugation to Dj-1, Proteasome Activator Reg (alpha), T-complex protein 1 isoform A, Chain A Tapasin ERP57 (Chaperonin containing TCP1), Ubiquitin activating enzyme E1; Alanyl-tRNA synthetase, Dynactin 1, Heat shock protein 60 kd, Beta Actin, Spermidine synthase (Beta Actin), Heat Shock protein 70 kd, retinoblastoma binding protein 4 isoform A, TAR DNA binding protein, eukaryotic translation elongation factor 1 beta 2, chaperonin containing TCP1, subunit 3, cytoplasmic dynein IC-2, Angiotensin-converting enzyme (ACE), Caspase 3, GARS, Matrix Metalloproteinase 6 (MMP-6), Neurolysin (NLN)-Catalytic Domain, and Neurolysin (NLN), ADRB, CEACAM1, DUSP4, FOXC2, FOXP3, GCGR, GPD1, HMOX1, IL4R, INPPL1, IRS2, VEGFA, putative c-myc-responsive isoform 1, PDK 1, Caspase 12, Phospholipase D1, P34 cdc2, P53 BP1, BTK, ASC2, BUBR1, ARTS, PCAF, Raf1, MSK1, SNAP25, APRIL, DAPK, RAIDD, HAT1, PSF, HDAC1, Rad17, Surviving, SLIPR, MAG13, Caspase 10, Crk2, Cdc 6, P21 WAF 1 Cip 1, ASPP 1, HDAC 4, Cyclin B1, CD 40, GAD 65, TAP, Par4 (prostate apoptosis response 4), MRP1, MDC1, Laminin2 a2, bCatenin, FXR2, AnnexinV, SMAC Diablo, MBNL1, DImethyl Histone h3, Growth factor independence 1, U2AF65, mTOR, E2F2, Kaiso, Glycogen Synthase Kinase 3, ATF2, HDRP MITR, Neurabin I, AP1, and Apaf1.

16. The method of any one of claims 1, 12 and 13 further comprising a treatment regimen selected from the group consisting of surgery, radiation, hormone therapy, antibody therapy, therapy with growth factors, cytokines, chemotherapy, and allogenic stem cell therapy.

17. A method for assessing the efficacy of a therapy for treating a sarcoma in a subject, the method comprising:
comparing the level of expression of a marker present in a first sample obtained from the subject prior to administering at least a portion of the treatment regimen to the subject, wherein the marker is selected from the group consisting of the markers listed in Tables 2-9; and
the level of expression of the marker present in a second sample obtained from the subject following administration of at least a portion of the treatment regimen,
wherein a modulation in the level of expression of the marker in the second sample as compared to the first sample is an indication that the therapy is efficacious for treating the sarcoma in the subject.

18. A method of assessing whether a subject is afflicted with a sarcoma, the method comprising:
determining the level of expression of a marker present in a biological sample obtained from the subject, wherein the marker is selected from the group consisting of the markers listed in Tables 2-9; and
comparing the level of expression of the marker present in the biological sample obtained from the subject with the level of expression of the marker present in a control sample, wherein a modulation in the level of expression of the marker in the biological sample obtained from the subject relative to the level of expression of the marker in the control sample is an indication that the subject is afflicted with a sarcoma, thereby assessing whether the subject is afflicted with a sarcoma.

19. A method of prognosing whether a subject is predisposed to developing a sarcoma, the method comprising:
determining the level of expression of a marker present in a biological sample obtained from the subject, wherein the marker is selected from the group consisting of the markers listed in Tables 2-9; and
comparing the level of expression of the marker present in the biological sample obtained from the subject with the level of expression of the marker present in a control sample, wherein a modulation in the level of expression of the marker in the biological sample obtained from the subject relative to the level of expression of the marker in the control sample is an indication that the subject is predisposed to developing a sarcoma, thereby prognosing whether the subject is predisposed to developing a sarcoma.

20. A method of prognosing the recurrence of a sarcoma in a subject, the method comprising:
determining the level of expression of a marker present in a biological sample obtained from the subject, wherein the marker is selected from the group consisting of the markers listed in Tables 2-9; and
comparing the level of expression of the marker present in the biological sample obtained from the subject with the level of expression of the marker present in a control sample, wherein a modulation in the level of expression of the marker in the biological sample obtained from the subject relative to the level of expression of the marker in the control sample is an indication of the recurrence of the sarcoma, thereby prognosing the recurrence of a sarcoma in the subject.

21. A method of prognosing the survival of a subject with a sarcoma, the method comprising:
determining the level of expression of a marker present in a biological sample obtained from the subject, wherein the marker is selected from the group consisting of the markers listed in Tables 2-9; and
comparing the level of expression of the marker present in the biological sample obtained from the subject with the level of expression of the marker present in a control sample, wherein a modulation in the level of expression of the marker in the biological sample obtained from the subject relative to the level of expression of the marker in the control sample is an indication of survival of the subject, thereby prognosing survival of the subject with the sarcoma.

22. A method of monitoring the progression of a sarcoma in a subject, the method comprising:
comparing, the level of expression of a marker present in a first sample obtained from the subject prior to administering at least a portion of a treatment regimen to the subject and the level of expression of the marker present in a second sample obtained from the subject following administration of at least a portion of the treatment regimen, wherein the marker is selected from the group consisting of the markers listed in Tables 2-9, thereby monitoring the progression of the sarcoma in the subject.

23. A method of identifying a compound for treating a sarcoma in a subject, the method comprising:
obtaining a biological sample from the subject;
contacting the biological sample with a test compound;
determining the level of expression of one or more markers present in the biological sample obtained from the subject, wherein the marker is selected from the group consisting of the markers listed in Tables 2-9 with a positive fold change and/or with a negative fold change;
comparing the level of expression of the one of more markers in the biological sample with an appropriate control; and
selecting a test compound that decreases the level of expression of the one or more markers with a negative fold change present in the biological sample and/or increases the level of expression of the one or more markers with a positive fold change present in the biological sample, thereby identifying a compound for treating the sarcoma in a subject.

24. The method of claim 17, wherein the sarcoma is a type of sarcoma in Ewing's family of tumors.

25. The method of claim 24, wherein the type of sarcoma in Ewing's family of tumors is Ewing's sarcoma.

26. The method of claim 17, wherein the sample comprises a fluid obtained from the subject.

27. The method of claim 26, wherein the fluid is selected from the group consisting of blood fluids, vomit, saliva, lymph, cystic fluid, urine, fluids collected by bronchial lavage, fluids collected by peritoneal rinsing, and gynecological fluids.

28. The method of claim 27, wherein the sample is a blood sample or a component thereof.

29. The method of claim 17, wherein the sample comprises a tissue or component thereof obtained from the subject.

30. The method of claim 29, wherein the tissue is selected from the group consisting of bone, connective tissue, cartilage, lung, liver, kidney, muscle tissue, heart, pancreas, and skin.

31. The method of claim 17, wherein the subject is a human.

32. The method of claim 17, wherein the level of expression of the marker in the biological sample is determined by assaying a transcribed polynucleotide or a portion thereof in the sample.

33. The method of claim 32, wherein assaying the transcribed polynucleotide comprises amplifying the transcribed polynucleotide.

34. The method of claim 17, wherein the level of expression of the marker in the subject sample is determined by assaying a protein or a portion thereof in the sample.

35. The method of claim 34, wherein the protein is assayed using a reagent which specifically binds with the protein.

36. The method of claim 17, wherein the level of expression of the marker in the sample is determined using a technique selected from the group consisting of polymerase chain reaction (PCR) amplification reaction, reverse-transcriptase PCR analysis, single-strand conformation polymorphism analysis (SSCP), mismatch cleavage detection, heteroduplex analysis, Southern blot analysis, Northern blot analysis, Western blot analysis, in situ hybridization, array analysis, deoxyribonucleic acid sequencing, restriction fragment length polymorphism analysis, and combinations or sub-combinations thereof, of said sample.

37. The method of claim 17, wherein the level of expression of the marker in the sample is determined using a technique selected from the group consisting of immunohistochemistry, immunocytochemistry, flow cytometry, ELISA and mass spectrometry.

38. The method of claim 17, wherein the marker is a marker selected from the group consisting of ANGPTL3, CCL2, CDH5, CXCL1, CXCL3, PRMT3, HDAC2, Nitric Oxide Synthase bNOS, Acetyl phospho Histone H3 AL9 S10, MTA 2, Glutamic Acid Decarboxylase GAD65 67, KSR, HDAC4, BOB1 OBF1, a1Syntrophin, BAP1, Importina 57, α E-Catenin, Grb2, Bax, Proteasome 26S subunit 13 (Endophilin B1), Actin-like 6A (Eukaryotic Initiation Factor 4A11), Nuclear Chloride Channel protein, Proteasome 26S subunit, Dismutase Cu/Zn Superoxide, Translin-associated factor X, Arsenite translocating ATPase (Spermine synthetase), ribosomal protein SA, dCTP pyrophosphatase 1, proteasome beta 3, proteasome beta 4, acid phosphatase 1, diazepam binding inhibitor, alpha 2-HS glycoprotein (Bos Taurus, cow), ribosomal protein P2 (RPLP2); histone H2A, microtubule associated protein, proteasome alpha 3, eukaryotic translation elongation factor 1 delta, lamin B1, SMT 3 suppressor of mif two 3 homolog 2, heat shock protein 27 kD, hnRNP C1/C2, eukaryotc translation elongation factor 1 beta 2, Similar to HSPC-300, DNA directed DNA polymerase epislon 3; (canopy 2 homolog), LAMA5, PXLDC1, p300 CBP, P53R2, Phosphatidylserine Receptor, Cytokeratin Peptide 17, Cytokeratin peptide 13, Neurofilament 160 200, Rab5, Filensin, P53R2, MDM2, MSH6, Heat Shock Factor 2, AFX, FLIPg d, JAB 1, Myosine, MEKK4, cRaf pSer621, FKHR FOXO1a, MDM2, Fas Ligand, P53R2, Myosin Regulatory Light Chain, hnRNP C1/C2, Ubiquilin 1 (Phosphatase 2A), hnRNP C1/C2, alpha 2-HS glycoprotein (Bos Taurus, cow), beta actin, hnRNP C1/C2, heat shock protein 70 kD, beta tubulin, ATP dependent helicase II, eukaryotc translation elongation factor 1 beta 2, ER lipid raft associated 2 isoform 1 (beta actin), signal sequence receptor 1 delta, Eukaryotic translation initiation factor 3, subunit 3 gamma, Bilverdin reductase A (Transaldolase 1), Keratin 1,10 (Parathymosin), GST omega 1, chain B Dopamine Quinone Conjugation to Dj-1, Proteasome Activator Reg (alpha), T-complex protein 1 isoform A, Chain A Tapasin ERP57 (Chaperonin containing TCP1), Ubiquitin activating enzyme E1; Alanyl-tRNA synthetase, Dynactin 1, Heat shock protein 60 kd, Beta Actin, Spermidine synthase (Beta Actin), Heat Shock protein 70 kd, retinoblastoma binding protein 4 isoform A, TAR DNA binding protein, eukaryotic translation elongation factor 1 beta 2, chaperonin containing TCP1, subunit 3, cytoplasmic dynein IC-2, Angiotensin-converting enzyme (ACE), Caspase 3, GARS, Matrix Metalloproteinase 6 (MMP-6), Neurolysin (NLN)-Catalytic Domain, and Neurolysin (NLN), ADRB, CEACAM1, DUSP4, FOXC2, FOXP3, GCGR, GPD1, HMOX1, IL4R, INPPL1, IRS2, VEGFA, putative c-myc-responsive isoform 1, PDK 1, Caspase 12, Phospholipase D1, P34 cdc2, P53 BP1, BTK, ASC2, BUBR1, ARTS, PCAF, Raf1, MSK1, SNAP25, APRIL, DAPK, RAIDD, HAT1, PSF, HDAC1, Rad17, Surviving, SLIPR, MAG13, Caspase 10, Crk2, Cdc 6, P21 WAF 1 Cip 1, ASPP 1, HDAC 4, Cyclin B1, CD 40, GAD 65, TAP, Par4 (prostate apoptosis response 4), MRP1, MDC1, Laminin2 a2, bCatenin, FXR2, AnnexinV, SMAC Diablo, MBNL1, DImethyl Histone h3, Growth factor independence 1, U2AF65, mTOR, E2F2, Kaiso, Glycogen Synthase Kinase 3, ATF2, HDRP MITR, Neurabin I, AP1, and Apaf1.

39. The method of claim 17, wherein the level of expression of a plurality of markers is determined.

40. The method of claim 17, wherein the subject is being treated with a therapy selected from the group consisting of an environmental influencer compound, surgery, radiation, hormone therapy, antibody therapy, therapy with growth factors, cytokines, chemotherapy, allogenic stem cell therapy.

41. The method of claim 40, wherein the environmental influencer compound is a Coenzyme Q10 molecule.

42. A kit for assessing the efficacy of a therapy for treating a sarcoma, the kit comprising reagents for determining the level of expression of at least one marker selected from the group consisting of the markers listed in Tables 2-9 and instructions for use of the kit to assess the efficacy of the therapy for treating the sarcoma.

43. A kit for assessing whether a subject is afflicted with a sarcoma, the kit comprising reagents for determining the level of expression of at least one marker selected from the group consisting of the markers listed in Tables 2-9 and instructions for use of the kit to assess whether the subject is afflicted with the sarcoma.

44. A kit for prognosing whether a subject is predisposed to developing a sarcoma, the kit comprising reagents for determining the level of expression of at least one marker selected from the group consisting of the markers listed in Tables 2-9 and instructions for use of the kit to prognose whether the subject is predisposed to developing the sarcoma.

45. A kit for prognosing the recurrence of a sarcoma in a subject, the kit comprising reagents for assessing the level of expression of at least one marker selected from the group consisting of the markers listed in Tables 2-9 and instructions for use of the kit to prognose the recurrence of the sarcoma.

46. (canceled)

47. A kit for prognosing the survival of a subject with a sarcoma, the kit comprising reagents for determining the level of expression of at least one marker selected from the group consisting of the markers listed in Tables 2-9 and instructions for use of the kit to prognose the survival of the subject with the sarcoma.

48. A kit for monitoring the progression of a sarcoma in a subject, the kit comprising reagents for determining the level of expression of at least one marker selected from the group consisting of the markers listed in Tables 2-9 and instructions for use of the kit to prognose the progression of the sarcoma in a subject.

49. The kit of claim 42, further comprising means for obtaining a biological sample from a subject.

50. The kit of claim 42, further comprising a control sample.

51. The kit of claim 42, wherein the means for determining the level of expression of at least one marker comprises means for assaying a transcribed polynucleotide or a portion thereof in the sample.

52. The kit of claim 42, wherein the means for determining the level of expression of at least one marker comprises means for assaying a protein or a portion thereof in the sample.

53. The kit of claim 42, further comprising an environmental influencer compound.

54. The kit of claim 42, wherein the kit comprises reagents for determining the level of expression of a plurality of markers.
